(19) United States
(12) Patent Application Publication
(10) Pub. No.: US 2020/0263197 A1
(43) Pub. Date: Aug. 20, 2020

(54) TRANSGENIC SELECTION METHODS AND COMPOSITIONS

(71) Applicant: The Jackson Laboratory, Bar Harbor, ME (US)

(72) Inventors: Albert Cheng, Bar Harbor, ME (US); Nathaniel Jillette, Bar Harbor, ME (US); Menghan Du, Bar Harbor, ME (US)

(73) Assignee: The Jackson Laboratory, Bar Harbor, ME (US)

(21) Appl. No.: 16/755,065

(22) PCT Filed: Oct. 11, 2018
(86) PCT No.: PCT/US2018/055412

§ 371 Date: Apr. 9, 2020

Related U.S. Application Data
(60) Provisional application No. 62/624,629, filed on Jan. 31, 2018.
(60) Provisional application No. 62/616,281, filed on Jan. 11, 2018.
(60) Provisional application No. 62/608,478, filed on Dec. 20, 2017.
(60) Provisional application No. 62/571,672, filed on Oct. 12, 2017.

(51) Int. Cl. C12N 15/85; C07K 14/195

(52) U.S. Cl. CPC C12N 15/85; C07K 2319/92; C07K 14/195

(57) ABSTRACT

The present disclosure provides a split intein selectable marker system for the production and selection of transgenic cells.

### RELATED APPLICATIONS

This application claims the benefit under 35 U.S.C. § 119(e) of U.S. provisional application No. 62/616,281, filed Jan. 11, 2018, U.S. provisional application No. 62/608,478, filed Dec. 20, 2017, U.S. provisional application No. 62/624,629, filed Jan. 31, 2018, U.S. provisional application No. 62/571,672, filed Oct. 12, 2017, which is incorporated by reference herein in its entirety.

### SEQUENCE LISTING

This application contains a Sequence Listing in computer readable form (filename: J022770007WO00-SEQ-HJD; 1.50 MB—ASCII text file; created Oct. 3, 2018), which is incorporated herein by reference in its entirety and forms part of the disclosure.

### BACKGROUND

Selectable markers are widely adopted in transgenesis and genome editing for selecting engineered cells with a desired genotype. Antibiotic resistance genes (encoding antibiotic resistance proteins) provide resistance to specific antibiotics so that only cells expressing these resistance genes survive and multiply. Antibiotic resistance genes/antibiotics available for use in eukaryotic cells include hygB/Hygromycin, neo/Geneticin®/G418, pac/Puromycin, Sh bla/Phleomycin D1 (Zeocin™), and bsd/Blasticidin. Fluorescent proteins, such as green fluorescent protein (GFP) provide another means of cell selection, for example, via fluorescent-activated cell sorting (FACS) techniques or fluorescent microscopy.

### SUMMARY

There is a limited number of antibiotic resistance genes/antibiotics available for use in eukaryotic (e.g., mammalian) cells, thus selection schemes for identifying cells containing multiple transgenes are limited. Not only is there a limited number of distinct genes that confer antibiotic resistance in eukaryotic cells, but simultaneous use of as few as three different antibiotic resistance genes can adversely affect the health of transgenic cells. While antibiotic selection can be performed serially, this process is time-consuming. These limitations on selections schemes for identifying transgenic cells are problematic when there is a need to identify cells into which multiple transgenes have been introduced (e.g., to generate a transgenic organism, e.g., animal model, such as a mouse model).

Provided herein are methods, compositions and kits useful for the production and/or identification of, for example, cells and/or organisms harboring two or more transgenes (e.g., double-transgenics, triple-transgenics, etc.). For example, the compositions and kits may be used for the production and/or identification of cells and/or organisms harboring two, three, or four transgenes. This technology is based, at least in part, on a protein splicing mechanism initiated by an intein auto-processing domain, which facilitates the joining (conjugation) specifically in multi-transgenic cells of multiple (e.g., two, three, or four) separate selectable marker protein fragments (double-transgenic cells, triple-transgenic cells, or quadruple-transgenic cells). Joining of the two, three, four, or more separate selectable marker protein fragments in the multi-transgenic cells produces a full-length selectable marker protein that confers, for example, antibiotic resistance (an antibiotic resistance protein) or is capable of fluorescence under an appropriate wavelength of light (fluorescent protein). Cells expressing a full-length antibiotic resistance gene survive in the presence of the corresponding antibiotic and thus are selected as multi-transgenic (e.g., double-transgenic, triple-transgenic, or quadruple-transgenic) cells. Likewise, cells expressing a full-length functioning fluorescent protein fluoresce under the appropriate wavelength of light and thus are selected as multi-transgenic (e.g., double-transgenic, triple-transgenic, or quadruple-transgenic) cells.

Thus, the present disclosure provides, in some embodiments, methods comprising delivering to a composition comprising eukaryotic cells two or more vectors, wherein each vector comprises (i) a nucleotide sequence encoding a selectable marker protein fragment linked to an N-terminal intein protein fragment and/or a C-terminal intein protein fragment and (ii) a nucleotide sequence encoding a molecule of interest, wherein the intein protein fragments, when joined in frame to form full-length function proteins, catalyze joining of the selectable marker protein fragments to produce a full-length selectable marker protein. For example, when two vectors are delivered to a population of cells (e.g., under transfection conditions), some cells will take up the first vector (the vector is introduced in the cells), some cells will take up the second vector, and some cells will take up both vectors. Only those cells that take up both vectors are capable of expressing a full-length functioning selectable marker protein, thus only those cells are selected as double-transgenic cells.

In some embodiments, methods herein comprising delivering to a composition comprising eukaryotic cells (a) a first vector comprising (i) a nucleotide sequence encoding a first selectable marker protein fragment (e.g., antibiotic resistance protein fragment or fluorescent protein fragment) upstream from a nucleotide sequence encoding an N-terminal intein protein fragment and (ii) a nucleotide sequence encoding a first molecule, and (b) a second vector comprising (i) a nucleotide sequence encoding a C-terminal intein protein fragment upstream from a second selectable marker protein fragment (e.g., antibiotic resistance protein fragment or fluorescent protein fragment) and (ii) a nucleotide sequence encoding a second molecule, wherein the N-terminal intein protein fragment and the C-terminal intein protein fragment catalyze joining of the first selectable marker protein fragment to the second selectable marker protein fragment to produce a full-length selectable marker protein. When the two vectors are delivered to a population of cells (e.g., under transfection conditions), some cells will take up the first vector (the vector is introduced in the cells), some cells will take up the second vector, and some cells will take up both vectors. Only those cells that take up both vectors are capable of expressing a full-length functioning selectable marker protein, thus only those cells are selected as double-transgenic cells.

In other embodiments, methods comprise delivering to eukaryotic cells (a) a first vector comprising (i) a nucleotide sequence encoding an N-terminal fragment of a selectable marker protein (e.g., antibiotic resistance protein or fluorescent protein), which is upstream from a nucleotide sequence encoding an N-terminal fragment of a first intein and (ii) a nucleotide sequence encoding a first molecule of interest, (b) a second vector comprising (i) a nucleotide sequence encoding a C-terminal fragment of the first intein, which is upstream from a nucleotide sequence encoding a central fragment of the selectable marker protein, which is upstream from a nucleotide sequence encoding an N-terminal fragment of a second intein and (ii) a nucleotide sequence encoding a second molecule of interest, and (c) a third vector comprising (i) a nucleotide sequence encoding a C-terminal fragment of the second intein, which is upstream from a nucleotide sequence encoding a C-terminal fragment of the selectable marker protein and (ii) a nucleotide sequence encoding a third molecule of interest, wherein the N-terminal fragment and the C-terminal fragment of the first intein catalyze joining of N-terminal fragment of the selectable marker protein to the central fragment of the selectable markerprotein, and the N-terminal fragment and the C-terminal fragment of the second intein catalyze joining of central fragment of the selectable markerprotein to the C-terminal fragment of the selectable markerprotein, to produce a full-length selectable markerprotein. When the three vectors are delivered to a population of cells (e.g., under transfection conditions), some cells will take up the first vector (the vector is introduced in the cells), some cells will take up the second vector, some cells will take up the third vector, some cells will take up two different vectors, and some cells will take up all three vectors. Only those cells that take up all three vectors are capable of expressing a full-length functional selectable marker protein, thus only those cells are selected as triple-transgenic cells.

In still other embodiments, methods comprise delivering to eukaryotic cells (a) a first vector comprising (i) a nucleotide sequence encoding an N-terminal fragment of a selectable marker protein (e.g., antibiotic resistance protein or fluorescent protein), which is upstream from a nucleotide sequence encoding an N-terminal fragment of a first intein and (ii) a nucleotide sequence encoding a first molecule of interest, (b) a second vector comprising (i) a nucleotide sequence encoding a C-terminal fragment of the first intein, which is upstream from a nucleotide sequence encoding a first central fragment of the selectable marker protein, which is upstream from a nucleotide sequence encoding an N-terminal fragment of a second intein and (ii) a nucleotide sequence encoding a second molecule of interest, (c) a third vector comprising (i) a nucleotide sequence encoding a C-terminal fragment of the second intein, which is upstream from a nucleotide sequence encoding a second central fragment of the selectable marker protein, which is upstream from a nucleotide sequence encoding an N-terminal fragment of a third intein and (ii) a nucleotide sequence encoding a third molecule of interest, and (d) a fourth vector comprising (i) a nucleotide sequence encoding a C-terminal fragment of the third intein, which is upstream from a nucleotide sequence encoding a C-terminal fragment of the selectable marker protein and (ii) a nucleotide sequence encoding a third molecule of interest, wherein the N-terminal fragment and the C-terminal fragment of the first intein catalyze joining of N-terminal fragment of the selectable marker protein to the first central fragment of the selectable marker protein, the N-terminal fragment and the C-terminal fragment of the second intein catalyze joining of first central fragment of the selectable marker protein to the second central fragment of the selectable marker protein, the N-terminal fragment and the C-terminal fragment of the third intein catalyze joining of second central fragment of the selectable marker protein to the C-terminal fragment of the selectable marker protein to produce a full-length selectable marker protein. When the four vectors are delivered to a population of cells (e.g., under transfection conditions), some cells will take up the first vector (the vector is introduced in the cells), some cells will take up the second vector, some cells will take up the third vector, some will take up the fourth vector, some cells will take up two different vectors, some cells will take up three different vectors, and some will take up all four vectors. Only those cells that take up all four vectors are capable of expressing a full-length functional selectable marker protein, thus only those cells are selected as quadruple-transgenic cells.

It should be understood that any one embodiment described herein, including those only disclosed in the examples or one section of the specification, is intended to be able to combine with any one or more other embodiments unless explicitly disclaimed.

### BRIEF DESCRIPTION OF THE DRAWINGS

FIGS. 1A-1B. Split selectable marker for antibiotic co-selection of two separate transgenic vectors. (FIG. 1A) The coding sequence of a selectable marker is split into an N-terminal fragment (MarN) and a C-terminal fragment (kerC) and separately cloned upstream of an N-terminal fragment of a split intein (IntN) and downstream of a C-terminal fragment of the split intein (IntC), respectively, on two different vectors each carrying a different transgene. These vectors are delivered to cells yielding sub-populations of cells containing either one of the vectors or both of the vectors. Only in cells with both vectors expressing the two intein-split selectable marker fragments (“markertrons”) undergo protein trans-splicing to reconstitute a full-length selectable marker, allowing specific selection and enrichment of the double transgenic cells. (FIG. 1B) To screen for split points compatible for inteins for an antibiotic resistance gene, we identified potential split points according to the junctional requirement for the type of intein tested, then cloned the corresponding N-terminal and C-terminal fragments to the split intein scaffolds on lentiviral vectors equipped with TagBFP2 or mCherry fluorescent proteins, which serve as our test transgenes to evaluate selection efficiency. These are delivered into cells via lentiviral transduction. The cells were then split into replicate plates, one subjected to antibiotic selection while the other maintained in non-selective media. Following antibiotic selection, the replicate cultures were analyzed by flow cytometry.

FIGS. 2A-2F. Details of the split points of Intein-split resistance (Intres) genes (also referred to as selectable marker genes) and plasmids. (FIG. 2A) Split points for hygromycin resistance protein (SEQ ID NO: 1). Amino acid sequence of hygromycin resistance protein is presented with clouds labeling the split points characterized in this study. Within the label, the top row indicates the plasmid numbers corresponding to Table 1. The bottom row indicates the residue number of the last amino acid in the N-terminal fragment, the species of the intein used, and the residue number of the first amino acid in the C-terminal fragment. “\hat{ }C” indicates an insertion of a Cysteine. (FIG. 2B) Split points for puromycin resistance protein (SEQ ID NO: 2). Amino acid sequence of puromycin resistance protein is presented with clouds labeling the split points characterized in this study. Within the label, the top row indicates the plasmid numbers corresponding to Table 1. The bottom row indicates the residue number of the last amino acid in the N-terminal fragment, the species of the intein used, and the residue number of the first amino acid in the C-terminal fragment. “\hat{ }C” indicates an insertion of a Cysteine. (FIG. 2C) Split points for neomycin resistance protein (SEQ ID NO: 3). Amino acid sequence of neomycin resistance gene is presented with clouds labeling the split points characterized in this study. Within the label, the top row indicates the plasmid numbers corresponding to Table 1. The bottom row indicates the residue number of the last amino acid in the N-terminal fragment, the species of the intein used, and the residue number of the first amino acid in the C-terminal fragment. (FIG. 2D) Split points for blasticidin resistance protein (SEQ ID NO: 4). Amino acid sequence of blasticidin resistance gene is presented with clouds labeling the split points characterized in this study. Within the label, the top row indicates the plasmid numbers corresponding to Table 1. The bottom row indicates the residue number of the last amino acid in the N-terminal fragment, the species of the intein used, and the residue number of the first amino acid in the C-terminal fragment. (FIG. 2E) Split points for green fluorescent protein (SEQ ID NO: 5). (FIG. 2F) Split points for mScarlet fluorescent protein (SEQ ID NO: 6). Amino acid sequence of mScarlet gene is presented with clouds labeling the split points characterized in this study. Within the label, the top row indicates the plasmid numbers corresponding to Table 1. The bottom row indicates the residue number of the last amino acid in the N-terminal fragment, the species of the intein used, and the residue number of the first amino acid in the C-terminal fragment. “\hat{ }C” indicates an insertion of a Cysteine.

FIG. 3. 2-markertron hygromycin (Hygro) intein-split resistance (Intres) genes. Top schematic shows the split points tested for hygromycin resistance gene. The last residue of the N-terminal fragment is indicated on top of the lollipops. Circle lollipops represent split points using NpuDnaE intein while square lollipops represent those using SspDnaB intein. Crossed-out and shaded lollipops indicate split pairs that failed to endow cells with hygromycin resistance. The column plot below shows the percentage of double-transgenic cells (BFP+ mCherry+) in the non-selected (white columns) and the selected cultures (blue columns) analyzed by flow cytometry.

FIG. 4. 2-markertron puromycin (Puro) Intres genes. Top schematic shows split points tested for puromycin resistance genes while bottom column plots show percentage of double transgenic cells in the non-selected (white columns) and the selected cultures (brown columns).

FIG. 5. 2-markertron neomycin (Neo) resistance genes. Top schematic shows split points tested for neomycin resistance genes while bottom column plots show percentage of double transgenic cells in the non-selected (white columns) and the selected cultures (orange columns).

FIG. 6. 2-markertron blasticidin (Blast) Intres genes. Top schematic shows split points tested for blasticidin resistance gene while bottom column plot shows percentages of double transgenic cells in the non-selected (white columns) and the selected (cyan columns) cultures.

FIGS. 7A-7C. Gateway-compatible lentiviral destination vectors with 2-markertron Intres markers. (FIG. 7A) Gateway-compatible lentiviral destination vector kits for each split Intres marker consists of an N-vector and C-vector. N-vector contains viral LTRs, CAGGS promoter, gateway destination cassette AttL, ccdB gene, chloramphenicol resistance gene that allows LR clonase-mediated recombination of Gateway donor vector carrying transgenes, followed by internal ribosomal entry site (IRES) that allows polycistronic expression of the N-markertron. Similarly, C-vector contains the C-markertron and allows recombination of another transgene. (FIG. 7B) TagBFP2 (as transgene 1) and mCherry (as transgene 2) were cloned into the 2-markertron Intres plasmids by Gateway recombination and delivered to cells by lentiviral transduction, followed by antibiotic selection and flow cytometry analysis. Column plot shows the percentage of BFP+mCherry+ double-positive cell in the selected culture from the 2-markertron hygromycin (Hygro, blue column), puromycin (Puro, brown column), and neomycin (Neo, orange column) experiments versus their corresponding non-selective cultures (white columns). (FIG. 7C) NLS-GFP (as transgene 1) that labels nucleus with GFP fluorescence and lifeAct-mScarlet (as transgene 2) that labels F-actin with mScarlet fluorescence were recombined into lentiviral vectors expressing full non-split hygromycin resistance gene or lentiviral vectors with 2-markertron hygromycin Intres genes and used to transduce U2OS cells to make dual-label cells. Representative fluorescence microscopic images show GFP, mScarlet and merged channels of cells after hygromycin selection for two weeks.

FIGS. 8A-8C. Split mScarlet for fluorescence-mediated co-selection of two separate transgenic vectors. (FIG. 8A) 2-markertron mScarlet proteins. Top schematics shows the split points tested for mScarlet. The last residue of the N-terminal fragment is indicated on top of the lollipops. (FIG. 8B) To screen for NpuDnaE intein-compatible split points for mScarlet, we identified potential split points according to the junctional requirement for NpuDnaE intein, then cloned the corresponding N-terminal and C-terminal fragments to the split inteins scaffolds on lentiviral vectors equipped with TagBFP2 or EGFP fluorescent proteins, which serve as our test transgenes to evaluate the selection efficiency. These are delivered into cells via lentiviral transduction. Cells with both lentiviruses contain the necessary protein splicing machinery and mScarlet fragments to reconstitute the full-length mScarlet fluorescent protein, as well as express both TagBFP2 and EGFP transgenes. Cells were subjected to FACS analysis. Boxed schematic shows an example of FACS analysis of the plasmid pair 33+34. P1 population was gated for forward scatter and side scatter for live singlet cells. From those, 17.8% of cells are double positive for TagBFP and EGFP transgenes. When the P1 cells were further gated for mScarlet-positive (mCherry channel), 99.4% of cells are double positive for TagBFP and EGFP. (FIG. 8C) The column plot below shows the percentage of mScarlet-positive cells of each of the indicated split points. The column plot above shows the percentage of TagBFP+ EGFP+ cells among the P1 cells (white columns) and the mScarlet-positive subset of P1 cells (red columns).

FIGS. 9A-9D. Multi-split selectable markers for co-selection of three or more transgenic vectors. (FIG. 9A) A selectable marker is partitioned into three fragments (M₁, M₂ and M₃). The first marker fragment (M₁) is fused upstream of the N-terminal fragment of the first split intein (I_{N1}). The second marker fragment (M₂) is fused downstream of the C-terminal fragment of the first split intein (I_{C1}) and upstream of the N-terminal fragment of the second split intein (I_{N2}). The third marker fragment (M₃) is fused downstream of the C-terminal fragment of the second split intein (I_{C2}). The first split intein catalyzes the joining of M₁ to M₂ while the second split intein catalyzes the joining of M₂ to M₃, effectively reconstituting the full selectable marker. (FIG. 9B) A design of a k-split selectable marker via an “intein chain” mechanism. Similar to the 3-split scenario, the selectable marker is partitioned into k fragments, and are reconstituted through protein trans-splicing mediated by intervening split inteins. (FIG. 9C) Split points identified from 2-split selectable markers were used in combination to produce 3-split selectable markers. The corresponding fragments were cloned into lentiviral vectors to result in the 3-split selectable marker structure and a reporter fluorescent transgene per vector. Cells were then transduced with viruses prepared from these vectors, split into selective or non-selective media. After appropriate selection period, the cultures were analyzed by flow cytometry. (FIG. 9D) 3-markertron hygromycin (Hygro) Intres. Top schematics shows the split points tested for hygromycin resistance gene, with residue numbers of the last amino acid of the N-terminal fragments indicated above circle or square lollipops, representing NpuDnaE and SspDnaB inteins, respectively. Six 3-markertron hygromycin Intreses were tested, each indicated with a numbered line with circle or square indicating the two split points used for each case. Column plot below shows the percentage of triple transgenic (BFP+ GFP+ mCherry+) cells from the non-selective (white columns) and selective (blue columns) cultures for the 3-markertron hygromycin Intres indicated by the numbers below.

FIGS. 10A-10C. Gateway-compatible lentiviral destination vectors with 3-markertron hygromycin Intres genes. (FIG. 10A) Gateway-compatible lentiviral destination vector with viral LTRs, CAGGS promoter, gateway destination cassette AttL, ccdB gene, chloramphenicol resistance gene that allows LR clonase-mediated recombination of Gateway donor vector carrying transgenes, followed by internal ribosomal entry site (IRES) that allows polycistronic expression of the each of the three 3-split hygromycin markertrons. (FIG. 10B) TagBFP2 (as transgene 1) and EGFP (as transgene 2) and mCherry (as transgene 3) were cloned into the 3-split Intres plasmids by Gateway recombination and delivered to cells by lentiviral transduction, followed by antibiotic selection and flow cytometry analysis. (FIG. 10C) Column plot shows the percentage of BFP+GFP+mCherry+ triple-positive cells in the hygromycin selected (blue columns) versus their corresponding non-selective cultures (white columns).

FIG. 11. Four-split Hygro intres. (a) Markertrons of 4-split hygro intres expressed by four different plasmids. Plasmid 115 expresses a markertron created by fusing amino acid 1˜89 of Hygromycin resistance gene [Hygro(1-89)] to NpuDnaE(N) and a leuzine Zipper A motif (LZA). Plasmid 116 expresses a markertron created by fusing, from N- to C-termini, Leucine Zipper B motif (LZB)-NpuDnaGEP(C), Hygro(90-200) and SspDnaB(N). Plasmid 117 expresses a markertron created by fusing, from N- to C-termini, SspDnaB(C), Hygro(201-240), NpuDnaE(N)-LZA. Plasmid 118 expresses a markertron created by fusing LZB-NpuDnaGEP(C) to Hygro(241-341).

FIGS. 12A-12E. Intres markers allow enrichment of biallelic targeted cells from CRISPR/Cas-mediated knock-in experiments. Targeting construct pairs containing homology arms for AAVS1 safe harbor locus were designed to contain full length (FL) non-split or split Intres markers and tested for ability to enrich for biallelic targeted cells via antibiotic selection. (FIG. 12A) Plasmids 107 and 108 contains FL Neomycin (Neo) resistance gene driven by endogenous PPP1R12C promoter at the AAVS1 locus, FL Hygromycin (Hygro) gene and rtTA Dox-responsive transactivator driven by an EF1a promoter, as well as FL Blasticidin (Blast) expressed as well as EGFP (plasmid 107) and mScarlet (plasmid 108) from a dox-inducible TetO promoter. Plasmid 106 contains Cas9 and an sgRNA targeting the AAVS locus. 2A: self-cleaving 2A peptides. Plasmids 106, 107 and 108 were co-transfected into HEK293T cells, split, and passaged in dox-containing hygromycin, blasticidin or non-selective media for two weeks, and analyzed by flow cytometry to assay efficiency of biallelic targeting. (FIG. 12B) Plasmids 109 and 110 contain similar structure as Plasmids 107 and 108, but having split Blast Intres in place of the FL Blast. (FIG. 12C) Plasmids 111 and 112 contain an EF1a-driven FL Blast and TetO-driven FL Hygro, nitroreductase (NTR), fluorescent protein (EGFP or mCherry) separated by 2A peptides. (FIG. 12D) Plasmids 113 and 114 are similar to Plasmids 111 and 112 but with Hygro Intres in place of FL Hygro. (FIG. 12E) Flow cytometry analysis of cells transfected with Plasmid 106 (Cas9+AAVS-sgRNA) and the indicated targeting construct pairs, two weeks after culturing in dox-containing non-selective media (Selection: None), blasticidin selection media (Blast) and hygromycin selection media (Hygro).

### DETAILED DESCRIPTION

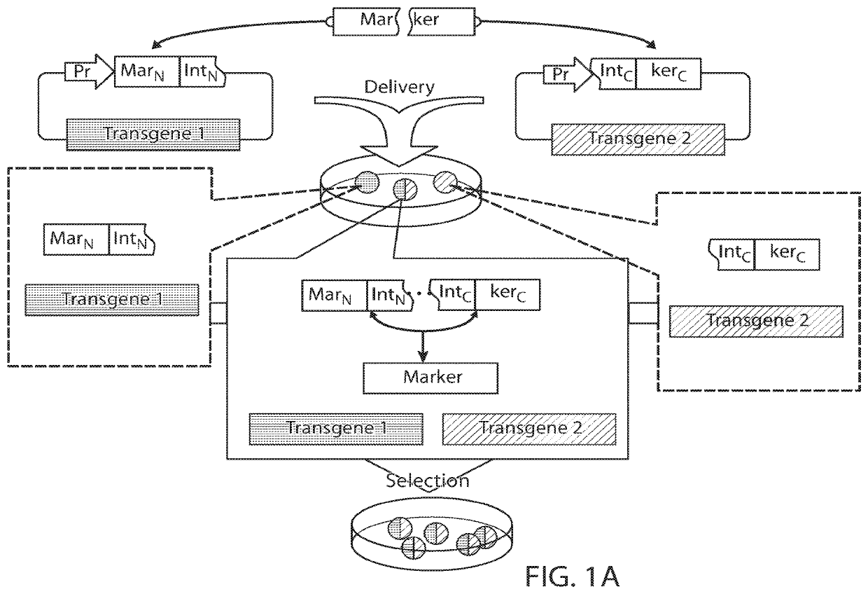

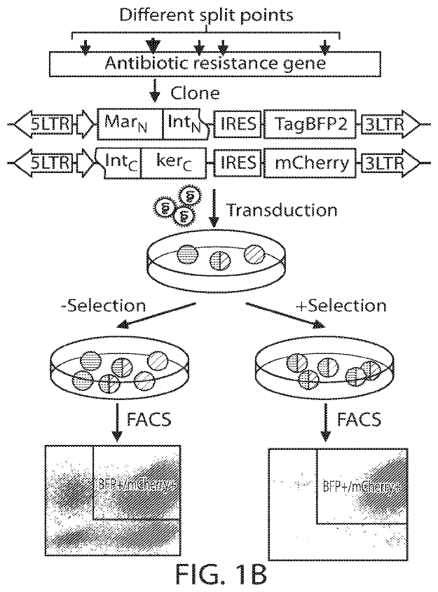

Provided herein, in some aspects, are methods of producing transgenic (e.g., multi-transgenic, such as double transgenic or triple transgenic) organisms, into which more than one transgene (or other genetic element) is introduced. As shown in FIG. 1A, an exemplary method of the present disclosure comprises delivering to a population of cells (a) a vector encoding a first selectable marker protein fragment upstream from an N-terminal intein protein fragment and a first transgene of interest, and (b) another vector encoding a C-terminal intein protein fragment upstream from a second selectable marker protein fragment and a second (e.g., a different) transgene of interest. Some cells of the population will take up a single vector (carrying only a fragment of the intein, a fragment of the selectable marker protein, and a single transgene), while other cells of the population will take up both vectors (and thus both intein fragments, both selectable marker protein fragments, and both transgenes of interest). In cells that take up both vectors, following translation, the intein protein fragments spontaneously and non-covalently assemble (cooperatively fold) into an intein structure to catalyze joining of the first selectable marker protein fragment to the second selectable marker protein fragment to produce a full-length selectable marker protein, which enables specific selection of those double transgenic cells. For example, if the selectable marker protein is an antibiotic resistance protein, only double-transgenic cells expressing the full-length (functional) antibiotic resistance protein will survive selection in the present of the particular antibiotic. If the selectable marker protein is a fluorescent protein, as another example, only double-transgenic cells expressing the full-length (functional) fluorescent protein will emit a detectable signal such that only those signal-emitting cells are selected.

Another exemplary method of the present disclosure comprises delivering to a population of cells (a) a first vector comprising (i) a nucleotide sequence encoding an N-terminal fragment of an antibiotic resistance protein, which is upstream from a nucleotide sequence encoding an N-terminal fragment of a first intein and (ii) a nucleotide sequence encoding a first molecule of interest, (b) a second vector comprising (i) a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein and (ii) a nucleotide sequence encoding a second molecule of interest, and (c) a third vector comprising (i) a nucleotide sequence encoding a C-terminal fragment of the second intein, which is upstream from a nucleotide sequence encoding a C-terminal fragment of the antibiotic resistance protein and (ii) a nucleotide sequence encoding a third molecule of interest. Some cells of the population will take up a single vector (carrying only a fragment of the intein, a fragment of the selectable marker protein, and a single transgene), while other cells of the population will take up two vectors or all three vectors (and thus all intein fragments, all selectable marker protein fragments, and all transgenes of interest). In cells that take up all three vectors, following translation, the intein protein fragments spontaneously and non-covalently assemble (cooperatively fold) into an intein structure to catalyze joining of the N-terminal fragment of the selectable marker protein to the central fragment, and the central fragment to the C-terminal fragment of the selectable marker protein to produce a full-length selectable marker protein, which enables specific selection of those triple-transgenic cells. For example, if the selectable marker protein is an antibiotic resistance protein, only triple-transgenic cells expressing the full-length (functional) antibiotic resistance protein will survive selection in the present of the particular antibiotic. If the selectable marker protein is a fluorescent protein, as another example, only triple-transgenic cells expressing the full-length (functional) fluorescent protein will emit a detectable signal such that only those signal-emitting cells are selected.

### Inteins

An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. In nature, the precursor protein contains three segments—an N-extein (N-terminal portion of the protein) followed by the intein followed by a C-extein (C-terminal portion of the protein). Following splicing, the resulting protein contains the N-extein linked to the C-extein.

There are two types of inteins: cis-splicing inteins are single polypeptides that are embedded in a host protein, whereas trans-splicing inteins (referred to as split inteins) are separate polypeptides that mediate protein splicing after the intein pieces and their protein cargo associate (see, e.g., Paulus, H Annu Rev Biochem 69:447-496 (2000); and Saleh L, Perler F B Chem Rec 6:183-193 (2006)). Split inteins catalyze a series of chemical rearrangements that require the intein to be properly assembled and folded. The first step in splicing involves an N—S acyl shift in which the N-extein polypeptide is transferred to the side chain of the first residue of the intein. This is then followed by a trans-(thio)esterification reaction in which this acyl unit is transferred to the first residue of the C-extein (which is either serine, threonine, or cysteine) to form a branched intermediate. In the penultimate step of the process, this branched intermediate is cleaved from the intein by a transamidation reaction involving the C-terminal asparagine residue of the intein. This then sets up the final step of the process involving an S—N acyl transfer to create a normal peptide bond between the two exteins (Lockless, S W, Muir, T W PNAS 106(27): 10999-11004 (2009)).

To date, there are at least 70 different intein alleles, distinguished not only by the type of host gene in which the inteins are embedded, but also the integration point within that host gene (Perler, F B Nucleic Acids Res. 30: 383-384 (2002); Pietrokovski, S Trends Genet. 17: 465-472 (2001)). A small fraction (less than 5%) of the identified intein genes encode split inteins. Unlike the more common contiguous inteins, split inteins are transcribed and translated as two separate polypeptides, the N-intein and C-intein, each fused to one extein. Upon translation, the intein fragments spontaneously and non-covalently assemble (cooperatively fold) into the canonical intein structure to carry out protein splicing in trans. The first two split inteins to be characterized, from the cyanobacteria Synechocystis species PCC6803 (Ssp) and Nostoc punctiforme PCC73102 (Npu), are orthologs naturally found inserted in the a subunit of DNA Polymerase III (DnaE). Npu is especially notable due its remarkably fast rate of protein trans-splicing (t_{1/2}=50 s at 30° C.). This half-life is significantly shorter than that of Ssp (t_{1/2}=80 min at 30° C.) (Shah, N H et al. J. Am. Chem. Soc. 135: 5839 (2013)).

Herein, split inteins are used to catalyze the joining of two fragments (e.g., an N-terminal fragment and a C-terminal fragment) of a selectable marker protein, such as an antibiotic resistance protein or a fluorescent protein to produce a functional, full-length protein (e.g., FIGS. 1A and 1B).

A split intein may be a natural split intein or an engineered split intein. Natural split inteins naturally occur in a variety of different organisms. The largest known family of split inteins is found within the DnaE genes of at least 20 cyanobacterial species (Caspi J, et al. Mol. Microbiol. 50: 1569-1577 (2003)). Thus, in some embodiments of the present disclosure, a natural split intein is selected from DnaE inteins. Non-limiting examples of DnaE inteins include Synechocystis sp. DnaE (SspDnaE) inteins and Nostoc punctiforme (NpuDnaE) inteins.

In some embodiments, a split intein is an engineered split intein. Engineered split inteins may be produced from contiguous inteins (where a contiguous intein is artificially split) or may be modified natural split inteins that, for example, promote efficient protein purification, ligation, modification and cyclization (e.g., NpuGEP and CfaGEP, as described by Stevens, A J PNAS 114(32): 8538-8543 (2017)). Methods for engineering split inteins are described, for example, by Aranko, A S et al. Protein Eng Des Sel. 27(8): 263-271 (2014), incorporated herein by reference. In some embodiments, the engineered split intein is engineered from DnaB inteins (Wu, H, et al. Biochim Biophys Acta 1387(1-2): 422-432 (1998)). For example, the engineered split intein may be a SspDnaB S1 intein. In some embodiments, the engineered split intein is engineered from GyrB inteins. For example, the engineered split intein may be a SspGyrB S11 intein.

In some embodiments, wherein triple-transgenics are produced, for example, the first intein may be the same as the second intein (e.g., both DnaE inteins). In other embodiments, two different inteins may be used (e.g., a DnaE intein and a DnaB intein). In some embodiments, the first intein is a NpuDnaE intein and the second intein is a NpuDnaE intein.

### Selectable Marker Proteins

Transgenic (e.g., double and/or triple transgenic) cells of the present disclosure are selected based on their expression of a full-length selectable marker protein. A selectable marker protein, generally, confers a trait suitable for artificial selection. Examples of selectable marker proteins include antibiotic resistance proteins and fluorescent proteins.

An antibiotic resistance gene is a gene encoding a protein that confers resistance to a particular antibiotic or class of antibiotics. Non-limiting examples of antibiotic resistance genes for use in eukaryotic cells include those encoding proteins that confer resistance to hygromycin, G418, puromycin, phleomycin D1 or blasticidin. Non-limiting examples of antibiotic resistance genes for use in prokaryotic cells include those encoding proteins that confer resistance to hygromycin, G418, puromycin, phleomycin D1, blasticidin, kanamycin, spectinomycin, streptomycin, ampicillin, carbenicillin, bleomycin, erythromycin, polymyxin D, tetracycline and chloramphenicol.

Hygromycin B is an antibiotic produced by the bacterium Streptomyces hygroscopicus. It is an aminoglycoside that kills bacteria, fungi and higher eukaryotic cells by inhibiting protein synthesis. Hygromycin phosphotransferase (HPT), encoded by the hpt gene (also referred to as the hph or aphIV gene) originally derived from Escherichia coli, detoxifies the aminocyclitol antibiotic hygromycin B. Thus, in some embodiments, the selectable marker gene of the present disclosure is the hpt gene.

G418 (GENETICIN®) is an aminoglycoside antibiotic similar in structure to gentamicin B 1. It is produced by Micromonospora rhodorangea. G418 blocks polypeptide synthesis by inhibiting the elongation step in both prokaryotic and eukaryotic cells. Resistance to G418 is conferred by the neo gene from Tn5 encoding an aminoglycoside 3′-phosphotransferase, APT 3′ II. G418 is an analog of neomycin sulfate, and has similar mechanism as neomycin. Thus, in some embodiments, the selectable marker gene of the present disclosure is the neo gene.

Puromycin is an aminonucleoside antibiotic, derived from Streptomyces alboniger, that causes premature chain termination during translation taking place in the ribosome. Puromycin is selective for either prokaryotes or eukaryotes. Resistance to puromycin is conferred through expression of the puromycin N-acetyl-transferase (pac) gene. Thus, in some embodiments, the selectable marker gene of the present disclosure is the pac gene.

Phleomycin D1 (e.g., ZEOCIN®) is a glycopeptide antibiotic and one of the phleomycins from Streptomyces verticillus belonging to the bleomycin family of antibiotics. It is a broad-spectrum antibiotic that is effective against most bacteria, filamentous fungi, yeast, plant, and animal cells. It causes cell death by intercalating into DNA and induces double strand breaks of the DNA. Resistance to phleomycin D1 is conferred by the product of the Sh ble gene first isolated from Streptoalloteichus hindustanus. Thus, in some embodiments, the selectable marker gene of the present disclosure is the Sh ble gene.

Blasticidin S is an antibiotic that is produced by Streptomyces griseochromogenes. Blasticidin prevents the growth of both eukaryotic and prokaryotic cells by inhibiting termination step of translation and peptide bond formation (to lesser extent) by the ribosome. Resistance to blasticidin is conferred by at least three different genes: bls (an acetyltransferase) from Streptoverticillum spp.; bsr (a blasticidin-S deaminase) from Bacillus cereus (other bsr genes are known as well); and bsd (another deaminase) from Aspergillus terreus. Thus, in some embodiments, the selectable marker gene of the present disclosure is the bls gene, the bsr gene, or the bsd gene.

Non-limiting examples of fluorescent proteins that may be used as provided herein include TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mScarlet, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.

Full-length selectable marker genes, in some embodiments, are produced by joining in the same cell two selectable marker gene fragments. In some embodiments, with reference to any full-length protein, one of the fragments is an N-terminal fragment (N-extein), while the other fragment is a C-terminal fragment (C-extein). Thus, in some embodiments, a first antibiotic resistance protein fragment is an N-terminal antibiotic resistance protein fragment, and a second antibiotic resistance protein fragment is a C-terminal antibiotic resistance protein fragment. In other embodiments, a first fluorescent protein fragment is an N-terminal fluorescent protein fragment, and a second fluorescent protein fragment is a C-terminal fluorescent protein fragment.

In other embodiments, full-length selectable marker genes are produced by joining in the same cell three or more selectable marker gene fragments. In some embodiments, with reference to any full-length protein, one of the fragments is an N-terminal fragment, one or more (e.g., 1, 2, or 3) of the fragments is a central fragment, and one of the fragments is a C-terminal fragment.

An N-terminal fragment may be any protein fragment that includes the free amine group (—NH2) of the full-length protein. A C-terminal fragment may be any protein fragment that includes the free carboxyl group (—COOH). A central fragment may be any protein fragment that is located between the N-terminal fragment and the C-terminal fragment of the full-length protein.

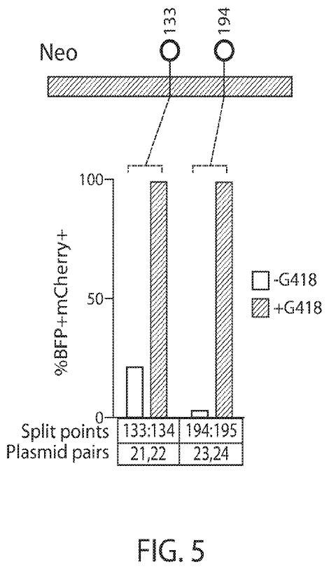

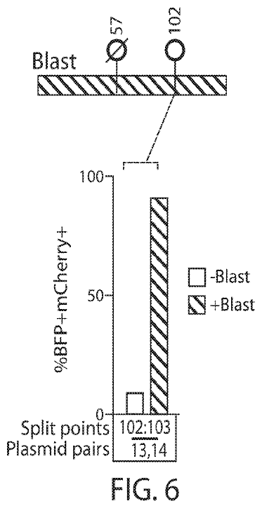

For example, amino acids 1-89 of the gene encoding hygromycin (a 341-amino acid protein) may be referred to as the N-terminal protein fragment, while amino acids 90-341 may be referred to as the C-terminal fragment. Similarly, with reference to FIG. 5, amino acids 1-200 of the gene encoding hygromycin may be referred to as the N-terminal protein fragment, while amino acids 201-341 may be referred to as the C-terminal fragment. FIG. 6 shows additional examples where amino acids 1-53, 1-240, or 1-292 are considered the N-terminal protein fragments of full length hygromycin containing amino acids 54-341, 241-341, or 293-341 as the respective C-terminal fragments.

As another example, amino acids 1-52 of the gene encoding hygromycin (a 341-amino acid protein) may be referred to as the N-terminal protein fragment, amino acids 53-89 may be referred to as the central protein fragment, and amino acids 90-341 may be referred to as the C-terminal fragment. Similarly, amino acids 1-89 of the gene encoding hygromycin may be referred to as the N-terminal protein fragment, amino acids 90-240 may be referred to as the central fragment, and amino acids 241-341 may be referred to as the C-terminal fragment.

### Transgenes and Other Molecules of Interest

The methods and compositions of the present disclosure are used, in some embodiments, to produce multi-transgenic (e.g., double and/or triple transgenic) cells and/or organisms. Thus, in some embodiments, the methods use one vector that encodes a first molecule (a first molecule of interest) and another vector that encodes a second molecule (a second molecule of interest). In some embodiments, the methods use yet another vector that encodes a third molecules of interest. Additional vectors (e.g., encoding additional central fragments of a selectable marker protein) may encode additional molecules of interest. Molecules of interest may be, for example, polypeptides (e.g., proteins and peptides) or polynucleotides (e.g., nucleic acids, such as DNA or RNA).

In some embodiments, the first molecule (e.g., located on the first vector) is a protein. In some embodiments, the second molecule (e.g., located on the second vector) is a protein. In some embodiments, the third molecule (e.g., located on the third vector) is a protein. Examples of proteins of interest include, but are not limited to, enzymes, cytokines, transcription factors, hormones, growth factors, blood factors, antigens and antibodies.

In some embodiments, the first molecule is a peptide. In some embodiments, the second molecule is a peptide. In some embodiments, the third molecule is a peptide.

In some embodiments, the first molecule is a messenger RNA (mRNA). In some embodiments, the second molecule is a mRNA. In some embodiments, the third molecule is a mRNA. The mRNA, in some embodiments, encodes a vaccine or other antigenic molecule.

In some embodiments, the first molecule is a non-coding RNA (a RNA that does not encode a protein). In some embodiments, the second molecule is a non-coding RNA. In some embodiments, the third molecule is a non-coding RNA. Examples of non-coding RNA include, but are not limited to, RNA interference molecules, such as microRNA (miRNA), antisense RNA, short-interfering RNA (siRNA) or short-hairpin RNA (shRNA).

### Vectors

Methods of the present disclosure include the use of at least two or at least three different vectors. A vector is any nucleic acid that may be used as a vehicle to carry exogenous (foreign) genetic material into a cell. A vector, in some embodiments, is a DNA sequence that includes an insert (e.g., transgene) and a larger sequence that serves as the backbone of the vector. Non-limiting examples of vectors include plasmids, viruses/viral vectors, cosmids, and artificial chromosomes, any of which may be used as provided herein. In some embodiments, the vector is a viral vector, such as a viral particle. In some embodiments, the vector is an RNA-based vector, such as a self-replicating RNA vector. In some embodiments, the first vector is a plasmid, the second vector is a plasmid, and/or the third vector is a plasmid. A vector, as provided herein, includes a promoter operably linked to a nucleic acid encoding a fragment of an intein and a fragment of selectable marker protein. In some embodiments, a vector also comprises a promoter operably linked to a nucleic acid, such as a transgene, encoding a molecule of interest.

In some embodiments, one vector (e.g., a first vector) comprises a nucleotide sequence encoding a first selectable marker protein fragment upstream from a nucleotide sequence encoding an N-terminal intein protein fragment, while the other vector (e.g., a second vector) comprises a nucleotide sequence encoding a C-terminal intein protein fragment upstream from a second antibiotic resistance protein fragment (see, e.g., FIG. 1A). This configuration is equivalent to one vector (e.g., a first vector) comprising a nucleotide sequence encoding an N-terminal intein protein fragment downstream from a nucleotide sequence encoding a first selectable marker protein fragment, and the other vector (e.g., a second vector) comprising a second antibiotic resistance protein fragment downstream from a nucleotide sequence encoding a C-terminal intein protein fragment. The terms “upstream” and “downstream” refer to relative positions in a nucleic acid. Each nucleic acid has a 5′ end and a 3′ end, so named for the carbon position on the deoxyribose (or ribose) ring. When considering double-stranded DNA, for example, upstream is toward the 5′ end of the coding strand and downstream is toward the 3′ end.

In some embodiments, (a) a first vector comprises a nucleotide sequence encoding an N-terminal fragment of an antibiotic resistance protein, which is upstream from a nucleotide sequence encoding an N-terminal fragment of a first intein, (b) a second vector comprises a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein, and (c) a third vector comprises a nucleotide sequence encoding a C-terminal fragment of the second intein, which is upstream from a nucleotide sequence encoding a C-terminal fragment of the antibiotic resistance protein. This configuration is equivalent to a (a) a first vector comprising a nucleotide sequence encoding an N-terminal fragment of a first intein, which is downstream from a nucleotide sequence encoding an N-terminal fragment of an antibiotic resistance protein, (b) a second vector comprising a nucleotide sequence encoding an N-terminal fragment of a second intein, which is downstream from a nucleotide sequence encoding a central fragment of the antibiotic resistance protein, which is downstream from a nucleotide sequence encoding a C-terminal fragment of the first intein, and (c) a third vector comprising a C-terminal fragment of the antibiotic resistance protein, which is downstream from a nucleotide sequence encoding a C-terminal fragment of the second intein.

### Cells

Methods of the present disclosure may be used for the production of transgenic cells and organisms by introducing into host cells the vectors (e.g., first and second vectors) described herein. The cells into which the vectors are introduced may be eukaryotic or prokaryotic. In some embodiments, the cells are eukaryotic. Examples of eukaryotic cells for use as provided herein include mammalian cells, plant cells (e.g., crop cells), inset cells (e.g., Drosophila) and fungal cells (e.g., Saccharomyces). Mammalian cells may be, for example, human cells (stem cells or cells from an established cell line), primate cells, equine cells, bovine cells, porcine cells, canine cells, feline cells, or rodent cells (e.g., mouse or rat). Examples of mammalian cells for use as provided herein include, but are not limited to, Chinese hamster ovary (CHO) cells, human embryonic kidney (HEK) 293 cells, HeLa cells, and NSO cells. In some embodiments, the cells are prokaryotic. Examples of prokaryotic cells for use as provided herein include bacterial cells. Bacterial cells may be, for example, Escherichia spp. (e.g., Escherichia coli), Streptococcus spp. (e.g., Streptococcus pyogenes, Streptococcus viridans, Streptococcus pneumoniae), Neisseria spp. (e.g., Neisseria gibirrhoea, Neisseria meningitidis), Corynebacterium spp. (e.g., Corynebacterium diphtheriae), Bacillis spp. (e.g., Bacillis anthracis, Bacillis subtilis), Lactobacillus spp., Clostridium spp. (e.g., Clostridium tetani, Clostridium perfringens, Clostridium novyii), Mycobacterium spp. (e.g., Mycobacterium tuberculosis), Shigella spp. (e.g., Shigella flexneri, Shigella dysenteriae), Salmonella spp. (e.g., Salmonella typhi, Salmonella enteritidis), Klebsiella spp. (e.g., Klebsiella pneumoniae), Yersinia spp. (e.g., Yersinia pestis), Serratia spp. (e.g., Serratia marcescens), Pseudomonas spp. (e.g., Pseudomonas aeruginosa, Pseudomonas mallei), Eikenella spp. (e.g., Eikenella corrodens), Haemophilus spp. (e.g., Haemophilus influenza, Haemophilus ducreyi, Haemophilus aegyptius), Vibrio spp. (e.g., Vibrio cholera, Vibrio natriegens), Legionella spp. (e.g., Legionella micdadei, Legionella bozemani), Brucella spp. (e.g., Brucella abortus), Mycoplasma spp. (e.g., Mycoplasma pneumoniae) or Streptomyces spp. (e.g. Streptomyces coelicolor, Streptomyces lividans, Streptomyces albus).

### Delivery and Selection Methods

Methods of the present disclosure, in some embodiments, include delivering vectors to a composition comprising cells and maintaining the composition under conditions that permit introduction of nucleic acid (e.g., first, second, and third vector) into the cells and permit nucleic acid expression in the cells to produce eukaryotic cells. Conditions required for the introduction of nucleic acid (e.g., vectors) into cells are well known. These conditions include, for example, transformation (of prokaryotic cells) conditions, transfection (of eukaryotic cells) conditions, transduction (via virus/viral vector) conditions, and electroporation conditions, any of which may be used as provided herein. Thus, in some embodiments, methods of the present disclosure include transfecting eukaryotic (e.g. mammalian) cells, while in other embodiments, the methods include transforming prokaryotic (e.g., bacterial) cells.

The selection of transgenic, e.g., multi-transgenic cells, such as double, triple, and/or quarduple transgenic cells depends on the type of selectable marker used. For example, if the selectable marker protein is an antibiotic resistance protein, the selection step may include exposing the cells to a specific antibiotic and selecting only those cells that survive. If the selectable marker protein is a fluorescent protein, the selection step may include simply viewing the cells under a microscope and selecting cells that fluoresce, or the selection step may include other fluorescent selection methods, such as fluorescence-activated cell sorting (FACS) sorting.

In some embodiments, cells are transduced with viral vectors (e.g., viruses) carrying the nucleic acids as described herein. In some embodiments, prior to transduction (or other transfection method), cells are seeded, for example, on well plates (e.g., 12-well plates) at a density of 1×10⁴ to 1×10⁶ per well. In some embodiments 100 μL to 500 μL, e.g., 100, 150, 200, 250, 300, 350, 400, 450, or 500 μL of each viral vector is added to each well.

### Kits

The present disclosure also provides kits that may be used, for example, to produce and screen for transgenic cells and/or organisms. The kits may include any two or more components as described herein. For example, a kit may comprise (a) a first vector comprising a nucleotide sequence encoding a first selectable marker protein fragment upstream from a nucleotide sequence encoding an N-terminal intein protein fragment; and (b) a second vector comprising a nucleotide sequence encoding a C-terminal intein protein fragment upstream from a second selectable marker protein fragment, wherein the N-terminal intein protein fragment and the C-terminal intein protein fragment catalyze joining of the first selectable marker protein fragment to the second selectable marker protein fragment to produce a full-length antibiotic resistance protein.

In some embodiments, the kits include any two or more components as described herein. For example, a kit may comprise (a) a first vector comprising a nucleotide sequence encoding an N-terminal fragment of an antibiotic resistance protein, which is upstream from a nucleotide sequence encoding an N-terminal fragment of a first intein, (b) a second vector comprising a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein, and (c) a third vector comprising a nucleotide sequence encoding a C-terminal fragment of the second intein, which is upstream from a nucleotide sequence encoding a C-terminal fragment of the antibiotic resistance protein, wherein the N-terminal fragment and the C-terminal fragment of the first intein catalyze joining of N-terminal fragment of the antibiotic resistance protein to the central fragment of the antibiotic resistance protein, and the N-terminal fragment and the C-terminal fragment of the second intein catalyze joining of central fragment of the antibiotic resistance protein to the C-terminal fragment of the antibiotic resistance protein, to produce a full-length antibiotic resistance protein.

In some embodiments, the kits further comprise any one or more of the following components: buffers, salts, cloning enzymes (e.g., LR clonase), competent cells (e.g., competent bacterial cells), transfection reagents, antibiotics, and/or instructions for performing the methods described herein.

### ADDITIONAL EMBODIMENTS

Additional embodiments of the present disclosure are encompassed by the following numbered paragraphs:

1. A method comprising delivering to eukaryotic cells

(a) a first vector comprising (i) a nucleotide sequence encoding an N-terminal fragment of an antibiotic resistance protein, which is upstream from a nucleotide sequence encoding an N-terminal fragment of an intein and (ii) a nucleotide sequence encoding a first molecule of interest; and

(b) a second vector comprising (i) a nucleotide sequence encoding a C-terminal fragment of the intein, which is upstream from a C-terminal fragment of the antibiotic resistance protein and (ii) a nucleotide sequence encoding a second molecule of interest,

wherein the N-terminal fragment and the C-terminal fragment of the intein catalyze joining of the N-terminal fragment and the C-terminal fragment of the antibiotic resistance protein to produce a full-length antibiotic resistance protein.

2. The method of paragraph 1 further comprising maintaining the eukaryotic cells under conditions that permit introduction of the first and second vectors into the eukaryotic cells to produce transgenic eukaryotic cells.
3. The method of paragraph 2 further comprising selecting the transgenic eukaryotic cells that comprise the full-length antibiotic resistance protein.
4. The method of any one of paragraphs 1-3, wherein the eukaryotic cells are mammalian cells.
5. The method of any one of paragraphs 1-4, wherein the antibiotic resistance protein confers resistance to hygromycin, G418, puromycin, phleomycin D1 or blasticidin.
6. The method of any one of paragraphs 1-5, wherein the intein is a split intein.
7. The method of paragraph 6, wherein the split intein is a natural split intein.
8. The method of paragraph 7, wherein the natural split intein is selected from DnaE inteins.
9. The method of paragraph 8, wherein the DnaE inteins are selected from Synechocystis sp. DnaE (SspDnaE) inteins and Nostoc punctiforme (NpuDnaE) inteins.
10. The method of paragraph 6, wherein the split intein is an engineered split intein.
11. The method of paragraph 10, wherein the engineered split intein is engineered from DnaB inteins.
12. The method of paragraph 11, wherein the engineered split intein is a SspDnaB S1 intein.
13. The method of paragraph 12, wherein the engineered split intein is engineered from GyrB inteins.
14. The method of paragraph 13, wherein the engineered split intein is a SspGyrB S11 intein.
15. The method of any one of paragraphs 1-14, wherein the first and/or second molecule is a protein.
16. The method of any one of paragraphs 1-15, wherein the first and/or second molecule is a non-coding ribonucleic acid (RNA).
17. The method of paragraph 16, wherein the non-coding RNA is a microRNA (miRNA), antisense RNA, short-interfering RNA (siRNA) or short-hairpin RNA (shRNA).
18. The method of any one of paragraphs 1-17, wherein the first and/or second vector is a plasmid vector or a viral vector.
19. A method comprising delivering to eukaryotic cells

(a) a first vector comprising (i) an N-terminal fragment of a hygB gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the hygB gene and (ii) a second molecule of interest,

wherein the N-terminal fragment and C-terminal fragment of the intein catalyze joining of a protein fragment encoded by the N-terminal fragment of the hygB gene to a protein fragment encoded by the C-terminal fragment of the hygB gene to produce full-length hygromycin B phosphotransferase.

20. The method of paragraph 19, wherein the first amino acid of the protein fragment encoded by the second hygB gene fragment is cysteine.
21. The method of paragraph 23, wherein

the protein fragment encoded by the N-terminal fragment of the hygB gene comprises an amino acid sequence identified by amino acids 1-89 of SEQ ID NO: 1, and the protein fragment encoded by the C-terminal fragment of the hygB gene comprises an amino acid sequence identified by amino acids 90-341 of SEQ ID NO: 1;

the protein fragment encoded by the N-terminal fragment of the hygB gene comprises an amino acid sequence identified by amino acids 1-200 of SEQ ID NO: 1, and the protein fragment encoded by the C-terminal fragment of the hygB gene comprises an amino acid sequence identified by amino acids 201-341 of SEQ ID NO: 1;

the protein fragment encoded by the N-terminal fragment of the hygB gene comprises an amino acid sequence identified by amino acids 1-53 of SEQ ID NO: 1, and the protein fragment encoded by the C-terminal fragment of the hygB gene comprises an amino acid sequence identified by amino acids 54-341 of SEQ ID NO: 1;

the protein fragment encoded by the N-terminal fragment of the hygB gene comprises an amino acid sequence identified by amino acids 1-240 of SEQ ID NO: 1, and the protein fragment encoded by the C-terminal fragment of the hygB gene comprises an amino acid sequence identified by amino acids 241-341 of SEQ ID NO: 1;

the protein fragment encoded by the N-terminal fragment of the hygB gene comprises an amino acid sequence identified by amino acids 1-292 of SEQ ID NO: 1, and the protein fragment encoded by the C-terminal fragment of the hygB gene comprises an amino acid sequence identified by amino acids 293-341 of SEQ ID NO: 1.

22. The method of any one of paragraphs 23-21, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

23. A method comprising delivering to eukaryotic cells

(a) a first vector comprising (i) a N-terminal fragment of a bsr gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the bsr gene and (ii) a second molecule of interest,

wherein the N-terminal fragment and the C-terminal fragment of the intein catalyze joining of a protein fragment encoded by the N-terminal fragment of the bsr gene to a protein fragment encoded by the C-terminal fragment of the bsr gene to produce full-length blasticidin-S deaminase.

24. The method of paragraph 23, wherein the protein fragment encoded by the N-terminal fragment of the bsr gene comprises an amino acid sequence identified by amino acids 1-102 of SEQ ID NO: 4, and the protein fragment encoded by the C-terminal fragment of the bsr gene comprises an amino acid sequence identified by amino acids 103-140 of SEQ ID NO: 4.
25. The method of paragraph 22 or 23, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

26. A method comprising delivering to eukaryotic cells

(a) a first vector comprising (i) a N-terminal fragment of a pac gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the pac gene and (ii) a second molecule of interest,

wherein the N-terminal fragment and the C-terminal fragment of the intein catalyze joining of a protein fragment encoded by the N-terminal fragment of the pac gene to a protein fragment encoded by the C-terminal fragment of the pac gene to produce full-length puromycin N-acetyl-transferase.
27. The method of paragraph 26, wherein

the protein fragment encoded by the N-terminal fragment of the pac gene comprises an amino acid sequence identified by amino acids 1-63 of SEQ ID NO: 2, and the protein fragment encoded by the C-terminal fragment of the pac gene comprises an amino acid sequence identified by amino acids 64-199 of SEQ ID NO: 2;

the protein fragment encoded by the N-terminal fragment of the pac gene comprises an amino acid sequence identified by amino acids 1-119 of SEQ ID NO: 2, and the protein fragment encoded by the C-terminal fragment of the pac gene comprises an amino acid sequence identified by amino acids 120-199 of SEQ ID NO: 2;

the protein fragment encoded by the N-terminal fragment of the pac gene comprises an amino acid sequence identified by amino acids 1-100 of SEQ ID NO: 2, and the protein fragment encoded by the C-terminal fragment of the pac gene comprises an amino acid sequence identified by amino acids 101-199 of SEQ ID NO: 2.

28. The method of paragraph 26 or 27, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

29. A method comprising delivering to eukaryotic cells

(a) a first vector comprising (i) a N-terminal fragment of a neo gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the neo gene and (ii) a second molecule of interest,

wherein the N-terminal fragment and the C-terminal fragment of the intein catalyze joining of a protein fragment encoded by the N-terminal fragment of the neo gene to a protein fragment encoded by the C-terminal fragment of the neo gene to produce full-length aminoglycoside 3′-phosphotransferase.

30. The method of paragraph 29, wherein

the protein fragment encoded by the N-terminal fragment of the neo gene comprises an amino acid sequence identified by amino acids 1-133 of SEQ ID NO: 3 and the protein fragment encoded by the C-terminal fragment of the neo gene comprises an amino acid sequence identified by amino acids 134-267 of SEQ ID NO: 3; or

the protein fragment encoded by the N-terminal fragment of the neo gene comprises an amino acid sequence identified by amino acids 1-194 of SEQ ID NO: 3 and the protein fragment encoded by the C-terminal fragment of the neo gene comprises an amino acid sequence identified by amino acids 195-267 of SEQ ID NO: 3.

31. The method of paragraph 29 or 30, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

32. A method comprising delivering to a composition comprising eukaryotic cells

(a) a first vector comprising (i) a nucleotide sequence encoding an N-terminal fragment of a fluorescent protein, which is upstream from a nucleotide sequence encoding an N-terminal fragment of an intein and (ii) a nucleotide sequence encoding a first molecule of interest; and

(b) a second vector comprising (ii) a nucleotide sequence encoding a C-terminal fragment of the intein, which is upstream from a C-terminal fragment of the fluorescent protein and (ii) a nucleotide sequence encoding a second molecule of interest,

wherein the N-terminal fragment and the C-terminal fragment of the intein catalyze joining of the N-terminal fragment of the fluorescent protein to the C-terminal fragment of the fluorescent protein to produce a full-length fluorescent protein.

33. The method of paragraph 51 further comprising maintaining the eukaryotic cells under conditions that permit introduction of the first and second vectors into the eukaryotic cells to produce transgenic eukaryotic cells.
34. The method of paragraph 33 further comprising selecting the transgenic eukaryotic cells that comprise the full-length fluorescent protein.
35. The method of any one of paragraphs 32-34, wherein the eukaryotic cells are mammalian cells.
36. The method of any one of paragraphs 32-35, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
37. The method of any one of paragraphs 32-36, wherein the intein is a split intein.
38. The method of paragraph 37, wherein the split intein is a natural split intein.
39. The method of paragraph 38, wherein the natural split intein is selected from DnaE inteins.
40. The method of paragraph 39, wherein the DnaE inteins are selected from Synechocystis sp. DnaE (SspDnaE) inteins and Nostoc punctiforme (NpuDnaE) inteins.
41. The method of paragraph 40, wherein the split intein is an engineered split intein.
42. The method of paragraph 41, wherein the engineered split intein is engineered from DnaB inteins.
43. The method of paragraph 42, wherein the engineered split intein is a SspDnaB S1 intein.
44. The method of paragraph 42, wherein the engineered split intein is engineered from GyrB inteins.
45. The method of paragraph 44, wherein the engineered split intein is a SspGyrB S11 intein.
46. The method of any one of paragraphs 32-45, wherein the first and/or second molecule is a protein.
47. The method of any one of paragraphs 32-46, wherein the first and/or second molecule is a non-coding ribonucleic acid (RNA).
48. The method of paragraph 47, wherein the non-coding RNA is a microRNA (miRNA), antisense RNA, short-interfering RNA (siRNA) or short-hairpin RNA (shRNA).
49. The method of any one of paragraphs 32-48, wherein the first and/or second vector is a plasmid vector or a viral vector.
50. A method comprising delivering to eukaryotic cells

(a) a first vector comprising (i) an N-terminal fragment of an egfp gene, which is upstream from a nucleotide sequence encoding an N-terminal fragment of an intein and (ii) a first molecule of interest; and

(b) a second vector comprising (ii) a nucleotide sequence encoding a C-terminal fragment of an intein, which is upstream from a C-terminal fragment of an egfp gene and (ii) a second molecule of interest,

wherein the N-terminal fragment and the C-terminal fragment of the intein catalyze joining of a protein fragment encoded by the N-terminal fragment of the egfp gene to a protein fragment encoded by the C-terminal fragment of the egfp gene to produce full-length EGFP protein.

51. The method of paragraph 50, wherein the protein fragment encoded by the N-terminal fragment of the egfp gene comprises an amino acid sequence identified by amino acids 1-175 of SEQ ID NO: 5, and the protein fragment encoded by the C-terminal fragment of the egfp gene comprises an amino acid sequence identified by amino acids 175-239 of SEQ ID NO: 5.
52. The method of paragraph 50 or 51, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

53. A method comprising delivering to eukaryotic cells

(a) a first vector comprising (i) an N-terminal fragment of an mScarlet gene, which is upstream from a nucleotide sequence encoding an N-terminal fragment of an intein and (ii) a first molecule of interest; and

(b) a second vector comprising (ii) a nucleotide sequence encoding a C-terminal fragment of an intein, which is upstream from a C-terminal fragment of an mScarlet gene and (ii) a second molecule of interest,

wherein the N-terminal fragment and the C-terminal fragment of the intein catalyze joining of a protein fragment encoded by the N-terminal fragment of the mScarlet gene to a protein fragment encoded by the C-terminal fragment of the mScarlet gene to produce full-length mScarlet protein.

54. The method of paragraph 53, wherein

the protein fragment encoded by the N-terminal fragment of the mScarlet gene comprises an amino acid sequence identified by amino acids 1-46 of SEQ ID NO: 6, and the protein fragment encoded by the C-terminal fragment of the mScarlet gene comprises an amino acid sequence identified by amino acids 47-232 of SEQ ID NO: 6;

the protein fragment encoded by the N-terminal fragment of the mScarlet gene comprises an amino acid sequence identified by amino acids 1-48 of SEQ ID NO: 6, and the protein fragment encoded by the C-terminal fragment of the mScarlet gene comprises an amino acid sequence identified by amino acids 49-232 of SEQ ID NO: 6;

the protein fragment encoded by the N-terminal fragment of the mScarlet gene comprises an amino acid sequence identified by amino acids 1-51 of SEQ ID NO: 6, and the protein fragment encoded by the C-terminal fragment of the mScarlet gene comprises an amino acid sequence identified by amino acids 52-232 of SEQ ID NO: 6;

the protein fragment encoded by the N-terminal fragment of the mScarlet gene comprises an amino acid sequence identified by amino acids 1-75 of SEQ ID NO: 6, and the protein fragment encoded by the C-terminal fragment of the mScarlet gene comprises an amino acid sequence identified by amino acids 76-232 of SEQ ID NO: 6;

the protein fragment encoded by the N-terminal fragment of the mScarlet gene comprises an amino acid sequence identified by amino acids 1-122 of SEQ ID NO: 6, and the protein fragment encoded by the C-terminal fragment of the mScarlet gene comprises an amino acid sequence identified by amino acids 123-232 of SEQ ID NO: 6;

the protein fragment encoded by the N-terminal fragment of the mScarlet gene comprises an amino acid sequence identified by amino acids 1-140 of SEQ ID NO: 6, and the protein fragment encoded by the C-terminal fragment of the mScarlet gene comprises an amino acid sequence identified by amino acids 141-232 of SEQ ID NO: 6;

the protein fragment encoded by the N-terminal fragment of the mScarlet gene comprises an amino acid sequence identified by amino acids 1-163 of SEQ ID NO: 6, and the protein fragment encoded by the C-terminal fragment of the mScarlet gene comprises an amino acid sequence identified by amino acids 164-232 of SEQ ID NO: 6.

55. The method of paragraph 53 or 54, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

56. A eukaryotic cell, comprising

(a) a first vector comprising (i) a nucleotide sequence encoding an N-terminal fragment of an antibiotic resistance protein, which is upstream from a nucleotide sequence encoding an N-terminal fragment of an intein and (ii) a nucleotide sequence encoding a first molecule of interest; and

(b) a second vector comprising (i) a nucleotide sequence encoding a C-terminal fragment of the intein, which is upstream from a C-terminal fragment of the antibiotic resistance protein and (ii) a nucleotide sequence encoding a second molecule of interest,

wherein the N-terminal fragment and the C-terminal fragment of the intein catalyze joining of the N-terminal fragment and the C-terminal fragment of the antibiotic resistance protein to produce a full-length antibiotic resistance protein.

57. The cell of paragraph 56, wherein the eukaryotic cells are mammalian cells.
58. The cell of paragraph 56 or 57, wherein the antibiotic resistance protein confers resistance to hygromycin, G418, puromycin, phleomycin D1 or blasticidin.
59. The cell of any one of paragraphs 56-58, wherein the intein is a split intein.
60. The cell of paragraph 59, wherein the split intein is a natural split intein.
61. The cell of paragraph 60, wherein the natural split intein is selected from DnaE inteins.
62. The cell of paragraph 61, wherein the DnaE inteins are selected from Synechocystis sp. DnaE (SspDnaE) inteins and Nostoc punctiforme (NpuDnaE) inteins.
63. The cell of paragraph 59, wherein the split intein is an engineered split intein.
64. The cell of paragraph 63, wherein the engineered split intein is engineered from DnaB inteins.
65. The cell of paragraph 64, wherein the engineered split intein is a SspDnaB S1 intein.
66. The cell of paragraph 65, wherein the engineered split intein is engineered from GyrB inteins.
67. The cell of paragraph 66, wherein the engineered split intein is a SspGyrB S11 intein.
68. The cell of any one of paragraphs 56-67, wherein the first and/or second molecule is a protein.
69. The cell of any one of paragraphs 56-68, wherein the first and/or second molecule is a non-coding ribonucleic acid (RNA).
70. The cell of paragraph 69, wherein the non-coding RNA is a microRNA (miRNA), antisense RNA, short-interfering RNA (siRNA) or short-hairpin RNA (shRNA).
71. The cell of any one of paragraphs 56-70, wherein the first and/or second vector is a plasmid vector or a viral vector.
72. A cell comprising

(a) a first vector comprising (i) an N-terminal fragment of a hygB gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the hygB gene and (ii) a second molecule of interest,

wherein the N-terminal fragment and C-terminal fragment of the intein catalyze joining of a protein fragment encoded by the N-terminal fragment of the hygB gene to a protein fragment encoded by the C-terminal fragment of the hygB gene to produce full-length hygromycin B phosphotransferase.

73. The cell of paragraph 72, wherein the first amino acid of the protein fragment encoded by the second hygB gene fragment is cysteine.
74. The cell of paragraph 73, wherein

the protein fragment encoded by the N-terminal fragment of the hygB gene comprises an amino acid sequence identified by amino acids 1-89 of SEQ ID NO: 1, and the protein fragment encoded by the C-terminal fragment of the hygB gene comprises an amino acid sequence identified by amino acids 90-341 of SEQ ID NO: 1;

the protein fragment encoded by the N-terminal fragment of the hygB gene comprises an amino acid sequence identified by amino acids 1-200 of SEQ ID NO: 1, and the protein fragment encoded by the C-terminal fragment of the hygB gene comprises an amino acid sequence identified by amino acids 201-341 of SEQ ID NO: 1;

the protein fragment encoded by the N-terminal fragment of the hygB gene comprises an amino acid sequence identified by amino acids 1-53 of SEQ ID NO: 1, and the protein fragment encoded by the C-terminal fragment of the hygB gene comprises an amino acid sequence identified by amino acids 54-341 of SEQ ID NO: 1;

the protein fragment encoded by the N-terminal fragment of the hygB gene comprises an amino acid sequence identified by amino acids 1-240 of SEQ ID NO: 1, and the protein fragment encoded by the C-terminal fragment of the hygB gene comprises an amino acid sequence identified by amino acids 241-341 of SEQ ID NO: 1;

the protein fragment encoded by the N-terminal fragment of the hygB gene comprises an amino acid sequence identified by amino acids 1-292 of SEQ ID NO: 1, and the protein fragment encoded by the C-terminal fragment of the hygB gene comprises an amino acid sequence identified by amino acids 293-341 of SEQ ID NO: 1.

75. The cell of any one of paragraphs 72-74, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

76. A eukaryotic cell, comprising

(a) a first vector comprising (i) a N-terminal fragment of a bsr gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the bsr gene and (ii) a second molecule of interest,

wherein the N-terminal fragment and the C-terminal fragment of the intein catalyze joining of a protein fragment encoded by the N-terminal fragment of the bsr gene to a protein fragment encoded by the C-terminal fragment of the bsr gene to produce full-length blasticidin-S deaminase.

77. The cell of paragraph 76, wherein the protein fragment encoded by the N-terminal fragment of the bsr gene comprises an amino acid sequence identified by amino acids 1-102 of SEQ ID NO: 4, and the protein fragment encoded by the C-terminal fragment of the bsr gene comprises an amino acid sequence identified by amino acids 103-140 of SEQ ID NO: 4.
78. The cell of paragraph 76 or 77, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

79. A eukaryotic cell, comprising

(a) a first vector comprising (i) a N-terminal fragment of a pac gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the pac gene and (ii) a second molecule of interest,

wherein the N-terminal fragment and the C-terminal fragment of the intein catalyze joining of a protein fragment encoded by the N-terminal fragment of the pac gene to a protein fragment encoded by the C-terminal fragment of the pac gene to produce full-length puromycin N-acetyl-transferase.

80. The cell of paragraph 79, wherein

the protein fragment encoded by the N-terminal fragment of the pac gene comprises an amino acid sequence identified by amino acids 1-63 of SEQ ID NO: 2, and the protein fragment encoded by the C-terminal fragment of the pac gene comprises an amino acid sequence identified by amino acids 64-199 of SEQ ID NO: 2;

the protein fragment encoded by the N-terminal fragment of the pac gene comprises an amino acid sequence identified by amino acids 1-119 of SEQ ID NO: 2, and the protein fragment encoded by the C-terminal fragment of the pac gene comprises an amino acid sequence identified by amino acids 120-199 of SEQ ID NO: 2;

the protein fragment encoded by the N-terminal fragment of the pac gene comprises an amino acid sequence identified by amino acids 1-100 of SEQ ID NO: 2, and the protein fragment encoded by the C-terminal fragment of the pac gene comprises an amino acid sequence identified by amino acids 101-199 of SEQ ID NO: 2.

81. The cell of paragraph 79 or 80, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

82. A eukaryotic cell, comprising

(a) a first vector comprising (i) a N-terminal fragment of a neo gene, which is upstream from a nucleotide sequence encoding an N-terminal fragment of an intein and (ii) a first molecule of interest; and

(b) a second vector comprising (ii) a nucleotide sequence encoding a C-terminal fragment of the intein, which is upstream from a C-terminal fragment of the neo gene and (ii) a second molecule of interest,

wherein the N-terminal fragment and the C-terminal fragment of the intein catalyze joining of a protein fragment encoded by the N-terminal fragment of the neo gene to a protein fragment encoded by the C-terminal fragment of the neo gene to produce full-length aminoglycoside 3′-phosphotransferase.

83. The cell of paragraph 82, wherein

the protein fragment encoded by the N-terminal fragment of the neo gene comprises an amino acid sequence identified by amino acids 1-133 of SEQ ID NO: 3 and the protein fragment encoded by the C-terminal fragment of the neo gene comprises an amino acid sequence identified by amino acids 134-267 of SEQ ID NO: 3; or

the protein fragment encoded by the N-terminal fragment of the neo gene comprises an amino acid sequence identified by amino acids 1-194 of SEQ ID NO: 3 and the protein fragment encoded by the C-terminal fragment of the neo gene comprises an amino acid sequence identified by amino acids 195-267 of SEQ ID NO: 3.

84. The cell of paragraph 82 or 83, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

85. A eukaryotic cell, comprising

(a) a first vector comprising (i) a nucleotide sequence encoding an N-terminal fragment of a fluorescent protein, which is upstream from a nucleotide sequence encoding an N-terminal fragment of an intein and (ii) a nucleotide sequence encoding a first molecule of interest; and

(b) a second vector comprising (ii) a nucleotide sequence encoding a C-terminal fragment of the intein, which is upstream from a C-terminal fragment of the fluorescent protein and (ii) a nucleotide sequence encoding a second molecule of interest,

wherein the N-terminal fragment and the C-terminal fragment of the intein catalyze joining of the N-terminal fragment of the fluorescent protein to the C-terminal fragment of the fluorescent protein to produce a full-length fluorescent protein.

86. The cell of paragraph 85 further comprising maintaining the eukaryotic cells under conditions that permit introduction of the first and second vectors into the eukaryotic cells to produce transgenic eukaryotic cells.
87. The cell of paragraph 86 further comprising selecting the transgenic eukaryotic cells that comprise the full-length fluorescent protein.
88. The cell of any one of paragraphs 85-87, wherein the eukaryotic cells are mammalian cells.
89. The cell of any one of paragraphs 85-88, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
90. The cell of any one of paragraphs 85-89, wherein the intein is a split intein.
91. The cell of paragraph 90, wherein the split intein is a natural split intein.
92. The cell of paragraph 91, wherein the natural split intein is selected from DnaE inteins.
93. The cell of paragraph 92, wherein the DnaE inteins are selected from Synechocystis sp. DnaE (SspDnaE) inteins and Nostoc punctiforme (NpuDnaE) inteins.
94. The cell of paragraph 93, wherein the split intein is an engineered split intein.
95. The cell of paragraph 94, wherein the engineered split intein is engineered from DnaB inteins.
96. The cell of paragraph 95, wherein the engineered split intein is a SspDnaB S1 intein.
97. The cell of paragraph 95, wherein the engineered split intein is engineered from GyrB inteins.
98. The cell of paragraph 97, wherein the engineered split intein is a SspGyrB S11 intein.
99. The cell of any one of paragraphs 85-98, wherein the first and/or second molecule is a protein.
100. The cell of any one of paragraphs 85-99, wherein the first and/or second molecule is a non-coding ribonucleic acid (RNA).
101. The cell of paragraph 100, wherein the non-coding RNA is a microRNA (miRNA), antisense RNA, short-interfering RNA (siRNA) or short-hairpin RNA (shRNA).
102. The cell of any one of paragraphs 85-101, wherein the first and/or second vector is a plasmid vector or a viral vector.
103. A eukaryotic cell, comprising

(a) a first vector comprising (i) an N-terminal fragment of an egfp gene, which is upstream from a nucleotide sequence encoding an N-terminal fragment of an intein and (ii) a first molecule of interest; and

(b) a second vector comprising (ii) a nucleotide sequence encoding a C-terminal fragment of an intein, which is upstream from a C-terminal fragment of an egfp gene and (ii) a second molecule of interest,

wherein the N-terminal fragment and the C-terminal fragment of the intein catalyze joining of a protein fragment encoded by the N-terminal fragment of the egfp gene to a protein fragment encoded by the C-terminal fragment of the egfp gene to produce full-length EGFP protein.

104. The cell of paragraph 103, wherein the protein fragment encoded by the N-terminal fragment of the egfp gene comprises an amino acid sequence identified by amino acids 1-175 of SEQ ID NO: 5, and the protein fragment encoded by the C-terminal fragment of the egfp gene comprises an amino acid sequence identified by amino acids 175-239 of SEQ ID NO: 5.
105. The cell of paragraph 103 or 104, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

106. A eukaryotic cell, comprising

(a) a first vector comprising (i) an N-terminal fragment of an mScarlet gene, which is upstream from a nucleotide sequence encoding an N-terminal fragment of an intein and (ii) a first molecule of interest; and

(b) a second vector comprising (ii) a nucleotide sequence encoding a C-terminal fragment of an intein, which is upstream from a C-terminal fragment of an mScarlet gene and (ii) a second molecule of interest,

wherein the N-terminal fragment and the C-terminal fragment of the intein catalyze joining of a protein fragment encoded by the N-terminal fragment of the mScarlet gene to a protein fragment encoded by the C-terminal fragment of the mScarlet gene to produce full-length mScarlet protein.

107. The cell of paragraph 106, wherein

the protein fragment encoded by the N-terminal fragment of the mScarlet gene comprises an amino acid sequence identified by amino acids 1-46 of SEQ ID NO: 6, and the protein fragment encoded by the C-terminal fragment of the mScarlet gene comprises an amino acid sequence identified by amino acids 47-232 of SEQ ID NO: 6;

the protein fragment encoded by the N-terminal fragment of the mScarlet gene comprises an amino acid sequence identified by amino acids 1-48 of SEQ ID NO: 6, and the protein fragment encoded by the C-terminal fragment of the mScarlet gene comprises an amino acid sequence identified by amino acids 49-232 of SEQ ID NO: 6;

the protein fragment encoded by the N-terminal fragment of the mScarlet gene comprises an amino acid sequence identified by amino acids 1-51 of SEQ ID NO: 6, and the protein fragment encoded by the C-terminal fragment of the mScarlet gene comprises an amino acid sequence identified by amino acids 52-232 of SEQ ID NO: 6;

the protein fragment encoded by the N-terminal fragment of the mScarlet gene comprises an amino acid sequence identified by amino acids 1-75 of SEQ ID NO: 6, and the protein fragment encoded by the C-terminal fragment of the mScarlet gene comprises an amino acid sequence identified by amino acids 76-232 of SEQ ID NO: 6;
the protein fragment encoded by the N-terminal fragment of the mScarlet gene comprises an amino acid sequence identified by amino acids 1-122 of SEQ ID NO: 6, and the protein fragment encoded by the C-terminal fragment of the mScarlet gene comprises an amino acid sequence identified by amino acids 123-232 of SEQ ID NO: 6;

the protein fragment encoded by the N-terminal fragment of the mScarlet gene comprises an amino acid sequence identified by amino acids 1-140 of SEQ ID NO: 6, and the protein fragment encoded by the C-terminal fragment of the mScarlet gene comprises an amino acid sequence identified by amino acids 141-232 of SEQ ID NO: 6;

the protein fragment encoded by the N-terminal fragment of the mScarlet gene comprises an amino acid sequence identified by amino acids 1-163 of SEQ ID NO: 6, and the protein fragment encoded by the C-terminal fragment of the mScarlet gene comprises an amino acid sequence identified by amino acids 164-232 of SEQ ID NO: 6.

108. The cell of paragraph 106 or 107, wherein

the N-terminal fragment of the intein is identified by SEQ ID NO:16, and the C-terminal fragment of the intein is identified by SEQ ID NO:17;

the N-terminal fragment of the intein is identified by SEQ ID NO:7, and the C-terminal fragment of the intein is identified by SEQ ID NO:8; or

the N-terminal fragment of the intein is identified by SEQ ID NO:18 or SEQ ID NO:9, and the C-terminal fragment of the intein is identified by SEQ ID NO:19 or SEQ ID NO:10.

109. A composition comprising the cell of any one of paragraph 85-108.
110. A kit, comprising

(a) a first vector comprising a nucleotide sequence encoding an N-terminal fragment of an antibiotic resistance protein, which is upstream from a nucleotide sequence encoding an N-terminal fragment of an intein; and

(b) a second vector comprising a nucleotide sequence encoding a C-terminal fragment of the intein, which is upstream from a C-terminal fragment of the antibiotic resistance protein,

wherein the N-terminal fragment and the C-terminal fragment of the intein catalyze joining of the N-terminal fragment and the C-terminal fragment of the antibiotic resistance protein to produce a full-length antibiotic resistance protein.

111. The kit of paragraph 110, wherein the antibiotic resistance protein confers resistance to hygromycin, G418, puromycin, phleomycin D1 or blasticidin.
112. A kit, comprising

(a) a first vector comprising a nucleotide sequence encoding an N-terminal fragment of a fluorescent protein, which is upstream from a nucleotide sequence encoding an N-terminal fragment of an intein; and

(b) a second vector comprising a nucleotide sequence encoding a C-terminal fragment of the intein, which is upstream from a C-terminal fragment of the fluorescent protein,

wherein the N-terminal fragment and the C-terminal fragment of the intein catalyze joining of the N-terminal fragment of the fluorescent protein to the C-terminal fragment of the fluorescent protein to produce a full-length fluorescent protein.

113. The kit of paragraph 112, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
114. The kit of any one of paragraphs 110-113, wherein the intein is a split intein.
115. The kit of paragraph 114, wherein the split intein is a natural split intein or an engineered split intein.
116. The kit of paragraph 115, wherein the natural split intein is selected from DnaE inteins.
117. The kit of paragraph 116, wherein the DnaE inteins are selected from Synechocystis sp. DnaE (SspDnaE) inteins and Nostoc punctiforme (NpuDnaE) inteins.
118. The kit of paragraph 115, wherein the engineered split intein is engineered from DnaB inteins or GyrB inteins.
119. The kit of paragraph 118, wherein the engineered split intein is a SspDnaB S1 intein.
120. The kit of paragraph 118, wherein the engineered split intein is a SspGyrB S11 intein.
121. The kit of any one of paragraphs 112-120, further comprising any one or more of the following components: buffers, salts, cloning enzymes, competent cells, transfection reagents, antibiotics, and/or instructions for performing the methods described herein.
122. A method comprising delivering to eukaryotic cells

(a) a first vector comprising (i) a nucleotide sequence encoding an N-terminal fragment of an antibiotic resistance protein, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a nucleotide sequence encoding a C-terminal fragment of the antibiotic resistance protein and (ii) a nucleotide sequence encoding a third molecule of interest,

wherein the N-terminal fragment and the C-terminal fragment of the first intein catalyze joining of N-terminal fragment of the antibiotic resistance protein to the central fragment of the antibiotic resistance protein, and the N-terminal fragment and the C-terminal fragment of the second intein catalyze joining of central fragment of the antibiotic resistance protein to the C-terminal fragment of the antibiotic resistance protein, to produce a full-length antibiotic resistance protein.

123. The method of paragraph 112 further comprising maintaining the eukaryotic cells under conditions that permit introduction of the first, second, and third vectors into the eukaryotic cells to produce transgenic eukaryotic cells.
124. The method of paragraph 123 further comprising selecting the transgenic eukaryotic cells that comprise the full-length antibiotic resistance protein.
125. The method of any one of paragraphs 112-124, wherein the eukaryotic cells are mammalian cells.
126. The method of any one of paragraphs 112-125, wherein the antibiotic resistance protein confers resistance to hygromycin, G418, puromycin, phleomycin D1 or blasticidin.
127. The method of paragraph 126, wherein the antibiotic resistance protein confers resistance to hygromycin.
128. The method of any one of paragraphs 112-127, wherein the first intein is a split intein.
129. The method of any one of paragraphs 112-128, wherein the second intein is a split intein.
130. The method of paragraph 128 or 129, wherein the split intein is a natural split intein.
131. The method of paragraph 130, wherein the natural split intein is selected from DnaE inteins.
132. The method of paragraph 131, wherein the DnaE inteins are selected from Synechocystis sp. DnaE (SspDnaE) inteins and Nostoc punctiforme (NpuDnaE) inteins.
133. The method of paragraph 132, wherein the first intein is an NpuDnaE intein and the second intein is an NpuDnaE intein.
134. The method of any one of paragraphs 112-133, wherein the first molecule of interest, second molecule of interest, third molecule of interest, or any combination thereof is a protein.
135. The method of any one of paragraphs 112-133, wherein the first molecule of interest, second molecule of interest, third molecule of interest, or any combination thereof is a non-coding ribonucleic acid (RNA).
136. The method of paragraph 135, wherein the non-coding RNA is a microRNA (miRNA), antisense RNA, short-interfering RNA (siRNA) or short-hairpin RNA (shRNA).
137. The method of any one of paragraphs 112-136, wherein the first vector, second vector, third vector, or any combination thereof, is a plasmid vector or a viral vector.
138. A method comprising delivering to eukaryotic cells

(a) a first vector comprising (i) an N-terminal fragment of a hygB gene, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a central fragment of the hygB gene, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a C-terminal fragment of the hygB gene and (ii) a nucleotide sequence encoding a third molecule of interest,

wherein the N-terminal fragment and the C-terminal fragment of the first intein catalyze joining of the protein fragment encoded by N-terminal fragment of the hygB gene to a protein fragment encoded by the central fragment of the hygB gene, and the N-terminal fragment and the C-terminal fragment of the second intein catalyze joining of the protein fragment encoded by the central fragment of the hygB gene to the protein fragment encoded by the C-terminal fragment of the hygB gene, to produce a full-length hygromycin B phosphotransferase.

139. The method of paragraph 138, wherein the first vector encodes the sequence identified by SEQ ID NO: 29, the second vector encodes the sequence identified by SEQ ID NO: 61, and the third vector encodes the sequence identified by SEQ ID NO: 23.
140. The method of paragraph 138, wherein the first vector encodes the sequence identified by SEQ ID NO: 21, the second vector encodes the sequence identified by SEQ ID NO: 61, and the third vector encodes the sequence identified by SEQ ID NO: 35.
141. A eukaryotic cell comprising:

(a) a first vector comprising (i) a nucleotide sequence encoding an N-terminal fragment of an antibiotic resistance protein, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a nucleotide sequence encoding a C-terminal fragment of the antibiotic resistance protein and (ii) a nucleotide sequence encoding a third molecule of interest,

wherein the N-terminal fragment and the C-terminal fragment of the first intein catalyze joining of N-terminal fragment of the antibiotic resistance protein to the central fragment of the antibiotic resistance protein, and the N-terminal fragment and the C-terminal fragment of the second intein catalyze joining of central fragment of the antibiotic resistance protein to the C-terminal fragment of the antibiotic resistance protein, to produce a full-length antibiotic resistance protein.

142. The eukaryotic cell of paragraph 112, wherein the eukaryotic cells are mammalian cells.
143. The eukaryotic cell of paragraph 141 or 142, wherein the antibiotic resistance protein confers resistance to hygromycin, G418, puromycin, phleomycin D1 or blasticidin.
144. The eukaryotic cell of paragraph 143, wherein the antibiotic resistance protein confers resistance to hygromycin.
145. The eukaryotic cell of any one of paragraphs 141-144, wherein the first intein is a split intein.
146. The eukaryotic cell of any one of paragraphs 142-145, wherein the second intein is a split intein.
147. The eukaryotic cell of paragraph 145 or 146, wherein the split intein is a natural split intein.
148. The eukaryotic cell of paragraph 147, wherein the natural split intein is selected from DnaE inteins.
149. The eukaryotic cell of paragraph 148, wherein the DnaE inteins are selected from Synechocystis sp. DnaE (SspDnaE) inteins and Nostoc punctiforme (NpuDnaE) inteins.
150. The eukaryotic cell of paragraph 149, wherein the first intein is an NpuDnaE intein and the second intein is an NpuDnaE intein.
151. The eukaryotic cell of any one of paragraphs 142-150, wherein the first molecule of interest, second molecule of interest, third molecule of interest, or any combination thereof is a protein.
152. The eukaryotic cell of any one of paragraphs 142-150, wherein the first molecule of interest, second molecule of interest, third molecule of interest, or any combination thereof is a non-coding ribonucleic acid (RNA).
153. The eukaryotic cell of paragraph 152, wherein the non-coding RNA is a microRNA (miRNA), antisense RNA, short-interfering RNA (siRNA) or short-hairpin RNA (shRNA).
154. The eukaryotic cell of any one of paragraphs 142-153, wherein the first vector, second vector, third vector, or any combination thereof, is a plasmid vector or a viral vector.
155. A composition comprising the eukaryotic cell of any one of paragraph 142-154.
156. A kit comprising:

(a) a first vector comprising a nucleotide sequence encoding an N-terminal fragment of an antibiotic resistance protein, which is upstream from a nucleotide sequence encoding an N-terminal fragment of a first intein,

(b) a second vector comprising a nucleotide sequence encoding a C-terminal fragment of the first intein, which is upstream from a nucleotide sequence encoding a central fragment of the antibiotic resistance protein, which is upstream from a nucleotide sequence encoding an N-terminal fragment of a second intein, and

(c) a third vector comprising a nucleotide sequence encoding a C-terminal fragment of the second intein, which is upstream from a nucleotide sequence encoding a C-terminal fragment of the antibiotic resistance protein,

wherein the N-terminal fragment and the C-terminal fragment of the first intein catalyze joining of N-terminal fragment of the antibiotic resistance protein to the central fragment of the antibiotic resistance protein, and the N-terminal fragment and the C-terminal fragment of the second intein catalyze joining of central fragment of the antibiotic resistance protein to the C-terminal fragment of the antibiotic resistance protein, to produce a full-length antibiotic resistance protein.

157. The kit of paragraph 156, wherein the antibiotic resistance protein confers resistance to hygromycin, G418, puromycin, phleomycin D1 or blasticidin.
158. The kit of paragraph 157, wherein the antibiotic resistance protein confers resistance to hygromycin.
159. The kit of any one of paragraphs 156-158, wherein the first intein is a split intein.
160. The kit of any one of paragraphs 156-159, wherein the second intein is a split intein.
161. The kit of paragraph 159 or 160, wherein the split intein is a natural split intein.
162. The kit of paragraph 161, wherein the natural split intein is selected from DnaE inteins.
163. The kit of paragraph 162, wherein the DnaE inteins are selected from Synechocystis sp. DnaE (SspDnaE) inteins and Nostoc punctiforme (NpuDnaE) inteins.
164. The kit of paragraph 163, wherein the first intein is an NpuDnaE intein and the second intein is an NpuDnaE intein.
165. The kit of any one of paragraphs 156-164, wherein the first molecule of interest, second molecule of interest, third molecule of interest, or any combination thereof is a protein.
166. The kit of any one of paragraphs 156-164, wherein the first molecule of interest, second molecule of interest, third molecule of interest, or any combination thereof is a non-coding ribonucleic acid (RNA).
167. The kit of paragraph 166, wherein the non-coding RNA is a microRNA (miRNA), antisense RNA, short-interfering RNA (siRNA) or short-hairpin RNA (shRNA).
168. The kit of any one of paragraphs 156-167, wherein the first vector, second vector, third vector, or any combination thereof, is a plasmid vector or a viral vector.
169. A method comprising delivering to eukaryotic cells

(a) a first vector comprising (i) a nucleotide sequence encoding an N-terminal fragment of a fluorescent protein, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a nucleotide sequence encoding a central fragment of the fluorescent protein, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a nucleotide sequence encoding a C-terminal fragment of the fluorescent protein and (ii) a nucleotide sequence encoding a third molecule of interest,

wherein the N-terminal fragment and the C-terminal fragment of the first intein catalyze joining of N-terminal fragment of the fluorescent protein to the central fragment of the fluorescent protein, and the N-terminal fragment and the C-terminal fragment of the second intein catalyze joining of central fragment of the fluorescent protein to the C-terminal fragment of the fluorescent protein, to produce a full-length fluorescent protein.

170. The method of paragraph 169 further comprising maintaining the eukaryotic cells under conditions that permit introduction of the first, second, and third vectors into the eukaryotic cells to produce transgenic eukaryotic cells.
171. The method of paragraph 170 further comprising selecting the transgenic eukaryotic cells that comprise the full-length fluorescent protein.
172. The method of any one of paragraphs 169-171, wherein the eukaryotic cells are mammalian cells.
173. The method of any one of paragraphs 169-172, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mScarlet, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
174. The method of paragraph 173, wherein the fluorescent protein is mScarlet.
175. The method of any one of paragraphs 169-174, wherein the first intein is a split intein.
176. The method of any one of paragraphs 169-175, wherein the second intein is a split intein.
177. The method of paragraph 175 or 176, wherein the split intein is a natural split intein.
178. The method of paragraph 177, wherein the natural split intein is selected from DnaE inteins.
179. The method of paragraph 178, wherein the DnaE inteins are selected from Synechocystis sp. DnaE (SspDnaE) inteins and Nostoc punctiforme (NpuDnaE) inteins.
180. The method of paragraph 179, wherein the first intein is an NpuDnaE intein and the second intein is an NpuDnaE intein.
181. The method of any one of paragraphs 169-170, wherein the first molecule of interest, second molecule of interest, third molecule of interest, or any combination thereof is a protein.
182. The method of any one of paragraphs 169-180, wherein the first molecule of interest, second molecule of interest, third molecule of interest, or any combination thereof is a non-coding ribonucleic acid (RNA).
183. The method of paragraph 182, wherein the non-coding RNA is a microRNA (miRNA), antisense RNA, short-interfering RNA (siRNA) or short-hairpin RNA (shRNA).
184. The method of any one of paragraphs 169-183, wherein the first vector, second vector, third vector, or any combination thereof, is a plasmid vector or a viral vector.
185. A method comprising delivering to eukaryotic cells

(a) a first vector comprising (i) an N-terminal fragment of a mScarlet gene, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a central fragment of the mScarlet gene, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a C-terminal fragment of the mScarlet gene and (ii) a nucleotide sequence encoding a third molecule of interest,

wherein the N-terminal fragment and the C-terminal fragment of the first intein catalyze joining of the protein fragment encoded by N-terminal fragment of the mScarlet gene to a protein fragment encoded by the central fragment of the mScarlet gene, and the N-terminal fragment and the C-terminal fragment of the second intein catalyze joining of the protein fragment encoded by the central fragment of the mScarlet gene to the protein fragment encoded by the C-terminal fragment of the mScarlet gene, to produce a full-length mScarlet protein.

186. The method of paragraph 185, wherein the first vector encodes the sequence identified by SEQ ID NO: 121, the second vector encodes the sequence identified by SEQ ID NO: 123, and the third vector encodes the sequence identified by SEQ ID NO: 125.
187. A eukaryotic cell comprising:

(a) a first vector comprising (i) a nucleotide sequence encoding an N-terminal fragment of a fluorescent protein, which is upstream from a nucleotide sequence encoding an N-terminal fragment of a first intein and (ii) a nucleotide sequence encoding a first molecule of interest,

(b) a second vector comprising (i) a nucleotide sequence encoding a C-terminal fragment of the first intein, which is upstream from a nucleotide sequence encoding a central fragment of the fluorescent protein, which is upstream from a nucleotide sequence encoding an N-terminal fragment of a second intein and (ii) a nucleotide sequence encoding a second molecule of interest, and

(c) a third vector comprising (i) a nucleotide sequence encoding a C-terminal fragment of the second intein, which is upstream from a nucleotide sequence encoding a C-terminal fragment of the fluorescent protein and (ii) a nucleotide sequence encoding a third molecule of interest,

wherein the N-terminal fragment and the C-terminal fragment of the first intein catalyze joining of N-terminal fragment of the fluorescent protein to the central fragment of the fluorescent protein, and the N-terminal fragment and the C-terminal fragment of the second intein catalyze joining of central fragment of the fluorescent protein to the C-terminal fragment of the fluorescent protein, to produce a full-length fluorescent protein.

188. The eukaryotic cell of paragraph 187 wherein the eukaryotic cells are mammalian cells.
189. The eukaryotic cell of paragraph 187 or 188, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mScarlet, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
190. The eukaryotic cell of paragraph 189, wherein the fluorescent protein is mScarlet.
191. The eukaryotic cell of any one of paragraphs 187-190, wherein the first intein is a split intein.
192. The eukaryotic cell of any one of paragraphs 185-191, wherein the second intein is a split intein.
193. The eukaryotic cell of paragraph 191 or 192, wherein the split intein is a natural split intein.
194. The eukaryotic cell of paragraph 193, wherein the natural split intein is selected from DnaE inteins.
195. The eukaryotic cell of paragraph 194, wherein the DnaE inteins are selected from Synechocystis sp. DnaE (SspDnaE) inteins and Nostoc punctiforme (NpuDnaE) inteins.
196. The eukaryotic cell of paragraph 195, wherein the first intein is an NpuDnaE intein and the second intein is an NpuDnaE intein.
197. The eukaryotic cell of any one of paragraphs 185-196, wherein the first molecule of interest, second molecule of interest, third molecule of interest, or any combination thereof is a protein.
198. The eukaryotic cell of any one of paragraphs 185-196, wherein the first molecule of interest, second molecule of interest, third molecule of interest, or any combination thereof is a non-coding ribonucleic acid (RNA).
199. The eukaryotic cell of paragraph 198, wherein the non-coding RNA is a microRNA (miRNA), antisense RNA, short-interfering RNA (siRNA) or short-hairpin RNA (shRNA).
200. The eukaryotic cell of any one of paragraphs 185-199, wherein the first vector, second vector, third vector, or any combination thereof, is a plasmid vector or a viral vector.
201. A composition comprising the eukaryotic cell of any one of paragraph 185-200.
202. A kit comprising:

(a) a first vector comprising a nucleotide sequence encoding an N-terminal fragment of a fluorescent protein, which is upstream from a nucleotide sequence encoding an N-terminal fragment of a first intein,

(b) a second vector comprising a nucleotide sequence encoding a C-terminal fragment of the first intein, which is upstream from a nucleotide sequence encoding a central fragment of the fluorescent protein, which is upstream from a nucleotide sequence encoding an N-terminal fragment of a second intein, and

(c) a third vector comprising a nucleotide sequence encoding a C-terminal fragment of the second intein, which is upstream from a nucleotide sequence encoding a C-terminal fragment of the fluorescent protein,

wherein the N-terminal fragment and the C-terminal fragment of the first intein catalyze joining of N-terminal fragment of the fluorescent protein to the central fragment of the fluorescent protein, and the N-terminal fragment and the C-terminal fragment of the second intein catalyze joining of central fragment of the fluorescent protein to the C-terminal fragment of the fluorescent protein, to produce a full-length fluorescent protein.

203. The kit of paragraph 202, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mScarlet, mKO2, mOrange, mOrange2, mRaspberry, mCherry, mStrawberry, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.
204. The kit of paragraph 203, wherein the fluorescent protein is mScarlet.
205. The kit of any one of paragraphs 202-204, wherein the first intein is a split intein.
206. The kit of any one of paragraphs 202-205, wherein the second intein is a split intein.
207. The kit of paragraph 206, wherein the split intein is a natural split intein.
208. The kit of paragraph 207, wherein the natural split intein is selected from DnaE inteins.
209. The kit of paragraph 208, wherein the DnaE inteins are selected from Synechocystis sp. DnaE (SspDnaE) inteins and Nostoc punctiforme (NpuDnaE) inteins.
210. The kit of paragraph 209, wherein the first intein is an NpuDnaE intein and the second intein is an NpuDnaE intein.
211. The kit of any one of paragraphs 202-210, wherein the first molecule of interest, second molecule of interest, third molecule of interest, or any combination thereof is a protein.
212. The kit of any one of paragraphs 202-210, wherein the first molecule of interest, second molecule of interest, third molecule of interest, or any combination thereof is a non-coding ribonucleic acid (RNA).
213. The kit of paragraph 212, wherein the non-coding RNA is a microRNA (miRNA), antisense RNA, short-interfering RNA (siRNA) or short-hairpin RNA (shRNA).
214. The kit of any one of paragraphs 202-213, wherein the first vector, second vector, third vector, or any combination thereof, is a plasmid vector or a viral vector.
215. The kit of any one of paragraphs 202-214, further comprising any one or more of the following components: buffers, salts, cloning enzymes, competent cells, transfection reagents, antibiotics, and/or instructions for performing the methods described herein.
216. A transgenic selection method comprising delivering to a composition comprising eukaryotic cells (a) a first vector comprising (i) a nucleotide sequence encoding a first selectable marker protein fragment (e.g., antibiotic resistance protein fragment or fluorescent protein fragment) upstream from a nucleotide sequence encoding an N-terminal intein protein fragment and (ii) a nucleotide sequence encoding a first molecule, and (b) a second vector comprising (i) a nucleotide sequence encoding a C-terminal intein protein fragment upstream from a second selectable marker protein fragment (e.g., antibiotic resistance protein fragment or fluorescent protein fragment) and (ii) a nucleotide sequence encoding a second molecule, wherein the N-terminal intein protein fragment and the C-terminal intein protein fragment catalyze joining of the first selectable marker protein fragment to the second selectable marker protein fragment to produce a full-length selectable marker protein.
217. A transgenic selection method comprising delivering to eukaryotic cells (a) a first vector comprising (i) a nucleotide sequence encoding an N-terminal fragment of a selectable marker protein (e.g., antibiotic resistance protein or fluorescent protein), which is upstream from a nucleotide sequence encoding an N-terminal fragment of a first intein and (ii) a nucleotide sequence encoding a first molecule of interest, (b) a second vector comprising (i) a nucleotide sequence encoding a C-terminal fragment of the first intein, which is upstream from a nucleotide sequence encoding a central fragment of the selectable marker protein, which is upstream from a nucleotide sequence encoding an N-terminal fragment of a second intein and (ii) a nucleotide sequence encoding a second molecule of interest, and (c) a third vector comprising (i) a nucleotide sequence encoding a C-terminal fragment of the second intein, which is upstream from a nucleotide sequence encoding a C-terminal fragment of the selectable marker protein and (ii) a nucleotide sequence encoding a third molecule of interest, wherein the N-terminal fragment and the C-terminal fragment of the first intein catalyze joining of N-terminal fragment of the selectable marker protein to the central fragment of the selectable markerprotein, and the N-terminal fragment and the C-terminal fragment of the second intein catalyze joining of central fragment of the selectable markerprotein to the C-terminal fragment of the selectable markerprotein, to produce a full-length selectable markerprotein.
218. A transgenic selection method comprising delivering to eukaryotic cells (a) a first vector comprising (i) a nucleotide sequence encoding an N-terminal fragment of a selectable marker protein (e.g., antibiotic resistance protein or fluorescent protein), which is upstream from a nucleotide sequence encoding an N-terminal fragment of a first intein and (ii) a nucleotide sequence encoding a first molecule of interest, (b) a second vector comprising (i) a nucleotide sequence encoding a C-terminal fragment of the first intein, which is upstream from a nucleotide sequence encoding a first central fragment of the selectable marker protein, which is upstream from a nucleotide sequence encoding an N-terminal fragment of a second intein and (ii) a nucleotide sequence encoding a second molecule of interest, (c) a third vector comprising (i) a nucleotide sequence encoding a C-terminal fragment of the second intein, which is upstream from a nucleotide sequence encoding a second central fragment of the selectable marker protein, which is upstream from a nucleotide sequence encoding an N-terminal fragment of a third intein and (ii) a nucleotide sequence encoding a third molecule of interest, and (d) a fourth vector comprising (i) a nucleotide sequence encoding a C-terminal fragment of the third intein, which is upstream from a nucleotide sequence encoding a C-terminal fragment of the selectable marker protein and (ii) a nucleotide sequence encoding a third molecule of interest, wherein the N-terminal fragment and the C-terminal fragment of the first intein catalyze joining of N-terminal fragment of the selectable marker protein to the first central fragment of the selectable marker protein, the N-terminal fragment and the C-terminal fragment of the second intein catalyze joining of first central fragment of the selectable marker protein to the second central fragment of the selectable marker protein, the N-terminal fragment and the C-terminal fragment of the third intein catalyze joining of second central fragment of the selectable marker protein to the C-terminal fragment of the selectable marker protein to produce a full-length selectable marker protein.
219. The method of any one of paragraphs 216-218 further comprising maintaining the eukaryotic cells under conditions that permit introduction of the vectors into the eukaryotic cells to produce transgenic eukaryotic cells.
220. The method of paragraph 219 further comprising selecting the transgenic eukaryotic cells that comprise the full-length selectable marker protein.
221. The method of any one of paragraphs 216-220, wherein the eukaryotic cells are mammalian cells.
222. The method of any one of paragraphs 216-221, wherein the antibiotic resistance protein confers resistance to hygromycin, G418, puromycin, phleomycin D1 or blasticidin.
223. The method of any one of paragraphs 216-222, wherein the intein is a split intein.
224. The method of paragraph 223, wherein the split intein is a natural split intein.
225. The method of paragraph 224, wherein the natural split intein is selected from DnaE inteins.
226. The method of paragraph 225, wherein the DnaE inteins are selected from Synechocystis sp. DnaE (SspDnaE) inteins and Nostoc punctiforme (NpuDnaE) inteins.
227. The method of paragraph 223, wherein the split intein is an engineered split intein.
228. The method of paragraph 2278, wherein the engineered split intein is engineered from DnaB inteins.
229. The method of paragraph 228, wherein the engineered split intein is a SspDnaB S1 intein.
230. The method of paragraph 229, wherein the engineered split intein is engineered from GyrB inteins.
231. The method of paragraph 230, wherein the engineered split intein is a SspGyrB S11 intein.
232. The method of any one of paragraphs 216-231, wherein the molecules are selected from proteins.
233. The method of any one of paragraphs 216-231, wherein the molecules are selected from non-coding ribonucleic acids (RNAs).
234. The method of paragraph 233, wherein the non-coding RNAs are selected from microRNAs (miRNAs), antisense RNAs, short-interfering RNAs (siRNAs), and short-hairpin RNAs (shRNAs).
235. The method of any one of paragraphs 216-234, wherein the vectors are selected from plasmid vectors and viral vectors.

### EXAMPLES

The present disclosure is further illustrated by the following Examples. These Examples are provided to aid in the understanding of the disclosure, and should not be construed as a limitation thereof.

### Example 1. Antiobiotic Resistance Markers

Selectable markers are often used in genetic engineering to isolate cells with desired genotypes [1]. However, there are a limited number of well-characterized antibiotic resistance genes for use in eukaryotic cells and a limited number of fluorescent proteins whose spectra can be unambiguously differentiated by equipment in ordinary laboratories. Researchers often run into the problem of not having enough choices of selectable markers if they are to incorporate multiple transgenes into a cell. On the other hand, selection with multiple antibiotics at the same time is often harsh to cells. “Selectable marker recycling” may provide a work-around, however, requiring multiple rounds of transgenesis, selection and removal of selection markers [2]. To allow multiple transgenes to be selected by one selection scheme at the same time, we have created split antibiotics resistance and fluorescent protein genes wherein a gene encoding an antibiotic resistance or fluorescent protein is split into two or more segments fused to inteins (“markertrons”) that can be rejoined by protein trans-splicing [3] (FIG. 1A). Each markertron is inserted onto a transgenic vector carrying a specific transgene. Delivery of transgenic vectors containing a set of markertrons yield cells harboring a subset or a complete set of the marketrons. Only cells containing a complete set of markertrons produce a fully reconstituted marker protein via protein splicing and thus passes through selection while cells with partial sets of markertrons are eliminated, achieving co-selection of cells containing all intended transgenes.

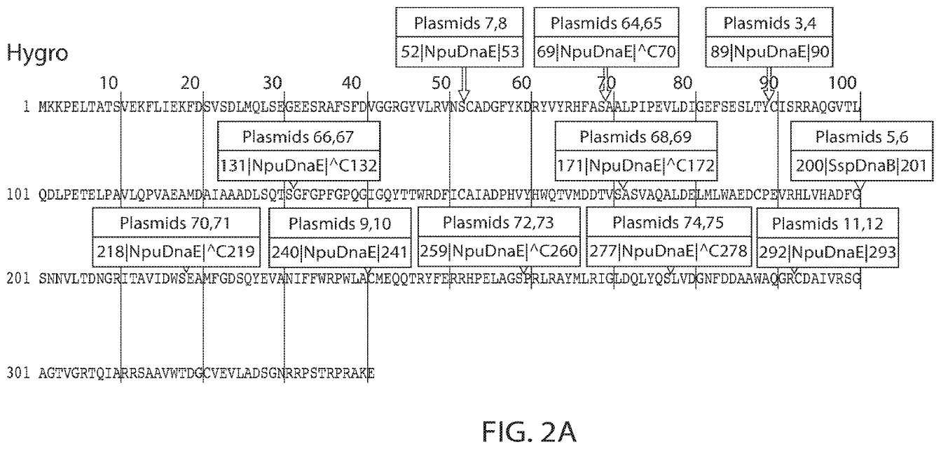

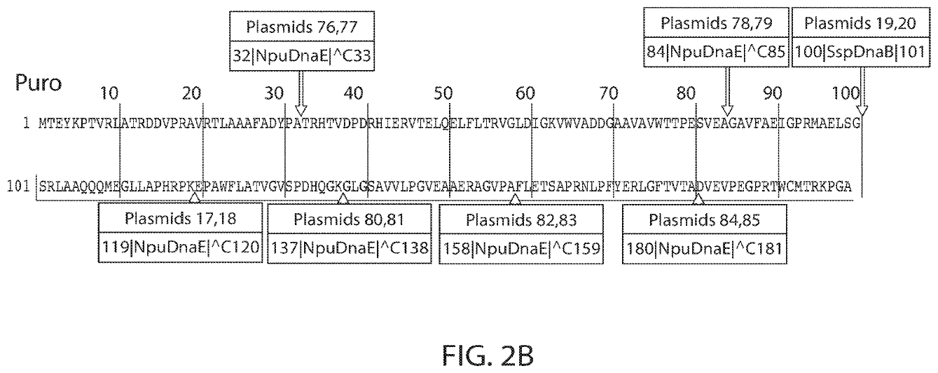

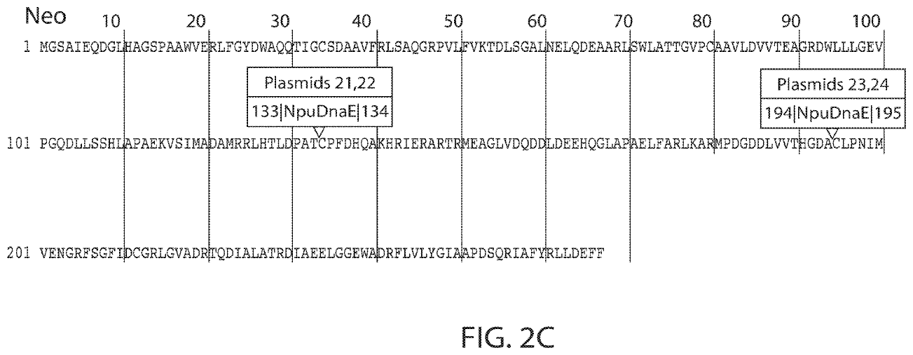

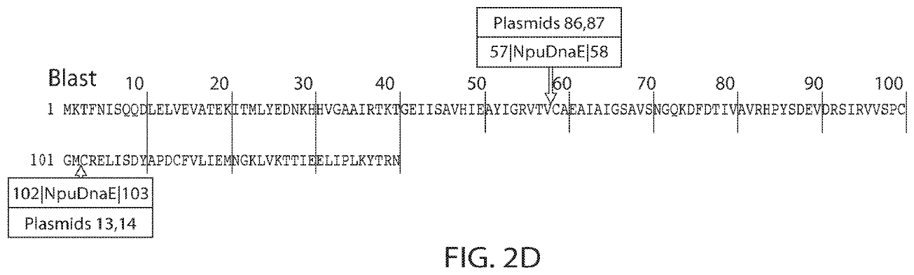

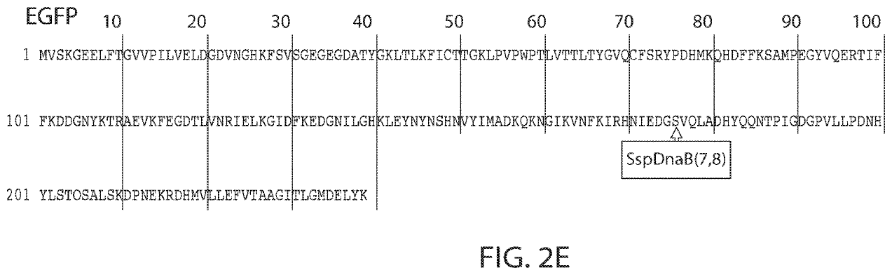

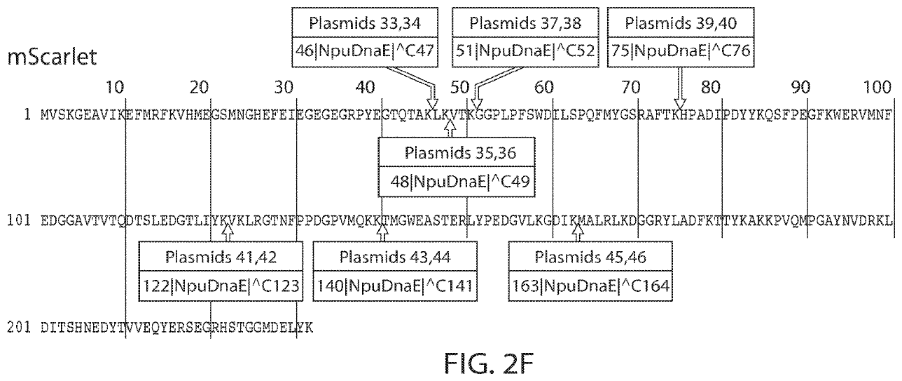

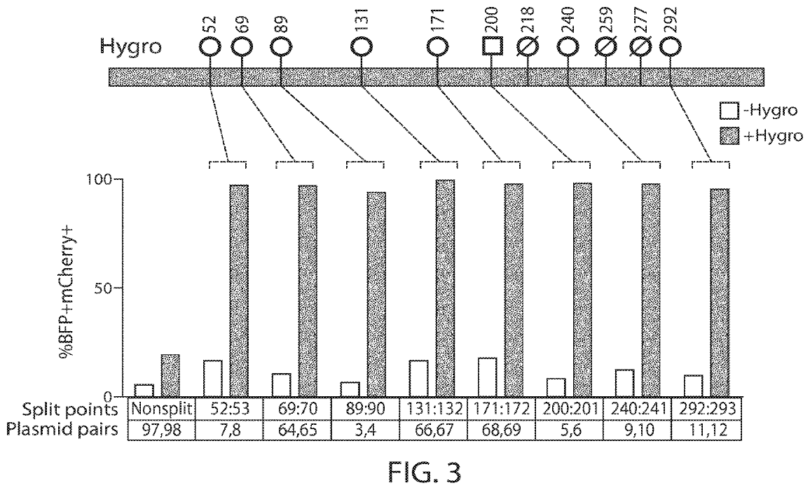

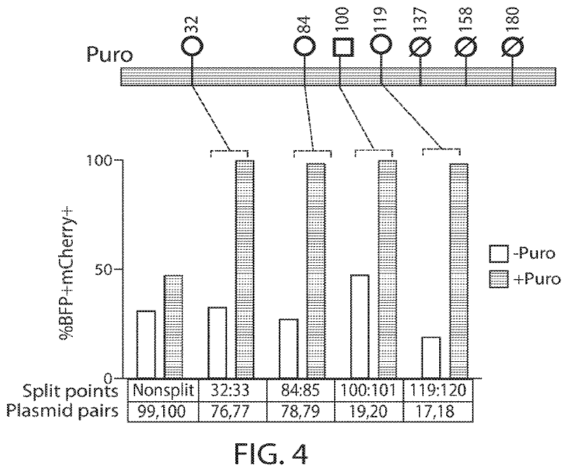

We started out with engineering 2-markertron intein-split resistance (Intres) genes for double transgenesis. Since flanking residues and local protein folding can affect efficiency of intein-mediated trans-splicing, we set out to identify split points in each of the four commonly used antibiotic resistance genes compatible with two well-characterized split inteins derived from NpuDnaE [4, 5] and SspDnaB [6]. To facilitate assessment of the effectiveness of double transgenic selection, we cloned markertrons onto lentiviral vectors expressing TagBFP2 or mCherry fluorescent proteins as test transgenes (FIG. 1B). Viral preparations were transduced into U2OS cells which were then split into replicate plates with non-selective or selective media. Following appropriate passages for antibiotics selection, the two cell cultures were analyzed by flow cytometry. For hygromycin (Hygro) resistance gene, one “native” SspDnaB split point (G200:S201) with flanking residues “GS” and one “native” NpuDnaE split point (Y89:C90) with “YC” residues were tested. Both enabled successful selection when both N- and C-markertrons were transduced yielding >99% BFP+ mCherry+ double transgenic cells in selected cultures compared to <10% double-positive cells in non-selected culture (FIG. 3; Plasmid pairs 3,4 and 5,6). Cells transduced with either of the two markertrons did not survive hygromycin selection. In contrast, double transgenesis with conventional full-length non-split hygromycin vectors only allow for ˜20% enrichment of BFP+ mCherry+ cells (Plasmid pairs 97,98). We screened three addition potential split points (52S:53C), (240A:241C), and (292R:293C) for NpuDnaE with the obligatory cysteine residue on the C-extein junction and a residue on the N-extein junction that supported substantial trans-splicing activities in a previous report 7. We also incorporated six additional NpuDnaE split points by inserting an “artificial” cysteine on the C-extein junction to support splicing at ectopic sites yielding additional split points. In total, eight out of eleven split points tested supported hygromycin selection (FIG. 3). Similarly, for puromycin (Puro) (FIG. 4), neomycin (Neo) (FIG. 5) and blasticidin (Blast) (FIG. 6) resistance genes, we identified four, two, and one functional Intres pair(s), respectively. In all of these cases, cells transduced with either markertrons did not survive selection, while cells transduced with both yielded >95% double transgenic cells in selective cultures compared to <50% in non-selective cultures with the exception of Blasticidin(102) Intres, achieving lower but still significant enrichment of 91% double transgenic cells (FIGS. 3-6). Details of the split points of Intres genes and plasmids are presented in FIGS. 2A-2D and Table 1.

TABLE 1
Plasmids

| Plasmid #* | Plasmid Name | Markertron |
|---|---|---|
| 3 | pLX-Hygro(1-89)-NpuDnaE(N)-IRES-TagBFP2 | Hygro(1-89)-NpuDnaE(N) |
| 4 | pLX-NpuDnaE(C)-Hygro(90-341)-IRES-mCherry | NpuDnaE(C)-Hygro(90-341) |
| 5 | pLX-Hygro(1-200)-SspDnaB(N)-IRES-TagBFP2 | Hygro(1-200)-SspDnaB(N) |
| 6 | pLX-SspDnaB(C)-Hygro(201-341)-IRES-mCherry | SspDnaB(C)-Hygro(201-341) |
| 7 | pLX-Hygro(1-52)-NpuDnaE(N)-IRES-TagBFP2 | Hygro(1-52)-NpuDnaE(N) |
| 8 | pLX-NpuDnaE(C)-Hygro(53-341)-IRES-mCherry | NpuDnaE(C)-Hygro(53-341) |
| 9 | pLX-Hygro(1-240)-NpuDnaE(N)-IRES-TagBFP2 | Hygro(1-240)-NpuDnaE(N) |
| 10 | pLX-NpuDnaE(C)-Hygro(241-341)-IRES-mCherry | NpuDnaE(C)-Hygro(241-341) |
| 11 | pLX-Hygro(1-292)-NpuDnaE(N)-IRES-TagBFP2 | Hygro(1-292)-NpuDnaE(N) |
| 12 | pLX-NpuDnaE(C)-Hygro(293-341)-IRES-mCherry | NpuDnaE(C)-Hygro(293-341) |
| 13 | pLX-Blast(1-102)-NpuDnaE(N)-IRES-TagBFP2 | Blast(1-102)-NpuDnaE(N) |
| 14 | pLX-NpuDnaE(C)-Blast(103-140)-IRES-mCherry | NpuDnaE(C)-Blast(103-140) |
| 17 | pLX-Puro(1-119)-NpuDnaE(N)-IRES-TagBFP2 | Puro(1-119)-NpuDnaE(N) |
| 18 | pLX-NpuDnaE(C)-Puro(insCys; 120-199)-IRES-mCherry | NpuDnaE(C)-Puro(insCys; 120-199) |
| 19 | pLX-Puro(1-100)-SspDnaB(N-S0)-IRES-TagBFP2 | Puro(1-100)-SspDnaB(N-S0) |
| 20 | pLX-SspDnaB(C-S0)-Puro(101-199)-IRES-mCherry | SspDnaB(C-S0)-Puro(101-199) |
| 21 | pLX-Neo(1-133)-NpuDnaE(N)-IRES-TagBFP2 | Neo(1-133)-NpuDnaE(N) |
| 22 | pLX-NpuDnaE(C)-Neo(134-267)-IRES-mCherry | NpuDnaE(C)-Neo(134-267) |
| 23 | pLX-Neo(1-194)-NpuDnaE(N)-IRES-TagBFP2 | Neo(1-194)-NpuDnaE(N) |
| 24 | pLX-NpuDnaE(C)-Neo(195-267)-IRES-mCherry | NpuDnaE(C)-Neo(195-267) |
| 25 | pLX-NpuDnaE(C)_Hygro(53-89)-NpuDnaE(N)-IRES-GFP | NpuDnaE(C)_Hygro(53-89)-NpuDnaE(N) |
| 26 | pLX-NpuDnaE(C)_Hygro(53-239)-NpuDnaE(N)-IRES-GFP | NpuDnaE(C)_Hygro(53-239)-NpuDnaE(N) |
| 27 | pCR8-BsaI−>ccdbCam<−BsaI-NpuDnaE(N)-MD1-68-15 | |
| 28 | pCR8-NpuDnaE(C)_BsaI−>ccdbCam<−BsaI-MD1-68-18 | |
| 29 | pCR8-BsaI−>ccdbCam<−BsaI-SspDnaE(N)-MD1-68-12 | |
| 30 | pCR8-SspDnaE(C)_BsaI−>ccdbCam<−BsaI-MD1-68-13 | |
| 31 | pCR8-BsaI−>ccdbCam<−BsaI-SspDnaB(N-S0)-25-135-18 | |
| 32 | pCR8-SspDnaB(C-S0)_BsaI−>ccdbCam<−BsaI-25-155-41 | |
| 33 | pLX-mScarlet(1-46)-NpuDnaE(N)_LZA-IRES-TagBFP2 | mScarlet(1-46)-NpuDnaE(N)_LZA |
| 34 | pLX-LZB_NpuDnaE(C)-mScarlet(insCys; 47-232)-IRES- | LZB_NpuDnaE(C)-mScarlet(insCys; 47-232) |
| | TagBFP2 | |
| 35 | pLX-mScarlet(1-48)-NpuDnaE(N)_LZA-IRES-TagBFP2 | mScarlet(1-48)-NpuDnaE(N)_LZA |
| 36 | pLX-LZB_NpuDnaE(C)-mScartet(insCys; 49-232)-IRES-GFP | LZB_NpuDnaE(C)-mScarlet(insCys; 49-232) |
| 37 | pLX-mScarlet(1-51)-NpuDnaE(N)_LZA-IRES-TagBFP2 | mScarlet(1-51)-NpuDnaE(N)_LZA |
| 38 | pLX-LZB_NpuDnaE(C)-mScarlet(insCys; 52-232)-IRES-GFP | LZB_NpuDnaE(C)-mScarlet(insCys; 52-232) |
| 39 | pLX-mScarlet(1-75)-NpuDnaE(N)_LZA-IRES-TagBFP2 | mScarlet(1-75)-NpuDnaE(N)_LZA |
| 40 | pLX-LZB_NpuDnaE(C)-mScarlet(insCys; 76-232)-IRES-GFP | LZB_NpuDnaE(C)-mScarletflnsCys; 76-232) |
| 41 | pLX-mScarlet(1-122)-NpuDnaE(N)_LZA-IRES-TagBFP2 | mScarlet(1-122)-NpuDnaE(N)_LZA |
| 42 | pLX-LZB_NpuDnaE(C)-mScarlet(insCys; 123-232)-IRES-GFP | LZB_NpuDnaE(C)-mScarlet(insCys; 123- |
| | | 232) |
| 43 | pLX-mScarlet(1-140)-NpuDnaE(N)_LZA-IRES-TagBFP2 | mScarlet(1-140)-NpuDnaE(N)_LZA |
| 44 | pLX-LZB_NpuDnaE(C)-mScarlet(insCys; 141-232)-IRES-GFP | LZB_NpuDnaE(C)-mScarlet(insCys; 141- |
| | | 232) |
| 45 | pLX-mScarlet(1-163)-NpuDnaE(N)_LZA-IRES-TagBFP2 | mScarlet(1-163)-NpuDnaE(N)_LZA |
| 46 | pLX-LZB_NpuDnaE(C)-mScarlet(insCys; 164-232)-IRES-GFP | LZB_NpuDnaE(C)-mScarlet(insCys; 164- |
| | | 232) |
| 47 | pCR8-TagBFP2 | TagBFP2 |
| 48 | pCR8-mCherry | mCherry |
| 49 | pLX-DEST-IRES-Hygro(1-89)-NpuDnaE(N) | Hygro(1-89)-NpuDnaE(N) |
| 50 | pLX-DEST-IRES-NpuDnaE(C)-Hygro(90-341) | NpuDnaE(C)-Hygro(90-341) |
| 51 | pLX-[TagBFP2]-IRES-Hygro(1-89)-NpuDnaE(N) | Hygro(1-89)-NpuDnaE(N) |
| 52 | pLX-[mCherry]-IRES-NpuDnaE(C)-Hygro(90-341) | NpuDnaE(C)-Hygro(90-341) |
| 53 | pLX-DEST-IRES-Puro(1-119)-NpuDnaE(N) | Puro(1-119)-NpuDnaE(N) |
| 54 | pLX-DEST-IRES-NpuDnaE(C)-Puro(120-199) | NpuDnaE(C)-Puro(insCys; 120-199) |
| 55 | pLX-[TagBFP2]-IRES-Puro(1-119)-NpuDnaE(N) | Puro(1-119)-NpuDnaE(N) |
| 56 | pLX-[mCherry]-IRES-NpuDnaE(C)-Puro(120-199) | NpuDnaE(C)-Puro(insCys; 120-199) |
| 57 | pLX-DEST-IRES-Neo(1-194)-NpuDnaE(N) | Neo(1-194)-NpuDnaE(N) |
| 58 | pLX-DEST-IRES-NpuDnaE(C)-Neo(195-267) | NpuDnaE(C)-Neo(195-267) |
| 59 | pLX-[TagBFP2]-IRES-Neo(1-194)-NpuDnaE(N) | Neo(1-194)-NpuDnaE(N) |
| 60 | pLX-[mCherry]-IRES-NpuDnaE(C)-Neo(195-267) | NpuDnaE(C)-Neo(195-267) |
| 64 | pLX-Hygro(1-69)-NpuDnaE(N)-IRES-TagBFP2 | Hygro(1-69)-NpuDnaE(N) |
| 65 | pLX-NpuDnaE(C)-Hygro(\hat{ }C; 70-341)-IRES-mCherry | NpuDnaE(C)-Hygro(\hat{ }C; 70-341) |
| 66 | pLX-Hygro(1-131)-NpuDnaE(N)-IRES-TagBFP2 | Hygro(1-131)-NpuDnaE(N) |
| 67 | pLX-NpuDnaE(C)-Hygro(\hat{ }C; 132-341)-IRES-mCheny | NpuDnaE(C)-Hygro(\hat{ }C; 132-341) |
| 68 | pLX-Hygro(1-171)-NpuDnaE(N)-IRES-TagBFP2 | Hygro(1-171)-NpuDnaE(N) |
| 69 | pLX-NpuDnaE(C)-Hygro(\hat{ }C; 172-341)-IRES-mCherry | NpuDnaE(C)-Hygro(\hat{ }C; 172-341) |
| 70 | pLX-Hygro(1-218)-NpuDnaE(N)-IRES-TagBFP2 | Hygro(1-218)-NpuDnaE(N) |
| 71 | pLX-NpuDnaE(C)-Hygro(\hat{ }C; 219-341)-IRES-mCherry | NpuDnaE(C)-Hygro(\hat{ }C; 219-341) |
| 72 | pLX-Hygro(1-259)-NpuDnaE(N)-IRES-TagBFP2 | hygro(1-259)-NpuDnaE(N) |
| 73 | pLX-NpuDnaE(C)-Hygro(\hat{ }C; 260-341)-IRES-mCherry | NpuDnaE(C)-Hygro(\hat{ }C; 260-341) |
| 74 | pLX-Hygro(1-277)-NpuDnaE(N)-IRES-TagBFP2 | Hygro(1-277)-NpuDnaE(N) |
| 75 | pLX-NpuDnaE(C)-Hygro(\hat{ }C; 278-341)-IRES-mCherry | NpuDnaE(C)-Hygro(\hat{ }C; 278-341) |
| 76 | pLX-Puro(1-32)-NpuDnaE(N)-IRES-TagBFP2 | Puro(1-32)-NpuDnaE(N) |
| 77 | pLX-NpuDnaE(C)-Puro(\hat{ }C; 33-199)-IRES-mCherry | NpuDnaE(C)-Puro(\hat{ }C; 33-199) |
| 78 | pLX-Puro(1-84)-NpuDnaE(N)-IRES-TagBFP2 | Puro(1-84)-NpuDnaE(N) |
| 79 | pLX-NpuDnaE(C)-Puro(\hat{ }C; 85-199)-IRES-mCherry | NpuDnaE(C)-Puro(\hat{ }C; 85-199) |
| 80 | pLX-Puro(1-137)-NpuDnaE(N)-IRES-TagBFP2 | Puro(1-137)-NpuDnaE(N) |
| 81 | pLX-NpuDnaE(C)-Puro(\hat{ }C; 138-199)-IRES-mCherry | NpuDnaE(C)-Puro(\hat{ }C; 138-199) |
| 82 | pLX-Puro(1-158)-NpuDnaE(N)-IRES-TagBFP2 | Puro(1-158)-NpuDnaE(N) |
| 83 | pLX-NpuDnaE(C)-Puro(\hat{ }C; 159-199)-IRES-mCherry | NpuDnaE(C)-Puro(\hat{ }C; 159-199) |
| 84 | pLX-Puro(1-180)-NpuDnaE(N)-IRES-TagBFP2 | Puro(1-180)-NpuDnaE(N) |
| 85 | pLX-NpuDnaE(C)-Puro(\hat{ }C; 181-199)-IRES-mCherry | NpuDnaE(C)-Puro(\hat{ }C; 181-199) |
| 86 | pLX-Blast(1-58)-NpuDnaE(N)-IRES-TagBFP2 | Blast(1-58)-NpuDnaE(N) |
| 87 | pLX-NpuDnaE(C)-Blast(59-140)-IRES-mCherry | NpuDnaE(C)-Blast(59-140) |
| 88 | pLX-NpuDnaE(C)-HygroBA-SspDnaB(N-S0)-IRES-EGFP | NpuDnaE(C)-Hygro(53-200)-SspDnaB(N- |
| | | S0) |
| 89 | pLX-SspDnaB(C-S0)-Hygro(201-341)-IRES-mCherry | SspDnaB(C-S0)-Hygro(201-341) |
| 90 | pLX-NpuDnaE(C)-Hygro(90-200)-SspDnaB(N-S0)-IRES- | NpuDnaE(C)-Hygro(90-200)-SspDnaB(N- |
| | EGFP | S0) |
| 91 | pLX-Hygro(1-200)-SspDnaB(N-S0)-IRES-TagBFP2 | Hygro(1-200)-SspDnaB(N-S0) |
| 92 | pLX-SspDnaB(C-S0)-Hygro(201-240)-NpuDnaE(N)-IRES- | SspDnaB(C-S0)-Hygro(201-240)- |
| | EGFP | NpuDnaE(N) |
| 93 | pLX-SspDnaB(C-S0)-Hygro(201-292)-NpuDnaE(N)-IRES- | SspDnaB(C-S0)-Hygro(201-292)- |
| | EGFP | NpuDnaE(N) |
| 94 | pLX-DEST-IRES-TagBFP2 | |
| 95 | pLX-DEST-IRES-EGFP | |
| 96 | pLX-DEST-IRES-mCherry | |
| 97 | pLX-Hygro-IRES-TagBFP2 | Non-split Hygro |
| 98 | pLX-Hygro-IRES-mCherry | Non-split Hygro |
| 99 | pLX-Puro-IRES-TagBFP2 | Non-split Puro |
| 100 | pLX-Puro-IRES-mCherry | Non-split Puro |
| 101 | pLX-Hygro-IRES-EGFP | Non-split Hygro |
| 102 | pLX-NLS_GFP-IRES-Hygro | Non-split Hygro |
| 103 | pLX-LifeAct_mCherry-IRES-Hygro | Non-split Hygro |
| 104 | pLX-NLS_GFP-IRES-Hygro(1-89)-NpuDnaE(N) | Hygro(1-89)-NpuDnaE(N) |
| 105 | pLX-LifeAct_mScarlet-IRES- NpuDnaE(C)-Hygro(90-341) | NpuDnaE(C)-Hygro(90-341) |
| 106 | pX330-AAVS1 | |
| 107 | pAAVS1-Nst-EF1aHygro2ArtTA3(−)_TetO-Blast-P2A-EGFP | Non-split Blast |
| 108 | pAAVS1-Nst-EF1aHygro2ArtTA3(−)_TetO-Blast-P2A-mScarlet | Non-split Blast |
| 109 | pAAVS1-Nst-EF1aHygro2ArtTA3(−)_TetO-Blast(1- | Blast(1-102)-NpuDnaE(N) |
| | 102)_NpuDnaE(N)-P2A-EGFP | |
| 110 | pAAVS1-Nst-EF1aHygro2ArtTA3(−)_TetO- | NpuDnaE(C)-Blast(103-140) |
| | NpuDnaE(C)_Blast(103-140)-P2A-mScarlet | |
| 111 | pAAVS1-Nst-EF1aBlast2ArtTA3(−)_TetO-Hygro-P2A-NTR- | Non-split Hygro |
| | E2A-EGFP | |
| 112 | pAAVS1-Nst-EF1aBlast2ArtTA3(−)_TetO-Hygro-P2A-NTR- | Non-split Hygro |
| | E2A-mCherry | |
| 113 | pAAVS1-Nst-EF1aBlast2ArtTA3(−)_TetO- Hygro(1-89)- | Hygro(1-89)-NpuDnaE(N) |
| | NpuDnaE(N)-P2A-NTR-E2A-EGFP | |
| 114 | pAAVS1-Nst-EF1aBlast2ArtTA3(−)_TetO- NpuDnaE(C)- | NpuDnaE(C)-Hygro(90-341) |
| | Hygro(90-341)-P2A-NTR-E2A-mCherry | |
| 115 | pLX-Hygro(1-89)_NpuDnaE(N)_LZA-IRES-TagBFP2 | Hygro(1-89)-NpuDnaE(N)-LZA |
| 116 | pLX-LZB_NpuDnaGEP(C)_Hygro(90-200)_SspDnaB(N-S0)- | LZB-NpuDnaGEP(C)-Hygro(90-200)- |
| | IRES-GFP | SspDnaB(N-S0) |
| 117 | pLX-SspDnaB(C-S0)_Hygro(201-240)_NpuDnaE(N)_LZA- | SspDnaB(C-S0)-Hygro(201-240)- |
| | IRES-GFP | NpuDnaE(N)-LZA |
| 118 | pLX-LZB_NpuDnaGEP(C)_Hygro(241-341 )-IRES-mCherry | LZB-NpuDnaGEP(C)-Hygro(241-341) |

### Example 2. Gateway-Compatible Lentiviral Vectors

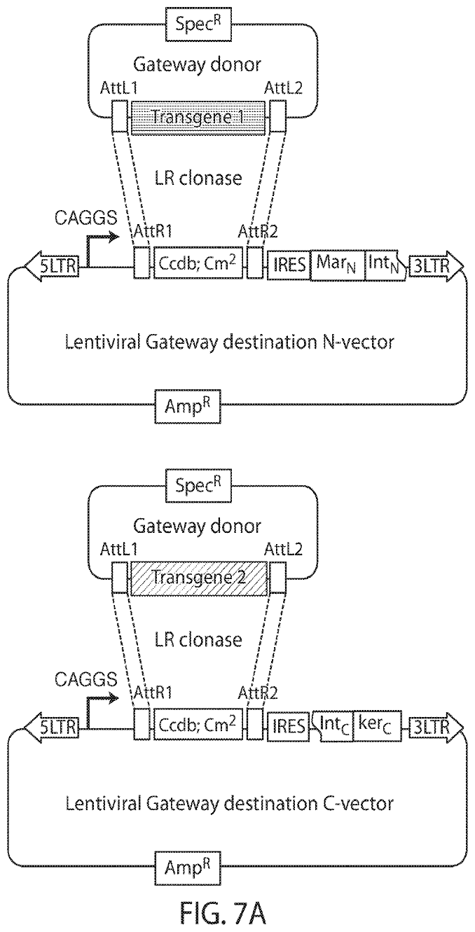

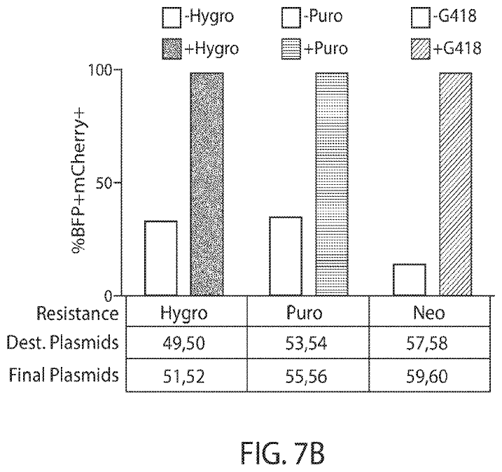

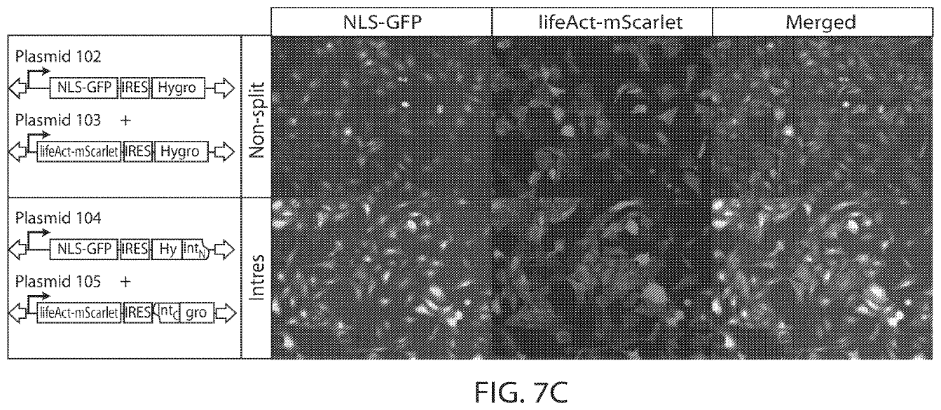

To facilitate adoption of Intres markers, we created Gateway-compatible lentiviral vectors for convenient restriction-ligation-independent LR clonase recombination of transgenes 8 (FIG. 7A). We tested the functionality of these vectors by recombining TagBPF2 and mCherry, respectively to the N- and C-Intres vectors and found robust selection of double transgenic cells (FIG. 7B). One potential utility of Intres vectors is to install different fluorescent markers in cells to label different cellular compartments. To explore such utility, we cloned in NLS-GFP and LifeAct-mScarlet 9, which label nucleus and F-actin, respectively, by Gateway recombination to conventional full length (FL) non-split hygromycin selectable vectors or 2-markertron hygromycin Intres vectors and transduced cells with either sets of plasmids, followed by antibiotic selection (FIG. 7C). The sample transduced with non-split selectable plasmids contained both singly and doubly labelled cells, while cell transduced with Intres plasmids were all doubly labelled (FIG. 7C).

### Example 3. Fluorescent Markers

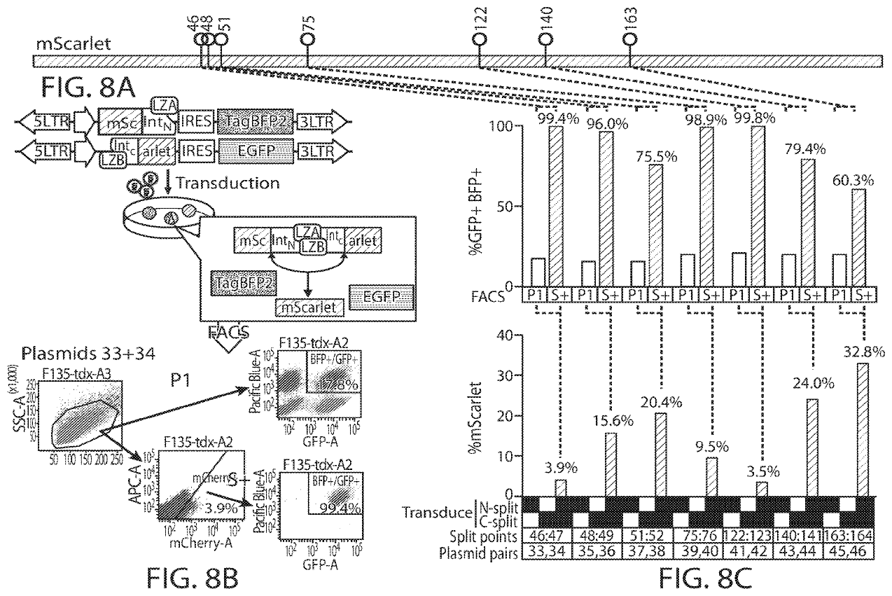

To test whether split fluorescent markers can be used for transgene selection, we screened for NpuDnaE split points for mScarlet fluorescent protein (FIG. 8A) and identified four split points allowing for >96% enrichment of double transgenic cells and three other split points enabling >60% enrichment of double transgenic cells in mScarlet-gated population, compared to <20% double transgenic cells in non-gated population (FIG. 8B).

### Example 4. Higher Degree Split Markers

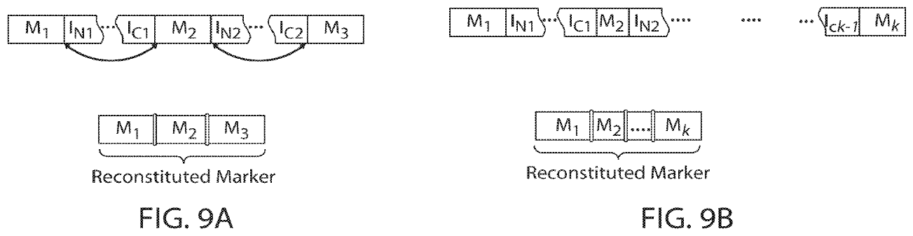

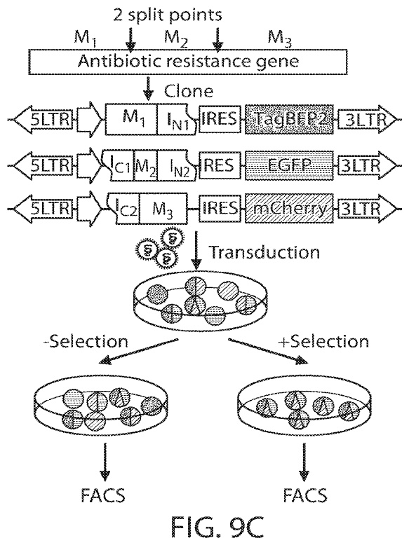

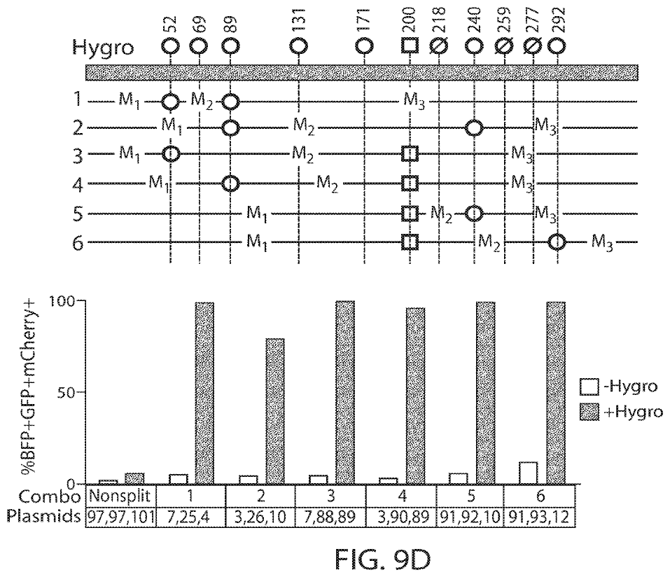

With the split points identified for 2-markertron Intres genes, we set out to engineer higher degree split markers. We tested combinations of splits points to partition a marker gene into three or more markertrons to allow for co-selection of more than two “unlinked” transgenes with one antibiotics (FIGS. 9A-9B). To identify pairs of split points that would allow such “Intres chain”, we cloned 3-split markertrons into three lentiviral vectors each carrying one of three fluorescent transgenes TagBFP2, EGFP, or mCherry, that will allow us to assess effectiveness of selection by flow cytometry (FIG. 9C). Since hygromycin resistance gene is the longest and provides the most split points for testing, we focused on engineering 3-markertron hygromycin Intres. We tested two 3-markertron hygromycin Intres using two intervening NpuDnaE inteins, two using NpuDnaE for the first intein and SspDnaB for the second intein, as well as two using SspDnaB for the first intein and NpuDnaE for the second intein (FIG. 9D). Five of these six 3-markertron hygromycin Intres enabled >97% and with the remaining one enabling 80% triple transgenic selection in hygromycin-selected cultures compared to <15% triple transgenic cells in non-selected cultures. Samples with leave-one-out transduction did not yield any viable cells after hygromycin selection while cells transduced with non-split hygromycin vectors yielded only 7% triple transgenic cells after selection.

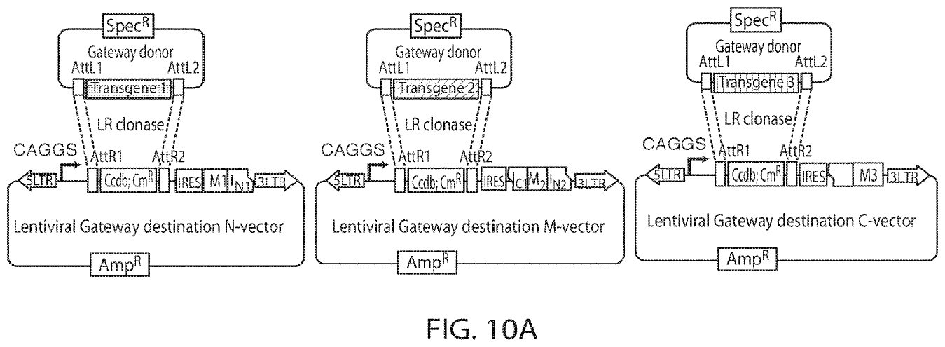

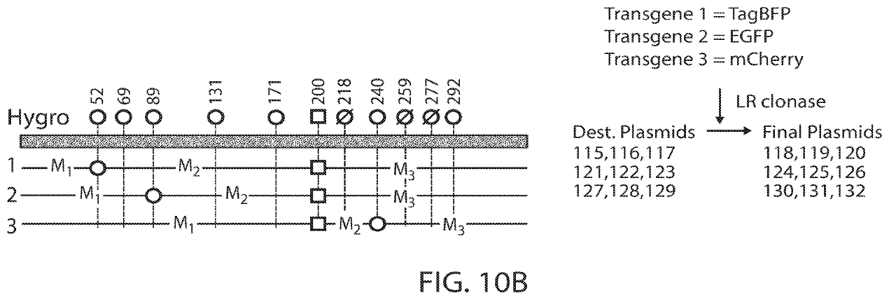

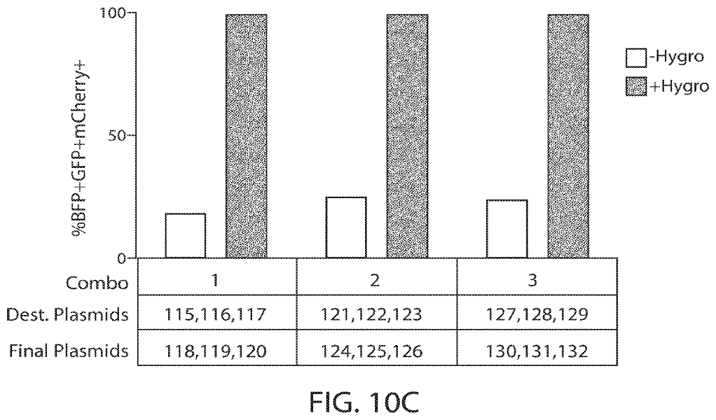

To facilitate the use of 3-markertron Intres, we created Gateway compatible lentiviral vectors with these markers (FIG. 10A). Three sets of these vectors were each tested by recombining TagBFP (as transgene 1), EGFP (as transgene 2) and mCherry (as transgene 3) into the N-, M-, and C-Intres Gateway destination vectors and used to transduce U2OS cells, which were then split and cultured in hygromycin selection or non-selective media (FIG. 10B). Two weeks after selection, cells were analyzed by flow cytometry. All three sets of 3-markertron hygromycin Intres plasmids support triple transgenic cell selection of >99% compared to <25% in the non-selected cultures (FIG. 10C).

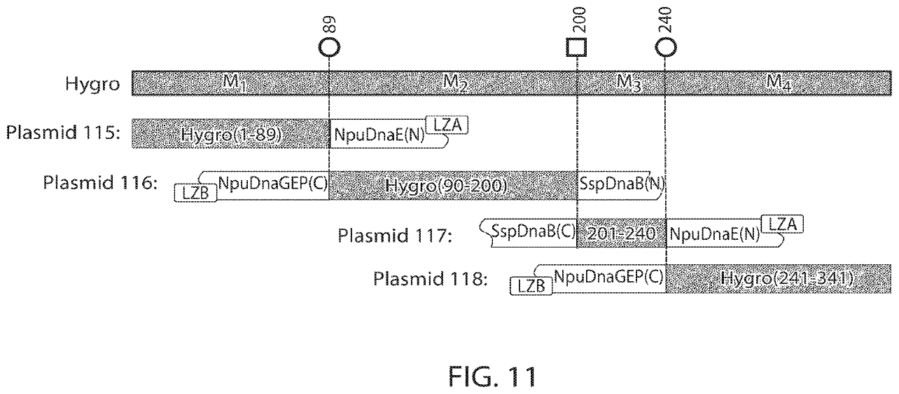

We further tested the feasibility of 4-markertron hygromycin Intres genes (FIG. 11). Here, we used an enhanced variant of NpuDnaE intein known as NpuDnaGEP 10 fused with leucine zipper motifs 11 in combination with the SspDnaB intein. While transduction of all four plasmids containing constituent markertrons produced cells that survived hygromycin selection, leave-one-out transduction did not yield any survival (Table 2).

TABLE 2
Survival of cells transduced with (“+”) or without (“−”) lentiviruses
prepared from the indicated plasmids.

| | Plasmid | Plasmid | Plasmid | Plasmid | |
|---|---|---|---|---|---|
| | 115 | 116 | 117 | 118 | Survival |
| Sample 1 | + | + | + | + | Yes |
| Sample 2 | − | + | + | + | No |
| Sample 3 | + | − | + | + | No |
| Sample 4 | + | + | − | + | No |
| Sample 5 | + | + | + | − | No |

### Example 5. Biallelic Knock-In at the AAVS1 Locus

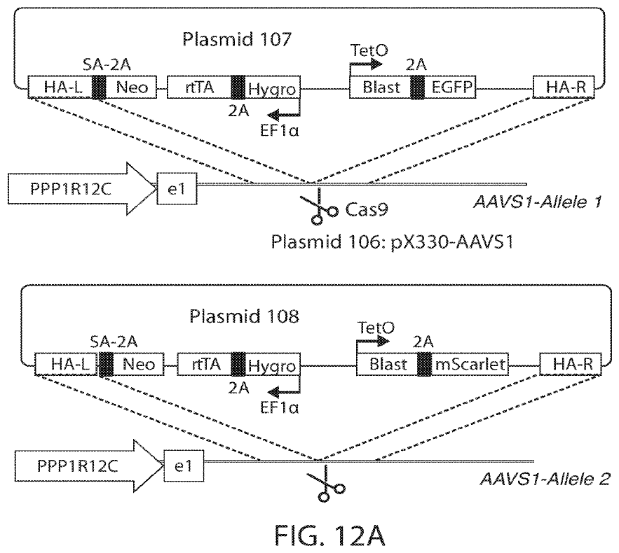

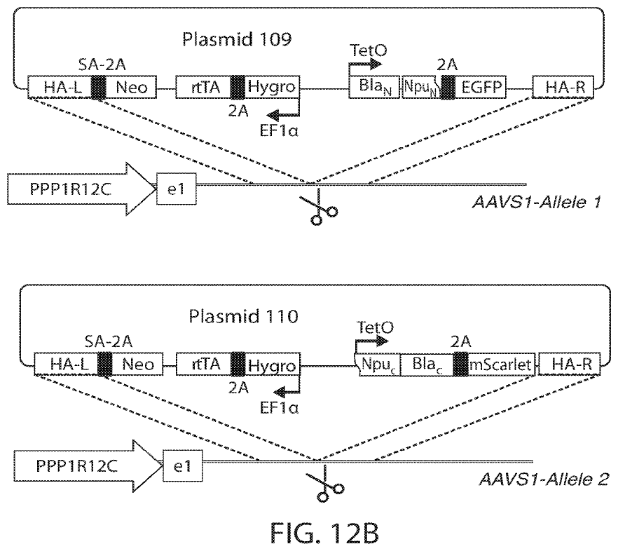

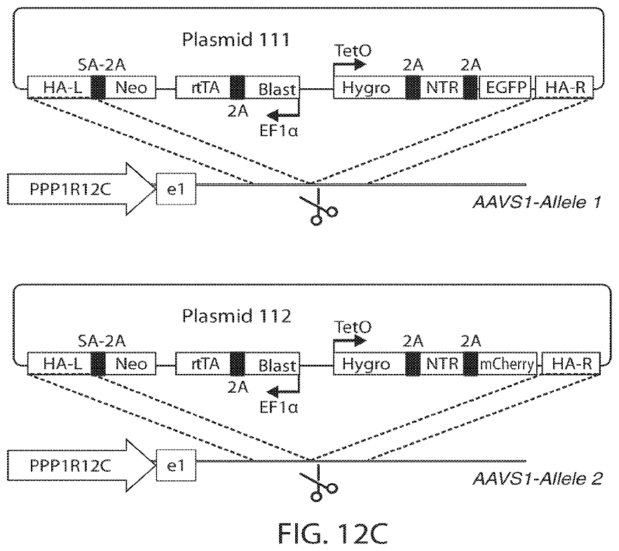

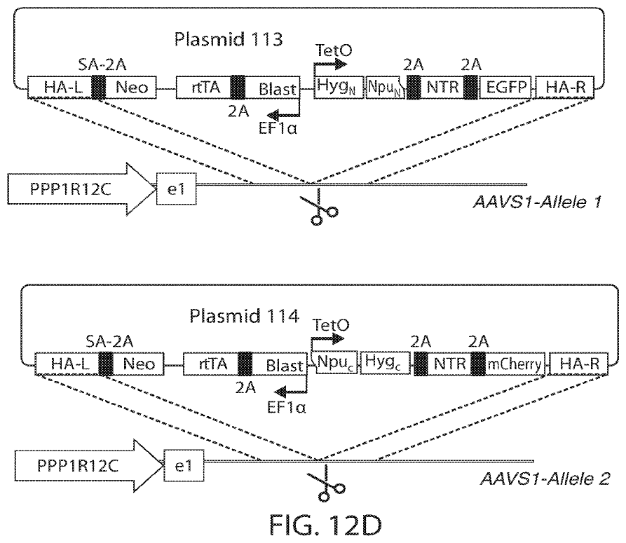

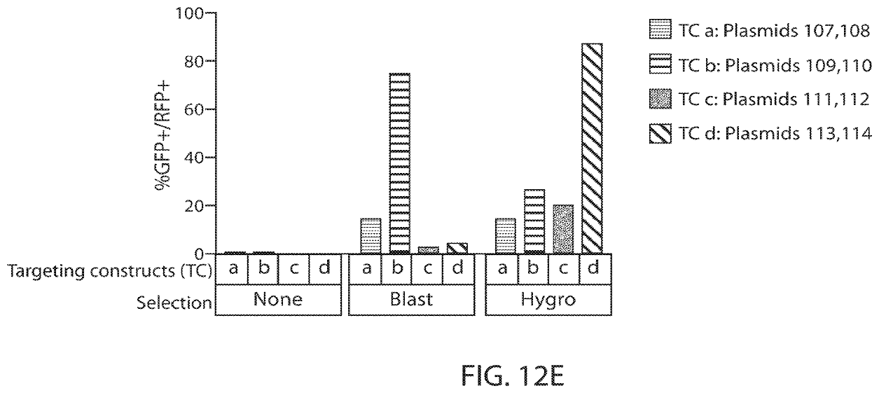

CRISPR/Cas has recently emerged as a powerful technology for genome engineering and editing. Although gene knockout based on NHEJ-mediated insertions/deletions (indels) occur at high frequency, precise editing and knock-in based on homology directed repair (HDR) using exogenous repair templates (a.k.a targeting constructs) are inefficient. We tested whether split selectable markers can be used to enrich for cells with biallelic knock-in at the AAVS1 locus. We constructed targeting constructs with homology arms flanking the target site, and splice acceptor-2A peptide to trap the markertrons within intron one of the host gene PPP1R12C. However, we did not obtain any live cells after CRISPR/Cas knock-in experiments using these targeting constructs and two weeks of antibiotic selection (data not shown). We suspected that the endogenous promoter of the host gene PPP1R12C might not drive sufficient expression of markertrons to reconstitute enough antibiotic resistance protein to counteract actions of the antibiotics. We thus tested an alternative strategy to express Intres markertrons by TetO promoter whose activity can be titrated by doxycycline (dox) concentration. To allow comparison of Intres-mediated biallelic selection versus full-length (FL) non-split selectable markers, we implemented several different targeting construct designs. First, we drive expression of a full-length (FL) resistance gene (e.g., Hygro) together with rtTA under a constitutive EF1a promoter and a separate test Intres (e.g., Blast Intres) under a dox-inducible TetO promoter (FIG. 12B, Plasmids 109 and 110). This allow comparison of full length and split selectable markers within the same constructs. To allow fair comparison of full length versus split markers driven by the same TetO promoter, we constructed two similar plasmids 107 and 108 (cf. Plasmids 109 and 110), wherein full-length antibiotic resistance gene (Blast) is placed downstream of the TetO promoter (FIG. 12A). To enable single-cell quantification of biallelic targeting and to demonstrate the feasibility of incorporating two transgenes into two AAVS1 alleles, we appended EGFP and mScarlet fluorescent genes downstream of the test split or non-split markers via self-cleaving 2A peptide. Similarly, to test Hygro Intres, we swapped the EF1a and TetO-driven markers so that FL Hygro or Hygro Intres were placed downstream of TetO and FL Blast downstream of EF1a (FIGS. 12C-12D; Plasmids 111-114). We co-transfected pX330-AAVS1 (Plasmid 106) containing Cas9 and sgRNA targeting AAVS1, and the different pairs of targeting constructs to HEK293T cells, split into triplicate doxycycline-containing media without antibiotics, with blasticidin, or with hygromycin at the subsequent passages. Two weeks after selection, we analyzed the cultures for biallelic targeting by flow cytometric measurement of GFP and RFP fluorescence (FIG. 12E). As expected, non-selected cultures harbored small fraction (<1%) of biallelic knock-in GFP+/RFP+ cells (FIG. 12E; Selection=None). Selection of antibiotics where corresponding FL antibiotic resistance genes were present on targeting constructs yielded <30% biallelic knock-in cells (FIG. 12E; Blast: TC a,c,d; Hygro: TC a,b,c). In contrast, selection of antibiotics where corresponding Intres are present on the targeting constructs yielded 75% (FIG. 6e; Blast Intres: TC b) and 88% (FIG. 6e; Hygro Intres: TC d) biallelic knock-in cells.

In the Examples above, we have engineered split antibiotic resistance and fluorescent protein genes that can allow selection for two or more “unlinked” transgenes. By inserting unnatural residues at selectable markers, we showed that novel high-efficiency split points can be utilized, expanding the positions available for engineering. We demonstrated that split selectable markers can be incorporated into lentiviral vectors or gene targeting constructs in CRISPR/Cas9 genome editing experiments to enable enrichment of cells with double transgenesis or biallelic knock-ins. By combining two or more splits points, we showed that 3- and 4-split markers can be generated to allow higher degree transgenic selection. Future development of even higher-degree split selectable markers may enable “hyper-engineering” of cells containing tens of transgenes or targeted knock-ins.

### Materials and Methods

Cloning

To generate a test plasmid for each markertron, we first generated a Gateway donor plasmid containing its ORF and then recombine into lentiviral destination vector with TagBFP2 (Plasmid 94: pLX-DEST-IRES-TagBFP2), EGFP (Plasmid 95: pLX-DEST-IRES-EGFP), or mCherry (Plasmid 96: pLX-DEST-IRES-mCherry) reporters, which were derived from pLX302 (addgene.org/25896/) by removing Puromycin resistance gene and inserting IRES-fluorescent genes downstream of the Gateway cassette. The markertron-ORF Gateway donor plasmids were generated either by a nested fusion PCR procedure to combine intein with the coding sequence of fragments of the selectable marker followed by insertion into the pCR8-GW-TOPO plasmid by sequence- and ligation-independent cloning (SLIC) (Li, M. Z. & Elledge, S. J. SLIC: a method for sequence- and ligation-independent cloning. Gene Synthesis: Methods and Protocols, 51-59 (2012)), or PCR-amplifying the relevant fragment of the selectable marker followed by insertion into “scaffold” plasmids (Plasmids 27-32) containing the intein sequences by SLIC. DNA sequences encoding inteins were codon optimized for Homo sapiens, and synthesized as GBlock (IDT), with AC1947 GB encoding NpuDnaE intein, AC1949 GB encoding SspDnaB intein. Selectable marker fragments were amplified from plasmids containing these markers. See Table 1 for plasmids.

Cell Culture

All cells were cultivated in Dulbecco's modified Eagle's medium (DMEM) (Sigma) with 10% fetal bovine serum (FBS)(Lonza), 4% Glutamax (Gibco), 1% Sodium Pyruvate (Gibco) and penicillin-streptomycin (Gibco). Incubator conditions were 37° C. and 5% CO₂.

Virus Production

A viral packaging mix of pLP1, pLP2, and VSV-G were co-transfected with each lentiviral vector into Lenti-X 293T cells (ClonTech), seeded the day before in 6-well plates at a concentration of 1.2×10⁶ cells per well, using Lipofectamine 3000. Media was changed 6 h after transfection then incubated overnight. 28 hour post transfection, the media supernatant containing virus was filtered using 45 uM PES filters then stored at −80° C. until use.

Transduction

The day prior to transduction, target cells (HEK293T, MCF7, U2-OS) were seeded into 12-well plates at a density of 1.5×10⁵ cells per well. Prior to transduction, media was changed to media containing 10 μg/mL polybrene, 1 mL per well. 250 μL of each respective virus (500 μL total for experimental samples with two viruses added) was added to each well and incubated overnight. Media was changed 24 hour post infection. 4 day post infection cells were split into duplicate plates. 5 day post infection media with antibiotic (hygromycin) was added to each respective well of one replicate plate (the other remained under no selection). Antibiotic selection continued for 2 weeks before analysis on FACS.

Fluorescent-Activated Cell Sorting

Cells were trypisinized, suspended in media then analyzed on a LSRFortessa X-20 (BD Bioscience) flow cytometer using FACSDiVa software, version 8, on an HP Z230 workstation. Fifty thousand events were collected each run.

CONSTRUCTS AND SEQUENCES

NpuDnaE(N)
CLSYETEILTVEYGLLPIGKIVEKRIECTVYSVDNNGNIYTQPVAQWHDRGEQEVFEYCLEDGSLIRATKDHKF
MTVDGQMLPIDEIFERELDLMRVDNLPN (SEQ ID NO: 7)
NpuDnaE(C)
IKIATRKYLGKQNVYDIGVERDHNFALKNGFIASN (SEQ ID NO: 8)
SspDnaB(N-S0)
CISGDSLISLASTGKRVSIKDLLDEKDFEIWAINEQTMKLESAKVSRVFCTGKKLVYILKTRLGRTIKATANHRF
LTIDGWKRLDELSLKEHIALPRKLESSSLQL (SEQ ID NO: 9)
SspDnaB(C-S0)
SPEIEKLSQSDIYWDSIVSITETGVEEVFDLTVPGPHNFVANDIIVHN (SEQ ID NO: 10)
NpuDnaE(N)-LZA
CLSYETEILTVEYGLLPIGKIVEKRIECTVYSVDNNGNIYTQPVAQWHDRGEQEVFEYCLEDGSLIRATKDHKF
MTVDGQMLPIDEIFERELDLMRVDNLPNGGGGSGSAQLEKELQALEKKLAQLEWENQALEKELAQ (SEQ ID
NO: 11)
LZB-NpuDnaGEP(C)
AQLKKKLQANKKELAQLKWKLQALKKKLAQGGGGSGSMIKIATRKYLGKQNVYDIGVGEPHNFALKNGFIA
SN (SEQ ID NO: 12)
NpuDnaGFP(C)
IKIATRKYLGKQNVYDIGVGEPHNFALKNGFIASN (SEQ ID NO: 13)
LZA
AQLEKELQALEKKLAQLEWENQALEKELAQ (SEQ ID NO: 14)
LZB
AQLKKKLQANKKELAQLKWKLQALKKKLAQ (SEQ ID NO: 15)
SspDnaE(N)
CLSFGTEILTVEYGPLPIGKIVSEEINCSVYSVDPEGRVYTQAIAQWHDRGEQEVLEYELEDGSVIRATSDHRFL
TTDYQLLAIEEIFARQLDLLTLENIKQTEEALDNHRLPFPLLDAGTIK (SEQ ID NO: 16)
SspDnaE(C)
VKVIGRRSLGVQRIFDIGLPQDHNFLLANGAIAAN (SEQ ID NO: 17)
SspDnaB(N)
CISGDSLISLA (SEQ ID NO: 18)
SspDnaB(C)
STGKRVSIKDLLDEKDFEIWAINEQTMKLESAKVSRVFCTGKKLVYILKTRLGRTIKATANHRFLTIDGWKRLD
ELSLKEHIALPRKLESSSLQLSPEIEKLSQSDIYWDSIVSITETGVEEVFDLTVPGPHNFVANDIIVHN (SEQ ID
NO: 19)
Plasmid 3: pLX-Hygro(1-89)-NpuDnaE(N)-IRES-TagBFP2
Protein = Hygro(1-89)-NpuDnaE(N)
Vector sequence (SEQ ID NO: 20)
Amino acid sequence (SEQ ID NO: 21)
Plasmid 4: pLX-NpuDnaE(C)-Hygro(90-341)-IRES-mCherry
Protein = NpuDnaE(C)-Hygro(90-341)
Vector sequence (SEQ ID NO: 22)
Amino acid sequence (SEQ ID NO: 23)
Plasmid 5: pLX-Hygro(1-200)-SspDnaB(N)-IRES-TagBFP2
Protein = Hygro(1-200)-SspDnaB(N)
Vector sequence (SEQ ID NO: 24)
Amino acid sequence (SEQ ID NO: 25)
Plasmid 6: pLX-SspDnaB(C)-Hygro(201-341)-IRES-mCherry
Protein = SspDnaB(C)-Hygro(201-341)
Vector sequence (SEQ ID NO: 26)
Amino acid sequence (SEQ ID NO: 27)
Plasmid 7: pLX-Hygro(1-52)-NpuDnaE(N)-IRES-TagBFP2
Protein = Hygro(1-52)-NpuDnaE(N)
Vector sequence (SEQ ID NO: 28)
Amino acid sequence (SEQ ID NO: 29)
Plasmid 8: pLX-NpuDnaE(C)-Hygro(53-341)-IRES-mCheny
Protein = NpuDnaE(C)-Hygro(53-341)
Vector sequence (SEQ ID NO: 30)
Amino acid sequence (SEQ ID NO: 31)
Plasmid 9: pLX-Hygro(1-240)-NpuDnaE(N)-IRES-TagBFP2
Protein = Hygro(1-240)-NpuDnaE(N)
Vector sequence (SEQ ID NO: 32)
Amino acid sequence (SEQ ID NO: 33)
Plasmid 10: pLX-NpuDnaE(C)-Hygro(241-341)-IRES-mCheny
Protein = NpuDnaE(C)-Hygro (241 -341)
Vector sequence (SEQ ID NO: 34)
Amino acid sequence (SEQ ID NO: 35)
Plasmid 11: pLX-Hygro(1-292)-NpuDnaE(N)-IRES-TagBFP2
Protein = Hygro(1-292)-NpuDnaE(N)
Vector sequence (SEQ ID NO: 36)
Amino acid sequence (SEQ ID NO: 37)
Plasmid 12: pLX-NpuDnaE(C)-Hygro(293-341)-IRES-mCheny
Protein = NpuDnaE(C)-Hygro(293-341)
Vector sequence (SEQ ID NO: 38)
Amino acid sequence (SEQ ID NO: 39)
Plasmid 13: pLX-Blast(1-102)-NpuDnaE(N)-IRES-TagBFP2
Protein = Blast(1-102)-NpuDnaE(N)
Vector sequence (SEQ ID NO: 40)
Amino acid sequence (SEQ ID NO: 41)
Plasmid 14: pLX-NpuDnaE(C)-Blast(103-140)-IRES-mCheny
Protein = NpuDnaE(C)-Blast(103-140)
Vector sequence (SEQ ID NO: 42)
Amino acid sequence (SEQ ID NO: 43)
Plasmid 17: pLX-Puro(1-119)-NpuDnaE(N)-IRES-TagBFP2
Protein = Puro(1-119)-NpuDnaE(N)
Vector sequence (SEQ ID NO: 44)
Amino acid sequence (SEQ ID NO: 45)
Plasmid 18: pLX-NpuDnaE(C)-Puro(insCys; 120-199)-IRES-mCheny
Protein = NpuDnaE(C)-Puro(insCys; 120-199)
Vector sequence (SEQ ID NO: 46)
Amino acid sequence (SEQ ID NO: 47)
Plasmid 19: pLX-Puro(1-100)-SspDnaB(N-S0)-IRES-TagBFP2
Protein = Puro(1-100)-SspDnaB(N-S0)
Vector sequence (SEQ ID NO: 48)
Amino acid sequence (SEQ ID NO: 49)
Plasmid 20: pLX-SspDnaB(C-S0)-Puro(101-199)-IRES-mCherry
Protein = SspDnaB(C-S0)-Puro(101-199)
Vector sequence (SEQ ID NO: 50)
Amino acid sequence (SEQ ID NO: 51)
Plasmid 21: pLX-Neo(1-133)-NpuDnaE(N)-IRES-TagBFP2
Protein = Neo(1-133)-NpuDnaE(N)
Vector sequence (SEQ ID NO: 52)
Amino acid sequence (SEQ ID NO: 53)
Plasmid 22: pLX-NpuDnaE(C)-Neo(134-267)-IRES-mCherry
Protein = NpuDnaE(C)-Neo(134-267)
Vector sequence (SEQ ID NO: 54)
Amino acid sequence (SEQ ID NO: 55)
Plasmid 23: pLX-Neo(1-194)-NpuDnaE(N)-IRES-TagBFP2
Protein = Neo(1-194)-NpuDnaE(N)
Vector sequence (SEQ ID NO: 56)
Amino acid sequence (SEQ ID NO: 57)
Plasmid 24: pLX-NpuDnaE(C)-Neo(195-267)-IRES-mCherry
Protein = NpuDnaE(C)-Neo(195-267)
Vector sequence (SEQ ID NO: 58)
Amino acid sequence (SEQ ID NO: 59)
Plasmid 25: pLX-NpuDnaE(C)_Hygro(53-89)-NpuDnaE(N)-IRES-GFP
Protein = NpuDnaE(C)_Hygro(53-89)-NpuDnaE(N)
Vector sequence (SEQ ID NO: 60)
Amino acid sequence (SEQ ID NO: 61)
Plasmid 26: pLX-NpuDnaE(C)_Hygro(53-239)-NpuDnaE(N)-IRES-GFP
Protein = NpuDnaE(C)_Hygro(53-239)-NpuDnaE(N)
Vector sequence (SEQ ID NO: 62)
Amino acid sequence (SEQ ID NO: 63)
Plasmid 27: pCR8-BsaI→ccdbCam←BsaI-NpuDnaE(N)-MD1-68-15 (SEQ ID NO: 64)
Plasmid 28: pCR8-NpuDnaE(C)_BsaI→ccdbCam←BsaI-MD1-68-18 (SEQ ID NO: 65)
Plasmid 29: pCR8-BsaI→ccdbCam←BsaI-SspDnaE(N)-MD1-68-12 (SEQ ID NO: 66)
Plasmid 30: pCR8-SspDnaE(C)_BsaI→ccdbCam←BsaI-MD1-68-13 (SEQ ID NO: 67)
Plasmid 31: pCR8-BsaI→ccdbCam←BsaI-SspDnaB(N-S0)-25-135-18 (SEQ ID NO: 68)
Plasmid 32: pCR8-SspDnaB(C-S0)_BsaI→ccdbCam←BsaI-25-155-41 (SEQ ID NO: 69)
Plasmid 33: pLX-mScarlet(1-46)-NpuDnaE(N)_LZA-IRES-TagBFP2
Protein = mScarlet(1-46)-NpuDnaE(N)_LZA
Vector sequence (SEQ ID NO: 70)
Amino acid sequence (SEQ ID NO: 71)
Plasmid 34: pLX-LZB_NpuDnaE(C)-mScarlet(insCys; 47-232)-IRES-TagBFP2
Protein = LZB_NpuDnaE(C)-mScarlet(insCys; 47-232)
Vector sequence (SEQ ID NO: 72)
Amino acid sequence (SEQ ID NO: 73)
Plasmid 35: pLX-mScarlet(1-48)-NpuDnaE(N)_LZA-IRES-TagBFP2
Protein = mScarlet(1-48)-NpuDnaE(N)_LZA
Vector sequence (SEQ ID NO: 74)
Amino acid sequence (SEQ ID NO: 75)
Plasmid 36: pLX-LZB_NpuDnaE(C)-mScarlet(insCys; 49-232)-IRES-GFP
Protein = LZB_NpuDnaE(C)-mScarlet(insCys; 49-232)
Vector sequence (SEQ ID NO: 76)
Amino acid sequence (SEQ ID NO: 77)
Plasmid 37: pLX-mScarlet(1-51)-NpuDnaE(N)_LZA -IRES-TagBFP2
Protein = mScarlet(1-51)-NpuDnaE(N)_LZA
Vector sequence (SEQ ID NO: 78)
Amino acid sequence (SEQ ID NO: 79)
Plasmid 38: pLX-LZB_NpuDnaE(C)-mScarlet(insCys; 52-232)-IRES-GFP
Protein = LZB_NpuDnaE(C)-mScarlet(insCys; 52-232)
Vector sequence (SEQ ID NO: 80)
Amino acid sequence (SEQ ID NO: 81)
Plasmid 39: pLX-mScarlet(1-75)-NpuDnaE(N)_LZA-IRES-TagBFP2
Protein = mScarlet(1-75)-NpuDnaE(N)_LZA
Vector sequence (SEQ ID NO: 82)
Amino acid sequence (SEQ ID NO: 83)
Plasmid 40: pLX-LZB_NpuDnaE(C)-mScarlet(insCys; 76-232)-IRES-GFP
Protein = LZB_NpuDnaE(C)-mScarlet(insCys; 76-232)
Vector sequence (SEQ ID NO: 84)
Amino acid sequence (SEQ ID NO: 85)
Plasmid 41: pLX-mScarlet(1-122)-NpuDnaE(N)_LZA-IRES-TagBFP2
Protein = mScarlet(1-122)-NpuDnaE(N)_LZA
Vector sequence (SEQ ID NO: 86)
Amino acid sequence (SEQ ID NO: 87)
Plasmid 42: pLX-LZB_NpuDnaE(C)-mScarlet(insCys; 123-232)-IRES-GFP
Protein = LZB_NpuDnaE(C)-mScarlet(insCys; 123-232)
Vector sequence (SEQ ID NO: 88)
Amino acid sequence (SEQ ID NO: 89)
Plasmid 43: pLX-mScarlet(1-140)-NpuDnaE(N)_LZA-IRES-TagBFP2
Protein = mScarlet(1-140)-NpuDnaE(N)_LZA
Vector sequence (SEQ ID NO: 90)
Amino acid sequence (SEQ ID NO: 91)
Plasmid 44: pLX-LZB_NpuDnaE(C)-mScarlet(insCys; 141-232)-IRES-GFP
Protein = LZB_NpuDnaE(C)-mScarlet(insCys; 141-232)
Vector sequence (SEQ ID NO: 92)
Amino acid sequence (SEQ ID NO: 93)
Plasmid 45: pLX-mScarlet(1-163)-NpuDnaE(N)_LZA-IRES-TagBFP2
Protein = mScarlet(1-163)-NpuDnaE(N)_LZA
Vector sequence (SEQ ID NO: 94)
Amino acid sequence (SEQ ID NO: 95)
Plasmid 46: pLX-LZB_NpuDnaE(C)-mScarlet(insCys; 164-232)-IRES-GFP
Protein = LZB_NpuDnaE(C)-mScarlet(insCys; 164-232)
Vector sequence (SEQ ID NO: 96)
Amino acid sequence (SEQ ID NO: 97)
Plasmid 47: pCR8-TagBFP2
Protein = TagBFP2
Vector sequence (SEQ ID NO: 98)
Amino acid sequence (SEQ ID NO: 99)
Plasmid 48: pCR8-mCherry
Protein = mCherry
Vector sequence (SEQ ID NO: 100)
Amino acid sequence (SEQ ID NO: 101)
Plasmid 49: pLX-DEST-IRES-Hygro(1-89)-NpuDnaE(N)
Protein = Hygro(1-89)-NpuDnaE(N)
Vector sequence (SEQ ID NO: 102)
Amino acid sequence (SEQ ID NO: 103)
Plasmid 50: pLX-DEST-IRES-NpuDnaE(C)-Hygro(90-341)
Protein = NpuDnaE(C)-Hygro(90-341)
Vector sequence (SEQ ID NO: 104)
Amino acid sequence (SEQ ID NO: 105)
Plasmid 51: pLX-[TagBFP2]-IRES-Hygro(1-89)-NpuDnaE(N)
Vector sequence (SEQ ID NO: 106)
Plasmid 52: pLX-[mCherry]-IRES-NpuDnaE(C)-Hygro(90-341)
Vector sequence (SEQ ID NO: 107)
Plasmid 53: pLX-DEST-IRES-Puro(1-119)-NpuDnaE(N)
Protein = Puro(1-119)-NpuDnaE(N)
Vector sequence (SEQ ID NO: 108)
Amino acid sequence (SEQ ID NO: 109)
Plasmid 54: pLX-DEST-IRES-NpuDnaE(C)-Puro(120-199)
Protein = NpuDnaE(C)-Puro(120-199)
Vector sequence (SEQ ID NO: 110)
Amino acid sequence (SEQ ID NO: 111)
Plasmid 55: pLX-[TagBFP2]-IRES-Puro(1-119)-NpuDnaE(N)
Vector sequence (SEQ ID NO: 112)
Plasmid 56: pLX-[mCherry]-IRES-NpuDnaE(C)-Puro(120-199)
Vector sequence (SEQ ID NO: 113)
Plasmid 57: pLX-DEST-IRES-Neo(1-194)-NpuDnaE(N)
Protein = Neo(1-194)-NpuDnaE(N)
Vector sequence (SEQ ID NO: 114)
Amino acid sequence (SEQ ID NO: 115)
Plasmid 58: pLX-DEST-IRES-NpuDnaE(C)-Neo(195-267)
Protein = NpuDnaE(C)-Neo(195-267)
Vector sequence (SEQ ID NO: 116 )
Amino acid sequence (SEQ ID NO: 117)
Plasmid 59: pLX-[TagBFP2]-IRES-Neo(1-194)-NpuDnaE(N)
Vector sequence (SEQ ID NO: 118)
Plasmid 60: pLX-[mCherry]-IRES-NpuDnaE(C)-Neo(195-267)
Vector sequence (SEQ ID NO: 119)
Plasmid 61: pLX-mScarlet(1-51)-NpuDnaE(N)-LZA-IRES-TagBFP2
Protein = mScarlet(1-51)-NpuDnaE(N)-LZA
Vector sequence (SEQ ID NO: 120)
Amino acid sequence (SEQ ID NO: 121)
Plasmid 62: pLX-LZB-NpuDnaE(C)-mScarlet(\hat{ }C,52-163)-NpuDnaE(N)_LZA-IRES-EGFP
Protein = LZB-NpuDnaE(C)-mScarlet(C; 52-163)-NpuDnaE(N)_LZA
Vector sequence (SEQ ID NO: 122)
Amino acid sequence (SEQ ID NO: 123)
Plasmid 63: pLX-LZB-NpuDnaE(C)-mScarlet(\hat{ }C; 164-232)-IRES-EGFP
Protein = LZB-NpuDnaE(C)-mScarlet(\hat{ }C; 164-232)
Vector sequence (SEQ ID NO: 124)
Amino acid sequence (SEQ ID NO: 125)
Plasmid 64: pLX-Hygro(1 -69)-NpuDnaE(N)-IRES-TagBFP2
Protein = Hygro(1-69)-NpuDnaE(N)
Vector sequence (SEQ ID NO: 126)
Amino acid sequence (SEQ ID NO: 127)
Plasmid 65: pLX-NpuDnaE(C)-Hygro(\hat{ }C; 70-341)-IRES-mCherry
Protein = NpuDnaE(C)-Hygro(\hat{ }C; 70-341)
Vector sequence (SEQ ID NO: 128)
Amino acid sequence (SEQ ID NO: 129)
Plasmid 66: pLX-Hygro(1-131)-NpuDnaE(N)-IRES-TagBFP2
Protein = Hygro(1-131)-NpuDnaE(N)
Vector sequence (SEQ ID NO: 130)
Amino acid sequence (SEQ ID NO: 131)
Plasmid 67: pLX-NpuDnaE(C)-Hygro(\hat{ }C; 132-341)-IRES-mCherry
Protein = NpuDnaE(C)-Hygro(\hat{ }C; 132-341)
Vector sequence (SEQ ID NO: 132)
Amino acid sequence (SEQ ID NO: 133)
Plasmid 68: pLX-Hygro(1-171)-NpuDnaE(N)-IRES-TagBFP2
Protein = Hygro(1-171)-NpuDnaE(N)
Vector sequence (SEQ ID NO: 134)
Amino acid sequence (SEQ ID NO: 135)
Plasmid 69: pLX-NpuDnaE(C)-Hygro(\hat{ }C; 172-341)-IRES-mCherry
Protein = NpuDnaE(C)-Hygro(\hat{ }C; 172-341)
Vector sequence (SEQ ID NO: 136)
Amino acid sequence (SEQ ID NO: 137)
Plasmid 70: pLX-Hygro(1-218)-NpuDnaE(N)-IRES-TagBFP2
Protein = Hygro(1-218)-NpuDnaE(N)
Vector sequence (SEQ ID NO: 138)
Amino acid sequence (SEQ ID NO: 139)
Plasmid 71: pLX-NpuDnaE(C)-Hygro(\hat{ }C; 219-341)-IRES-mCherry
Protein = NpuDnaE(C)-Hygro(\hat{ }C; 219-341)
Vector sequence (SEQ ID NO: 140)
Amino acid sequence (SEQ ID NO: 141)
Plasmid 72: pLX-Hygro(1-259)-NpuDnaE(N)-IRES-TagBFP2
Protein = Hygro(1-259)-NpuDnaE(N)
Vector sequence (SEQ ID NO: 142)
Amino acid sequence (SEQ ID NO: 143)
Plasmid 73: pLX-NpuDnaE(C)-Hygro(\hat{ }C; 260-341)-IRES-mCherry
Protein = NpuDnaE(C)-Hygro(\hat{ }C; 260-341)
Vector sequence (SEQ ID NO: 144)
Amino acid sequence (SEQ ID NO: 145)
Plasmid 74: pLX-Hygro(1-277)-NpuDnaE(N)-IRES-TagBFP2
Protein = Hygro(1-277)-NpuDnaE(N)
Vector sequence (SEQ ID NO: 146)
Amino acid sequence (SEQ ID NO: 147)
Plasmid 75: pLX-NpuDnaE(C)-Hygro(\hat{ }C; 278-341)-IRES-mCherry
Protein = NpuDnaE(C)-Hygro(\hat{ }C; 278-341)
Vector sequence (SEQ ID NO: 148)
Amino acid sequence (SEQ ID NO: 149)
Plasmid 76: pLX-Puro(1-32)-NpuDnaE(N)-IRES-TagBFP2
Protein = Puro(1-32)-NpuDnaE(N)
Vector sequence (SEQ ID NO: 150)
Amino acid sequence (SEQ ID NO: 151)
Plasmid 77: pLX-NpuDnaE(C)-Puro(\hat{ }C; 33-199)-IRES-mCherry
Protein = NpuDnaE(C)-Puro(\hat{ }C; 33-199)
Vector sequence (SEQ ID NO: 152)
Amino acid sequence (SEQ ID NO: 153)
Plasmid 78: pLX-Puro(1-84)-NpuDnaE(N)-IRES-TagBFP2
Protein = Puro(1-84)-NpuDnaE(N)
Vector sequence (SEQ ID NO: 154)
Amino acid sequence (SEQ ID NO: 155)
Plasmid 79: pLX-NpuDnaE(C)-Puro(\hat{ }C; 85-199)-IRES-mCherry
Protein = NpuDnaE(C)-Puro(\hat{ }C; 85-199)
Vector sequence (SEQ ID NO: 156)
Amino acid sequence (SEQ ID NO: 157)
Plasmid 80: pLX-Puro(1-137)-NpuDnaE(N)-IRES-TagBFP2
Protein = Puro(1-137)-NpuDnaE(N)
File = pLX-[PuroKC3(N)-NpuDnaE(N)-25-131-29″]-IRES-TagBFP2-25-133-6
Vector sequence (SEQ ID NO: 158)
Amino acid sequence (SEQ ID NO: 159)
Plasmid 81: pLX-NpuDnaE(C)-Puro(\hat{ }C; 138-199)-IRES-mCherry
Protein = NpuDnaE(C)-Puro(\hat{ }C; 138-199)
Vector Sequence (SEQ ID NO: 160)
Amino acid sequence (SEQ ID NO: 161)
Plasmid 82: pLX-Puro(1-158)-NpuDnaE(N)-IRES-TagBFP2
Protein = Puro(1-158)-NpuDnaE(N)
Vector Sequence (SEQ ID NO: 162)
Amino acid sequence (SEQ ID NO: 163)
Plasmid 83: pLX-NpuDnaE(C)-Puro(\hat{ }C; 159-199)-IRES-mCherry
Protein = NpuDnaE(C)-Puro(\hat{ }C; 159-199)
Vector Sequence (SEQ ID NO: 164)
Amino acid sequence (SEQ ID NO: 165)
Plasmid 84: pLX-Puro(1-180)-NpuDnaE(N)-IRES-TagBFP2
Protein = Puro(1-180)-NpuDnaE(N)
Vector Sequence (SEQ ID NO: 166)
Amino acid sequence (SEQ ID NO: 167)
Plasmid 85: pLX-NpuDnaE(C)-Puro(\hat{ }C; 181-199)-IRES-mCherry
Protein = NpuDnaE(C)-Puro(\hat{ }C; 181-199)
Vector Sequence (SEQ ID NO: 168)
Amino acid sequence (SEQ ID NO: 169)
Plasmid 86: pLX-Blast(1-58)-NpuDnaE(N)-IRES-TagBFP2
Protein = Blast(1-58)-NpuDnaE(N)
Vector Sequence (SEQ ID NO: 170)
Amino acid sequence (SEQ ID NO: 171)
Plasmid 87: pLX-NpuDnaE(C)-Blast(59-140)-IRES-mCherry
Protein = NpuDnaE(C)-Blast(59-140)
Vector Sequence (SEQ ID NO: 172)
Amino acid sequence (SEQ ID NO: 173)
Plasmid 88: pLX-NpuDnaE((C)-HygroBA-SspDnaB(N-S0)-IRES-EGFP
Protein = NpuDnaE(C)-Hygro(53-200)-SsrsDnaB(N-S0)
Vector Sequence (SEQ ID NO: 174)
Amino acid sequence (SEQ ID NO: 175)
Plasmid 89: pLX-SspDnaB(C-S0)-Hygro(201-341)-IRES-mCherry
Protein = SspDnaB(C-S0)-Hygro(201-341)
Vector Sequence (SEQ ID NO: 176)
Amino acid sequence (SEQ ID NO: 177)
Plasmid 90: pLX-NpuDnaE(C)-Hygro(90-200)-SspDnaB(N-S0)-IRES-EGFP
Protein = NpuDnaE(C)-Hygro(90-200)-SspDnaB(N-S0)
Vector Sequence (SEQ ID NO: 178)
Amino acid sequence (SEQ ID NO: 179)
Plasmid 91: pLX-Hygro(1-200)-SspDnaB(N-S0)-IRES-TagBFP2
Protein = Hygro(1-200)-SspDnaB(N-S0)
Vector sequence (SEQ ID NO: 180)
Amino acid sequence (SEQ ID NO: 181)
Plasmid 92: pLX-SspDnaB(C-S0)-Hygro(201-240)-NpuDnaE(N)-IRES-EGFP
Protein = SspDnaB(C-S0)-Hygro(201-240)-NpuDnaE(N)
Vector sequence (SEQ ID NO: 182)
Amino acid sequence (SEQ ID NO: 183)
Plasmid 93: pLX-SspDnaB(C-S0)-Hygro(201-292)-NpuDnaE(N)-IRES-EGFP
Protein = SspDnaB(C-S0)-Hygro(201-292)-NpuDnaE(N)
Vector sequence (SEQ ID NO: 184)
Amino acid sequence (SEQ ID NO: 185)
Plasmid 94: pLX-DEST-IRES-TagBFP2 (SEQ ID NO: 186)
Plasmid 95: pLX-DEST-IRES-EGFP (SEQ ID NO: 187)
Plasmid 96: pLX-DEST-IRES-mCherry (SEQ ID NO: 188)
Plasmid 97: pLX-Hygro-IRES-TagBFP2
Vector sequence (SEQ ID NO: 189)
Plasmid 98: pLX-Hygro-IRES-mCherry
Vector sequence (SEQ ID NO: 190)
Plasmid 99: pLX-Puro-IRES-TagBFP2
Vector sequence (SEQ ID NO: 191)
Plasmid 100: pLX-Puro-IRES-mCherry
Vector sequence (SEQ ID NO: 192)
Plasmid 101: pLX-Hygro-IRES-EGFP
Vector sequence (SEQ ID NO: 193)
Plasmid 102: pLX-NLS_GFP-IRES-Hygro
Vector sequence (SEQ ID NO: 194)
Plasmid 103: pLX-LifeAct_mCherry-IRES-Hygro
Vector sequence (SEQ ID NO: 195)
Plasmid 104: pLX-NLS_GFP-IRES-Hygro(1-89)-NpuDnaE(N)
Vector sequence (SEQ ID NO: 196)
Plasmid 105: pLX-LifeAct_mScarlet-IRES-NpuDnaE(C)-Hygro(90-341)
Vector sequence (SEQ ID NO: 197)
Plasmid 106: pX330-AAVS1
sgRNA spacer sequence: gACCCCACAGTGGGGCCACTA (First g does not match genome)(SEQ ID NO: 198)
Vector sequence (SEQ ID NO: 199)
Plasmid 107: pAAVS1-Nst-EF1aHygro2ArtTA3(-)_TetO-Blast-P2A-EGFP
Vector sequence (SEQ ID NO: 200)
Plasmid 108: pAAVS1-Nst-EF1aHygro2ArtTA3(-)_TetO-Blast-P2A-mScarlet
Vector sequence (SEQ ID NO: 201)
Plasmid 109: pAAVS1-Nst-EF1aHygro2ArtTA3(-)_TetO-Blast(1-102)_NpuDnaE(N)-P2A-EGFP
Vector sequence (SEQ ID NO: 202)
Plasmid 110: pAAVS1-Nst-EF1aHygro2ArtTA3(-)_TetO-NpuDnaE(C)_Blast(103-140)-P2A-mScarlet
Vector sequence (SEQ ID NO: 203)
Plasmid 111: pAAVS1-Nst-EF1aBlast2ArtTA3(-)_TetO-Hygro-P2A-NTR-E2A-EGFP
Vector sequence (SEQ ID NO: 204)
Plasmid 112: pAAVS1-Nst-EF1aBlast2ArtTA3(-)_TetO-Hygro-P2A-NTR-E2A-mCherry
Vector sequence (SEQ ID NO: 205)
Plasmid 113: pAAVS1-Nst-EF1aBlast2ArtTA3(-)_TetO-Hygro(1-89)-NpuDnaE(N)-P2A-NTR-E2A-EGFP
Vector sequence (SEQ ID NO: 206)
Plasmid 114: pAAVS1-Nst-EF1aBlast2ArtTA3(-)_TetO-NpuDnaE(C)-Hygro(90-341)-P2A-NTR-E2A-mCherry
Vector sequence (SEQ ID NO: 207)
Plasmid 115: pLX-Hygro(1-89)_NpuDnaE(N)_LZA-IRES-TagBFP2
Protein = Hygro(1-89)-NpuDnaE(N)-LZA
Vector sequence (SEQ ID NO: 208)
Amino acid sequence (SEQ ID NO: 209)
Plasmid 116: pLX-LZB_NpuDnaGEP(C)_Hygro(90-200)_SspDnaB(N-S0)-IRES-GFP
Protein = LZB-NpuDnaGEP(C)-Hygro(90-200)-SspDnaB(N-S0)
Vector sequence (SEQ ID NO: 210)
Amino acid sequence (SEQ ID NO: 211)
Plasmid 117: pLX-SspDnaB(C-S0)_Hygro(201-240)_NpuDnaE(N)_LZA-IRES-GFP
Protein = SspDnaB(C-S0)-Hygro(201-240)-NpuDnaE(N)-LZA
Vector sequence (SEQ ID NO: 212)
Amino acid sequence (SEQ ID NO: 213)
Plasmid 118: pLX-LZB_NpuDnaGEP(C)_Hygro(241-341)-IRES-mCherry
Protein = LZB-NpuDnaGEP(C)-Hygro(241-341)
Vector sequence (SEQ ID NO: 214)
Amino acid sequence (SEQ ID NO: 215)
AC1947GB (SEQ ID NO: 216)
AC1949GB (SEQ ID NO: 217)
pCR8-ccdbCam (SEQ ID NO: 218)

### REFERENCES

1. Shearer, R. F. & Saunders, D. N. Experimental design for stable genetic manipulation in mammalian cell lines: lentivirus and alternatives. Genes to cells: devoted to molecular & cellular mechanisms 20, 1-10 (2015).
2. Abuin, A. & Bradley, A. Recycling selectable markers in mouse embryonic stem cells. Molecular and cellular biology 16, 1851-1856 (1996).
3. Shah, N. H. & Muir, T. W. Inteins: Nature's Gift to Protein Chemists. Chemical science 5, 446-461 (2014).
4. Zettler, J., Schutz, V. & Mootz, H. D. The naturally split Npu DnaE intein exhibits an extraordinarily high rate in the protein trans-splicing reaction. FEBS letters 583, 909-914 (2009).
5. Iwai, H., Züger, S., Jin, J. & Tam, P.-H. Highly efficient protein trans-splicing by a naturally split DnaE intein from Nostoc punctiforme. FEBS letters 580, 1853-1858 (2006).
6. Sun, W., Yang, J. & Liu, X. Q. Synthetic two-piece and three-piece split inteins for protein trans-splicing. The Journal of biological chemistry 279, 35281-35286 (2004).
7. Cheriyan, M., Pedamallu, C. S., Tori, K. & Perler, F. Faster protein splicing with the Nostoc punctiforme DnaE intein using non-native extein residues. The Journal of biological chemistry 288, 6202-6211 (2013).
8. Chee, J. & Chin, C. Gateway cloning technology: Advantages and drawbacks. Cloning Transgenes 4, 138 (2015).
9. Bindels, D. S. et al. mScarlet: a bright monomeric red fluorescent protein for cellular imaging. nature methods 14, 53 (2017).
10. Stevens, A. J. et al. A promiscuous split intein with expanded protein engineering applications. Proceedings of the National Academy of Sciences 114, 8538-8543 (2017).
11. Ghosh, I., Hamilton, A. D. & Regan, L. Antiparallel leucine zipper-directed protein reassembly: application to the green fluorescent protein. Journal of the American Chemical Society 122, 5658-5659 (2000).
12. Wang, H., La Russa, M. & Qi, L. S. CRISPR/Cas9 in genome editing and beyond. Annual review of biochemistry 85, 227-264 (2016).
13. Peng, R., Lin, G. & Li, J. Potential pitfalls of CRISPR/Cas9-mediated genome editing. The FEBS journal 283, 1218-1231 (2016).
14. Oceguera-Yanez, F. et al. Engineering the AAVS1 locus for consistent and scalable transgene expression in human iPSCs and their differentiated derivatives. Methods 101, 43-55 (2016).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising delivering to eukaryotic cells
(a) a first vector comprising (i) a nucleotide sequence encoding an N-terminal fragment of a selectable marker protein, which is upstream from a nucleotide sequence encoding an N-terminal fragment of an intein and (ii) a nucleotide sequence encoding a first molecule of interest; and
(b) a second vector comprising (i) a nucleotide sequence encoding a C-terminal fragment of the intein, which is upstream from a C-terminal fragment of the selectable marker protein and (ii) a nucleotide sequence encoding a second molecule of interest,
wherein the N-terminal fragment and the C-terminal fragment of the intein catalyze joining of the N-terminal fragment and the C-terminal fragment of the selectable marker protein to produce a full-length selectable marker protein.

2. The method of claim 1 further comprising maintaining the eukaryotic cells under conditions that permit introduction of the first and second vectors into the eukaryotic cells to produce transgenic eukaryotic cells.

3. The method of claim 2 further comprising selecting the transgenic eukaryotic cells that comprise the full-length selectable marker protein.

4. The method of any one of claims 1-3, wherein the eukaryotic cells are mammalian cells.

5. The method of any one of claims 1-4, wherein the selectable marker protein is an antibiotic resistance protein.

6. The method of claim 5, wherein the antibiotic resistance protein confers resistance to hygromycin, G418, puromycin, phleomycin D1 or blasticidin.

7. The method of claim 5 or 6, wherein the antibiotic resistance protein is encoded by a hygB gene, a bsr gene, a pac gene, or a neo gene.

8. The method of any one of claims 1-4, wherein the selectable marker protein is a fluorescent protein.

9. The method of claim 8, wherein the fluorescent protein is selected from TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mScarlet, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.

10. The method of any one of claims 1-9, wherein the intein is a split intein.

11. The method of claim 10, wherein the split intein is a natural split, optionally wherein the natural split intein is selected from DnaE inteins, and optionally wherein the DnaE inteins are selected from Synechocystis sp. DnaE (SspDnaE) inteins and Nostoc punctiforme (NpuDnaE) inteins.

12. The method of claim 10, wherein the split intein is an engineered split intein, optionally wherein the engineered split intein is engineered from DnaB inteins or GyrB inteins, and optionally wherein the engineered split intein is a SspDnaB S1 intein or a SspGyrB S11 intein.

13. The method of any one of claims 1-12, wherein the first and/or second molecule is a protein or a non-coding ribonucleic acid (RNA), optionally wherein the non-coding RNA is a microRNA (miRNA), antisense RNA, short-interfering RNA (siRNA) or short-hairpin RNA (shRNA).

14. The method of any one of claims 1-13, wherein the first and/or second vector is a plasmid vector or a viral vector.

15. A eukaryotic cell, comprising
(a) a first vector comprising (i) a nucleotide sequence encoding an N-terminal fragment of a selectable marker protein, which is upstream from a nucleotide sequence encoding an N-terminal fragment of an intein and (ii) a nucleotide sequence encoding a first molecule of interest; and
(b) a second vector comprising (i) a nucleotide sequence encoding a C-terminal fragment of the intein, which is upstream from a C-terminal fragment of the selectable marker protein and (ii) a nucleotide sequence encoding a second molecule of interest,
wherein the N-terminal fragment and the C-terminal fragment of the intein catalyze joining of the N-terminal fragment and the C-terminal fragment of the selectable marker protein to produce a full-length antibiotic resistance protein.

16. A kit, comprising
(a) a first vector comprising a nucleotide sequence encoding an N-terminal fragment of a selectable marker protein, which is upstream from a nucleotide sequence encoding an N-terminal fragment of an intein; and
(b) a second vector comprising a nucleotide sequence encoding a C-terminal fragment of the intein, which is upstream from a C-terminal fragment of the selectable marker protein,
wherein the N-terminal fragment and the C-terminal fragment of the intein catalyze joining of the N-terminal fragment and the C-terminal fragment of the selectable marker protein to produce a full-length selectable marker protein.

17. A method comprising delivering to eukaryotic cells
(a) a first vector comprising (i) a nucleotide sequence encoding an N-terminal fragment of a selectable marker protein, which is upstream from a nucleotide sequence encoding an N-terminal fragment of a first intein and (ii) a nucleotide sequence encoding a first molecule of interest,
(b) a second vector comprising (i) a nucleotide sequence encoding a C-terminal fragment of the first intein, which is upstream from a nucleotide sequence encoding a central fragment of the selectable marker protein, which is upstream from a nucleotide sequence encoding an N-terminal fragment of a second intein and (ii) a nucleotide sequence encoding a second molecule of interest, and
(c) a third vector comprising (i) a nucleotide sequence encoding a C-terminal fragment of the second intein, which is upstream from a nucleotide sequence encoding a C-terminal fragment of the selectable marker protein and (ii) a nucleotide sequence encoding a third molecule of interest,
wherein the N-terminal fragment and the C-terminal fragment of the first intein catalyze joining of N-terminal fragment of the selectable marker protein to the central fragment of the selectable marker protein, and the N-terminal fragment and the C-terminal fragment of the second intein catalyze joining of central fragment of the selectable marker protein to the C-terminal fragment of the selectable marker protein, to produce a full-length selectable marker protein.

18. The method of claim 19 further comprising maintaining the eukaryotic cells under conditions that permit introduction of the first, second, and third vectors into the eukaryotic cells to produce transgenic eukaryotic cells.

19. The method of claim 18 further comprising selecting the transgenic eukaryotic cells that comprise the full-length selectable marker protein.

20. A eukaryotic cell comprising:
(a) a first vector comprising (i) a nucleotide sequence encoding an N-terminal fragment of a selectable marker protein, which is upstream from a nucleotide sequence encoding an N-terminal fragment of a first intein and (ii) a nucleotide sequence encoding a first molecule of interest,
(b) a second vector comprising (i) a nucleotide sequence encoding a C-terminal fragment of the first intein, which is upstream from a nucleotide sequence encoding a central fragment of the selectable marker protein, which is upstream from a nucleotide sequence encoding an N-terminal fragment of a second intein and (ii) a nucleotide sequence encoding a second molecule of interest, and
(c) a third vector comprising (i) a nucleotide sequence encoding a C-terminal fragment of the second intein, which is upstream from a nucleotide sequence encoding a C-terminal fragment of the selectable marker protein and (ii) a nucleotide sequence encoding a third molecule of interest,
wherein the N-terminal fragment and the C-terminal fragment of the first intein catalyze joining of N-terminal fragment of the selectable marker protein to the central fragment of the selectable marker protein, and the N-terminal fragment and the C-terminal fragment of the second intein catalyze joining of central fragment of the selectable marker protein to the C-terminal fragment of the selectable marker protein, to produce a full-length selectable marker protein.

21. A kit comprising:
(a) a first vector comprising a nucleotide sequence encoding an N-terminal fragment of a selectable marker protein, which is upstream from a nucleotide sequence encoding an N-terminal fragment of a first intein,
(b) a second vector comprising a nucleotide sequence encoding a C-terminal fragment of the first intein, which is upstream from a nucleotide sequence encoding a central fragment of the selectable marker protein, which is upstream from a nucleotide sequence encoding an N-terminal fragment of a second intein, and
(c) a third vector comprising a nucleotide sequence encoding a C-terminal fragment of the second intein, which is upstream from a nucleotide sequence encoding a C-terminal fragment of the selectable marker protein,
wherein the N-terminal fragment and the C-terminal fragment of the first intein catalyze joining of N-terminal fragment of the selectable marker protein to the central fragment of the selectable marker protein, and the N-terminal fragment and the C-terminal fragment of the second intein catalyze joining of central fragment of the selectable marker protein to the C-terminal fragment of the selectable marker protein, to produce a full-length selectable marker protein.
